(19) United States
(12) Patent Application Publication
(10) Pub. No.: US 2025/0136691 A1
(43) Pub. Date: May 1, 2025

(54) GENETICALLY MODIFIED ORGANISM FOR RECOMBINANT PROTEIN PRODUCTION

(71) Applicant: PLANTIBODIES, Paris (FR)

(72) Inventors: Pierre BAUËR, Paris (FR); Bilal MAZHAR, Paris (FR); Won KIM, Paris (FR)

(73) Assignee: PLANTIBODIES, Paris (FR)

(21) Appl. No.: 18/691,368

(22) PCT Filed: Sep. 12, 2022
(86) PCT No.: PCT/EP2022/075293

§ 371 Date: Mar. 12, 2024

(30) Foreign Application Priority Data
Sep. 13, 2021 (EP) 21306255.7

(51) Int. Cl. C07K 16/28; C12N 9/12; C12N 15/82

(52) U.S. Cl. CPC C07K 16/2818; C12N 9/1205; C12N 15/8205; C12N 15/8258; C12N 15/8281; C12Y 207/01095; C07K 2317/24

(57) ABSTRACT

The present invention relates to a method for producing a genetically modified plant, plant cell, protoplast, or plant tissue expressing a recombinant protein of interest, comprising a at least a step of introducing a plant nucleic acid construct that provides for stable expression of the protein of interest into the plant, plant cell, protoplast, or plant tissue; wherein the plant is selected from the Spinacia, Lactuca, and Brassica genus, preferably the plant is an edible plant from the Brassica genus, and the nucleic acid construct is a single synthetic construct comprising a regulatory sequence active in a plant for the expression of an operably linked DNA, an operably linked protein-coding DNA molecule, and a 3′ untranslated region. The present invention encompasses also a nucleic acid, a bacterial strain and a plant obtained according to the method above, as well as a plant produced protein obtained from a genetically modified plant of the present invention.

### FIELD OF THE INVENTION

The present application relates to genetically modified organisms for the production of recombinant protein and methods for obtaining thereof.

### BACKGROUND OF THE INVENTION

To treat chronic diseases, the biopharma industry strives to develop new and innovative classes of drugs. Historically they have developed chemotherapy drugs to treat and retard cancerous growth. However, in recent decades new strategies such as immunotherapies using monoclonal antibodies have been developed (Kunert & Reinhart, 2016, #). These antibodies are typically manufactured/produced using mammalian cell lines (HeLa, CHO, etc) (Kelley, 2009, #).

This classical manufacturing method has been successful in developing various antibodies. Indeed, the majority of approved recombinant biopharmaceuticals, in particular some of the mostly used monoclonal antibodies (mAbs) such as adalimumab (an anti-TNFα mAb) or Nivolumab (an anti-PD-1 mAb), are produced in mammalian cell lines (Rajan A, Kim C, Heery C R, Guha U, Gulley J L. Nivolumab, anti-programmed death-1 (PD-1) monoclonal antibody immunotherapy: Role in advanced cancers. Hum Vaccin Immunother. 2016; 12 (9): 2219-2231.). However, this approach has limitations, due to complex infrastructural needs and operation costs. In addition, to cost, there are also technical and safety constraints (Kelley B. Industrialization of mAb production technology: the bioprocessing industry at a crossroads. MAbs. 2009; 1 (5): 443-452.). For example, bioproduction facilities can easily get contaminated by fungi or Mycoplasma, resulting in the discarding of the production batch (Nikfarjam L, Farzaneh P. Prevention and detection of Mycoplasma contamination in cell culture. Cell J. 2012; 13 (4): 203-212.) (Nation Research Custom Media & Sartorius, n.d.). The production of large scale, quality, affordable, and safe recombinant proteins, notably antibodies are however priorities for the biopharma industry which still remains to be improved.

Plants offer several potential benefits over conventional expression platforms and prove the reliability of the system for the production of highly valuable proteins. Indeed, it has been shown that biologics (i.e., drugs that are made from biological starting material, including those produced using recombinant DNA procedure), including modified human proteins, can be produced in transgenic plants in order to address problems of safety, viral infections, immune reactions, production yield and cost. U.S. Pat. No. 6,391,638 and PCT WO2008/135991 teach bioreactor devices for commercial-scale production of recombinant polypeptides from plant cell culture. U.S. Pat. No. 7,951,557, U.S. patent application Ser. Nos. 10/554,387 and 11/790,991 teach construction and expression of nucleic acid vectors for recombinant expression of human proteins in plant cells. PCT WO2007/010533 teaches the expression of recombinant human polypeptides in plant cells, for enteral administration. Additional background art includes: U.S. Pat. No. 7,915,225 to Finck et al, U.S. patent application Ser. Nos. 13/021,545 and 10/853,479 to Finck et al, U.S. patent application Ser. No. 11/906,600 to Li et al, U.S. patent application Ser. No. 10/115,625 to Warren et al and U.S. patent application Ser. No. 11/784,538 to Gornbotz et al. Therefore, advancements in plant molecular farming approaches in the recent decade have made plants an attractive manufacturing system that can even achieve commercially relevant production levels in a short period, and many plant-produced therapeutic proteins are in pre-clinical and clinical trials and are close to commercialization (Paul, M.; Ma, J. K. Plant-made pharmaceuticals: Leading products and production platforms. Biotechnol. Appl. Biochem. 2011, 58, 58-67).

However, there is still a need in this emerging field for improved reliable methods allowing to obtain higher yields of recombinant protein, in particular in the case of complex multimeric proteins such as antibodies.

Most of the recombinant plant-produced recombinant proteins and in particular recombinant antibodies have been obtained from genetically modified tobacco or from cell lines. Furthermore, to optimize yields, competitors have often favoured transient expression. In the case of plant-produced antibodies, 2 plasmids are typically used and/or progenies of plants expressing light chain and heavy chains respectively need to be crossed to obtain the full antibody (see Edgue G, Twyman R M, Beiss V, Fischer R, Sack M. Antibodies from plants for bionanomaterials. Wiley Interdiscip Rev Nanomedicine Nanobiotechnology. 2017; 9 (6): e1462; Rattanapisit K, Phakham T, Buranapraditkun S, et al. Structural and In Vitro Functional Analyses of Novel Plant-Produced Anti-Human PD1 Antibody. Sci Rep. 2019; 9 (1): 15205; Shanmugaraj B, I. Bulaon C J, Phoolcharoen W. Plant Molecular Farming: A Viable Platform for Recombinant Biopharmaceutical Production. Plants. 2020; 9 (7): 842).

Therefore there remains a need for a faster and reliable method for producing recombinant protein in particular multimeric proteins such as antibodies, in a plant, as well as for genetically modified plants producing multimeric proteins, in particular biosimilar antibodies with high yield and safety.

### SUMMARY OF THE INVENTION

The present invention provides a response to the technical problems previously raised and describes an innovative method for producing recombinant protein, in particular multimeric proteins, in a plant.

Using chemically synthesized nucleic acid construct (i.e., de novo synthesized), the inventor now designed a method for obtaining plant-produced proteins, and in particular plant-produced biosimilar antibodies which is faster, more reliable. Indeed, not only de novo synthesis is less prone to DNA mutation as compared to traditional techniques of the art, but the inventors were able to combine the use of large single nucleic acid construct (>2000 bp) to stably transform a plant. Multimeric proteins are therefore produced according to the present invention from a single nucleic acid construct (or expression vector), where a combination of vectors is classically used. Thus, said multimeric protein (typically an antibody) can be produced from a single genetically modified plant according to the present invention.

Lastly, the plant-produced proteins of the present invention are typically produced in whole plants, notably in edible plants with an important biomass production further allowing high yield of production.

Therefore, the present application relates to a method for producing a genetically modified plant, plant cell, protoplast, or plant tissue expressing a recombinant protein of interest, comprising at least a step of introducing a plant nucleic acid construct that provides for stable expression of the protein of interest into the plant, plant cell, protoplast, or plant tissue;

wherein:
the plant is selected from the Spinacia, Lactuca, and Brassica genus, preferably the plant is an edible plant from the Brassica genus, and
the nucleic acid construct is a single synthetic construct comprising a regulatory sequence active in a plant for the expression of an operably linked DNA, an operably linked protein-coding DNA molecule, and a 3′ untranslated region.

Typically, the method also comprises a further step of obtaining a transgenic plant comprising the DNA construct that stably expresses the protein of interest by regenerating the transgenic plant from the plant, plant cell, protoplast, or plant tissue that received the nucleic acid construct.

Typically, the nucleic acid construct further comprises one or more of the following sequences: a 5′ untranslated sequence, a signal sequence, an enhancer sequence, a cis-acting element, an intron sequence, a transcriptional Terminator Sequence (TTS), and one or more selectable marker coding sequences.

In some embodiments, the protein of interest is a multimeric protein, optionally wherein the protein of interest is an antibody. More particularly according to the present invention, the protein of interest is an antibody, and the protein-coding DNA molecule is a single synthetic molecule comprising a nucleic acid sequence coding for an antibody light chain or the antigen-binding fragment thereof and a nucleic acid sequence coding for an antibody heavy chain or the antigen-binding fragment thereof. In some embodiments, the antibody is an anti-PD-1, notably the antibody is nivolumab.

In some embodiments, the multimeric protein-coding nucleic acid synthetic molecule comprises 2 tag sequences located at the 3′ end of each monomer coding sequence, optionally the antibody-coding DNA synthetic molecule comprises a Tag sequence at the 3′ end of the light chain coding sequence and a Tag sequence at the 3′ end of heavy chain coding sequence, optionally the protein-coding nucleic acid molecule code for a protein sequence having at least 90% identity with sequence of SEQ ID NO:3, optionally the protein-coding nucleic acid molecule has at least 60% (notably 65, 70, 75, 80, 85, 90, 95, 99 or 100%) identity with the nucleic acid sequence of SEQ ID NO:3 and typically it codes for a protein having at least 90% identity with sequence of SEQ ID NO:3

In some embodiments, the nucleic acid construct can be introduced into the plant or plant cells using
(i) a direct DNA uptake method or
(ii) Agrobacterium-mediated plant transformation, typically wherein the nucleic acid construct is inserted between the DNA border repeats of an Agrobacterium-mediated plant transformation binary vector.

In some embodiments, the method further comprises preliminary steps of:
a1) preparing a transformant by introducing a nucleic acid construct into a bacterial strain; and
a2) transforming the plant, plant cell, protoplast, or plant tissue using the transformant.

Typically, according to the present invention, the strain is an Agrobacterium strain, notably an A. tumefaciens strain. In some embodiments, at step a1) the bacterial strain is obtained using a binary vector system, wherein the bacterial strain is co-transfected with a T DRNA binary vector as previously defined and a vic helper plasmid, or wherein a T DNA disarmed A. tumefaciens strain is transfected with a T/DNA binary vector as previously defined.

The present invention also encompasses a method for obtaining a plant produced protein comprising:
producing a genetically modified plant, plant cell, protoplast, or plant tissue expressing a recombinant protein of interest, according to the method as previously mentioned, and
isolating and optionally purifying said plant produced protein from said genetically modified plant, plant cell, protoplast, or plant tissue.

The present invention also encompasses a nucleic acid construct as previously defined, notably binary plasmid vector, typically wherein the binary plasmid vector comprises a selectable marker in the T-DNA region and a selectable marker outside the T-DNA region.

The present invention also encompasses a bacterial strain comprising the above defined nucleic acid construct.

The present invention further encompasses a genetically modified plant, plant cell, protoplast, or plant tissue from the Brassica genus expressing a nucleic acid construct as previously defined, or transformed with the bacterial strain above defined, or obtained according the method herein described.

The present invention also relates to a plant-produced protein or polypeptide obtained by the herein described method, for use in therapy, optionally for use in immunotherapy, optionally wherein said plant-produced protein is in the form of a pharmaceutical composition.

### FIGURES

FIG. 1 Synthetic construct of antibody. is a schematic representation of a synthetic gene construct inserted to express PD-1, Nivolumab in Brassica. In total three Nivolumab synthetic constructs were produced. CaMV35S=Cauliflower mosaic virus promoter; NOS=Nospaline synthase; * HIS 6× tag=Polyhistidine-tag; ** HA tag=Human influenza hemagglutinin derived tag.

FIG. 2 Description of the Plasmids. Is a diagrammatic representation of the binary plasmids VB210429. The plasmids were synthesized. A. CaMV35S-GFP, B. CaMV35S-Nivolumab (Codon Optimized for Arabidopsis), C. CaMV35S-Nivolumab (original sequence), D. NOS-Nivolumab (original sequence). Selected restriction endonuclease sites (Ascl, Pad, Pmel) are marked. Abbreviations include LB=Left Border from A. tumefaciens Ti plasmid, RB=Right border region from A. tumefaciens Ti plasmid, KAN=Kanamycin; Hygo=Hygromycin. Refer to Table 1 for a description of gene elements.

FIG. 3 Gel digest. Is a photographic representation showing restriction digestion of cloned synthetic gene construct into the plasmid, VB210429. The plasmids were run on 1% agarose gel. A. CaMV35S-GFP, B. CaMV35S-Nivolumab (Codon Optimized for Arabidopsis), C. CaMV35S-Nivolumab (original sequence), D. NOS-Nivolumab (original sequence). Paired restriction enzymes used are ApaLI+AflII; ApaLI+XbaI; ApaLI+NcoI. M=Marker, GeneRuler 1 Kb DNA Ladder; P=Undigested Plasmid; D=Digested Plasmid.

FIG. 4 Dot Blots showing antibodies. Is a photographic representation showing immunodetection of HIS and HA tag expression in Brassica leaves. Top row: Genetically modified Brassica; Bottom row: Agrifilteration of adult Brassica leaves. Colum 1-4: CaMV35S-GFP, CaMV35S-Nivolumab (Codon Optimized for Arabidopsis), CaMV35S-Nivolumab, NOS-Nivolumab (Left to Right).

FIG. 5 GMO plants. Is a photographic representation showing GMO Brassica plants. A. Wild-type Brassica plant (Control) at age 11 days of germination (DAG). B. Wild-type Brassica plant (Control) at age 40 DAG. C. GMO Brassica at 11 DAG. D. GMO Brassica at 37 DAG.

FIG. 6 Quantity of Antibody. A. Dot Blots detecting tagged antibody with 6×HIS Tag and Actin in GMO Brassica plants. B. Graph measuring chemiluminescence signal intensity. These graphs allow quantification and ratio comparison between signal intensity.

FIG. 7 Immunoprecipitation (IP) of Recombinant PD-1 with PiB001. A. Western blot demonstrating binding of mammalian cell produced recombinant PD-1 antigen to PiB001. Dynabeads treated with PiB001 without PD-1 does not reveal a signal with anti-PD-1 antibody (negative control). However, Dynabead treated with PiB001 and PD-1 reveals a signal for PD-1 (Arrow). B. IP using Protein G demonstrating binding and pulldown of recombinant PD-1 followed by a Western blot with anti-PD-1 antibody. These results show that PiB001 binds too both protein A/G and is able to induce the IP of its recombinant antigen.

FIG. 8 Immunoprecipitation (IP) of PD-1 from human lung tumor. A. IP of PD-1 from human lung tumor lysates demonstrating binding and pulldown of PD-1 followed by a Western blot using anti-PD-1 antibody. B. Western blot of Human Fc demonstrating presence of human Fc binding to protein A. These results show that PiB001 binds to PD-1 in primary patient tumor lysate and to recombinant PD-1.

### DETAILED DESCRIPTION OF THE INVENTION

In the present application, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless specifically mentioned, the invention encompasses any combinations of the various embodiments described herein.

### Definitions

As used herein, the term “construct” is used in reference to nucleic acid molecules that transfer DNA segment(s) from one cell to another. The term “vector” is sometimes used interchangeably with “construct”. The term “construct” includes circular nucleic acid constructs such as plasmid constructs, phagemid constructs, cosmid vectors, etc., as well as linear nucleic acid constructs including, but not limited to, PCR products. As used herein the term “plant nucleic acid expression construct” or “plant nucleic acid construct” refers to a nucleic acid construct including a coding nucleic acid sequence (also named herein “nucleic acid of interest” or “transgene”), which is operably linked to at least one promoter for directing transcription of nucleic acid in a host plant cell and which typically forms the expression cassette.

The term “synthetic construct” as used herein refers to a non-naturally occurring nucleic acid molecule as above defined. Typically, as herein intended the synthetic construct is de novo, or chemically synthesized.

As used herein the term “nucleic acid sequence” refers to a single or double stranded nucleic acid sequence which is isolated and provided in the form of an RNA sequence, a DNA sequence, a complementary polynucleotide sequence (cDNA), a genomic polynucleotide sequence and/or a composite polynucleotide sequences (e.g., a combination of the above).

The term “expression construct” as used herein refers to an expression module or expression cassette made up of a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter and a ribosome binding site, often along with other sequences. Eukaryotic cells are known to utilize promoters, enhancers, and termination and polyadenylation signals.

The term “vector” as used herein refers to a DNA or RNA molecule capable of replication in a host cell and/or to which another DNA or RNA segment can be operatively linked so as to bring about replication of the attached segment. A plasmid is an exemplary vector. Plasmids are the most-commonly used bacterial cloning vectors. These cloning vectors typically contain a site that allows DNA fragments to be inserted, for example a multiple cloning site or polylinker which has several commonly used restriction sites to which DNA fragments may be ligated.

After the gene of interest is inserted, the plasmids are introduced into bacteria by a process called transformation. These plasmids also typically contain a selectable marker, usually an antibiotic resistance gene, which confers on the transformed bacteria an ability to survive and proliferate in a selective growth medium containing the particular antibiotics. The cells after transformation are exposed to the selective media, and only cells containing the plasmid may survive.

As used herein, the term “protein-coding nucleic acid molecule” refers to a nucleic acid molecule or sequence comprising a nucleic acid sequence (typically a DNA sequence) that encodes a protein. A protein-coding DNA molecule may be operably linked to a heterologous promoter in a DNA construct for use in expressing the protein in a cell transformed with, and thus comprising, the recombinant DNA molecule or a portion thereof.

As used herein, “operably linked” refers to the joining of nucleic acid sequences such that one sequence can provide a required function to a linked sequence. In the context of a promoter, “operably linked” means that the promoter is connected to a sequence of interest such that the transcription of that sequence of interest is controlled and regulated by that promoter. When the sequence of interest encodes a protein and when expression of that protein is desired, “operably linked” means that the promoter is linked to the sequence in such a way that the resulting transcript will be efficiently translated. If the linkage of the promoter to the coding sequence is a transcriptional fusion and expression of the encoded protein is desired, the linkage is made so that the first translational initiation codon in the resulting transcript is the initiation codon of the coding sequence. Alternatively, if the linkage of the promoter to the coding sequence is a translational fusion and expression of the encoded protein is desired, the linkage is made so that the first translational initiation codon contained in the 5′ untranslated sequence associated with the promoter and the coding sequence is linked such that the resulting translation product is in frame with the translational open reading frame that encodes the desired protein. Nucleic acid sequences that can be operably linked include, but are not limited to, sequences that provide gene expression functions (e.g., gene expression elements such as promoters, 5′ untranslated regions, introns, protein coding regions, 3′ untranslated regions, polyadenylation sites, and/or transcriptional terminators), sequences that provide DNA transfer and/or integration functions (e.g., T-DNA border sequences, site specific recombinase recognition sites, integrase recognition sites), sequences that provide for selective functions (e.g., antibiotic resistance markers, biosynthetic genes), sequences that provide scorable marker functions (e.g., reporter genes), sequences that facilitate in vitro or in vivo manipulations of the sequences (e.g., polylinker sequences, site specific recombination sequences) and sequences that provide replication functions (e.g., bacterial origins of replication, autonomous replication sequences, centromeric sequences).

As used herein, “transgene expression”, “expressing a transgene”, “protein expression”, and “expressing a protein” mean the production of a protein through the process of transcribing a DNA molecule into messenger RNA (mRNA) and translating the mRNA into polypeptide chains, which may or may not be ultimately folded into proteins.

The term “transformation” as used herein refers to a process of introducing an exogenous nucleic acid (typically DNA) sequence (e.g., a vector, a recombinant DNA molecule) into a plant cell or protoplast in which that exogenous DNA is incorporated into the plant genome or is capable of autonomous replication.

The phrase “transgenic plant” refers to a plant or progeny thereof derived from a transformed plant cell, protoplast, or other transformed plant tissue wherein the plant DNA contains an introduced exogenous DNA molecule not originally present in a corresponding native, non-transgenic plant of the same species.

The terms “sequence-specific recombinase” and “site-specific recombinase” refer to enzymes or recombinases that recognize and bind to a short nucleic acid site or “sequence-specific recombinase target site”, i.e., a recombinase recognition site, and catalyze the recombination of nucleic acid in relation to these sites. These enzymes include recombinases, transposases and integrases.

The term “intron” as used herein refers to a domain of a vector produced by the subject methods that is flanked on the 5′ end by a splice donor site and on the 3′ end by a splice acceptor site, where under appropriate conditions the intron is spliced out of or removed from an mRNA sequence expressed from the vector in which it is present.

The terms “polylinker” or “multiple cloning site” refer to a cluster of restriction enzyme sites, typically unique sites, on a nucleic acid construct that can be utilized for the insertion and/or excision of nucleic acid sequences, such as the coding region of a gene, loxP sites, etc.

The term “termination sequence” refers to a nucleic acid sequence which is recognized by the polymerase of a host cell and results in the termination of transcription. Prokaryotic termination sequences commonly comprise a GC-rich region that has a two-fold symmetry followed by an AT-rich sequence. A commonly used termination sequence is the T7 termination sequence. A variety of termination sequences are known in the art and may be employed in the nucleic acid constructs of the present invention, including the TINT3, TL13, TL2, TR1, TR2, and T6S termination signals derived from the bacteriophage lambda, and termination signals derived from bacterial genes, such as the trp gene of E. coli.

The terms “polyadenylation sequence” (also referred to as a “poly A⁺ site” or “poly A⁺ sequence”) as used herein refers to a DNA sequence which directs both the termination and polyadenylation of the nascent RNA transcript. Efficient polyadenylation of the recombinant transcript is desirable, as transcripts lacking a poly A⁺ tail are typically unstable and rapidly degraded. The poly A⁺ signal utilized in an expression vector may be “heterologous” or “endogenous”. An endogenous poly A⁺ signal is one that is found naturally at the 3′ end of the coding region of a given gene in the genome. A heterologous poly A⁺ signal is one which is isolated from one gene and placed 3′ of another gene, e.g., coding sequence for a protein. Typical examples of well-suited polyA+ termination sequences include the Nopaline synthase (Nos) polyadenylation signal and the cauliflower mosaic virus 35S polyadenylation signal.

As used herein, the terms “selectable marker” or “selectable marker gene” refer to a gene which encodes an enzymatic activity and confers the ability to grow in medium lacking what would otherwise be an essential nutrient; in addition, a selectable marker may confer upon the cell in which the selectable marker is expressed, resistance to an antibiotic or drug. A selectable marker may be used to confer a particular phenotype upon a host cell. When a host cell must express a selectable marker to grow in selective medium, the marker is said to be a positive selectable marker (e.g., antibiotic resistance genes which confer the ability to grow in the presence of the appropriate antibiotic). Selectable markers can also be used to select against host cells containing a particular gene; selectable markers used in this manner are referred to as negative selectable markers.

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

The term “target protein (or polypeptide)” used herein refers to a protein or a polypeptide to be produced in a genetically plant, plant cell, protoplast or plant tissue by a genetic engineering method according to the present invention, and the present invention is not particularly limited thereto. Typically, proteins required to be mass-produced are included.

As used herein, the term “protein” refers to a chain of amino acids linked by peptide (amide) bonds and includes both polypeptide chains that are folded or arranged in a biologically functional way and polypeptide chains that are not. A “sequence” means a sequential arrangement of nucleotides or amino acids. The boundaries of a protein-coding sequence are usually determined by a translation start codon at the 5′-terminus and a translation stop codon at the 3′-terminus.

As used herein the term “multimeric protein” refers to a protein complex comprising two or more separate polypeptide or protein chains associated with each other by non-covalent protein-protein interactions. Said two or more polypeptide or protein chains may identical (in homo (multimeric) proteins) or different (in hetero (multimeric) proteins). In some embodiments, the multimeric protein contains between 30 amino acids and 2000 amino acids, preferably between 50 amino acids and 2000 amino acids. In some embodiment, the multimeric protein contains between 500 and 2000 amino acids, preferably between 700 and 2000 amino acids, preferably between 1000 and 2000 amino acids. In some embodiment, the multimeric protein preferably contains more than 500 amino acids, preferably more than 700 amino acids, preferably more than 1000 amino acids and preferably less than 2000 amino acids. In some embodiments, the multimeric protein is an antibody or a fragment thereof as defined below.

A nucleic acid construct can be stably or transiently introduced into plant cells. In stable transformation, the nucleic acid is integrated into the plant genome and as such it represents a stable and inherited trait. In transient transformation, the exogenous polynucleotide is expressed by the transformed cell, but it is not integrated into the genome and as such it represents a transient trait.

As used herein, the term “host” is typically meant herein to include not only prokaryotes, but also eukaryotes, such as plant cells. A recombinant DNA molecule or gene can be used to transform a host using any of the techniques commonly known to those of ordinary skill in the art.

As used herein, the terms “restriction endonucleases” and “restriction enzymes” refer to bacterial enzymes, each of which cut double-stranded DNA at or near a specific nucleotide sequence.

As used herein the term “plant-produced” notably qualifies the chemical signature associated with plant expression, including, but not limited to, host cell impurities in the preparation, which comprises the chimeric polypeptide and glycosylation patterns on the chimeric polypeptide per se.

The term “plant” as used herein encompasses whole plants, ancestors and progeny of the plants and plant parts, including seeds, leaves, shoots, stems, roots (including tubers), plant cells, protoplasts, tissues and organs. The plant may be in any form including suspension cultures, embryos, meristematic regions, callus tissue, leaves, gametophytes, sporophytes, pollen, and microspores.

The term “plants” as used herein notably include all plants which belong to the superfamily Viridiplantae, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp., Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp., Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp., Dolichos spp., Dorycnium rectum, Echinochloa pyramidalis, Ehraffia spp., Eleusine coracana, Eragrestis spp., Erythrina spp., Eucalyptus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, maize, wheat, barely, rye, oat, peanut, pea, lentil and alfalfa, cotton, rapeseed, canola, pepper, sunflower, tobacco, eggplant, Eucalyptus, a tree, an ornamental plant, a perennial grass and a forage crop. Alternatively, algae and other non-Viridiplantae can be used for the methods of the present invention.

The Brassicaceae plant family is commonly known as the mustard family, Brassicaceae contains some 338 genera and more than 3,700 species of flowering plants. Brassicaceae species are characterized by four-petalled cross-shaped flowers that feature two long and two short stamens and produce pod like fruits known as siliques. Among the Brassicaceae family, cabbage plants and relative, from the Brassica genus, are of particular relevance.

Brassica are leafy green, red (purple), or white (pale green) plants within the Brassicaceae family. Brassica plants are commonly grown in agriculture and horticulture as a food source because of their dense-leaved heads. Most of the Brassica genus plants are annual or biennials. Some of the commonly recognized vegetables within this genus are mustards, Canola, Broccoli, Cauliflower, Cabbages, Choy Sum, and Brussels sprouts. Generally grown cabbages weigh within a range of 500 to 1000 grams. They are cultivated in well-drained soils with a pH between 6.0 & 8, receiving full sun. These growing conditions allow the plants to develop densely leaved heads. The plants require adequate levels of phosphorus, potassium, and nitrogen in the soil, especially during the early stages of growth. These plants grow best between 4 and 24° C. A temperature variation may cause the plant to blot and produce flowers. In some embodiments, lower temperatures may cause vernalization of the flowers. Hence, brassica can be planted at the beginning of the cold period and survive until a later warm period before inducing flowers.

Cabbage plants notably include bok choy (Brassica rapa, variety Chinensis), brown mustard (Brassica juncea), broccoli (Brassica oleracea, variety italica), Brussels sprouts (Brassica oleracea, variety gemmifera), cabbage (Brassica oleracea, variety capitata), cauliflower (Brassica oleracea, variety botrytis), collard (Brassica oleracea, variety acephala), kale (Brassica oleracea, variety acephala), kohlrabi (Brassica oleracea, variety gongylodes), napa cabbage (Brassica rapa, variety pekinensis), rape (Brassica napus, variety napus), rutabaga (Brassica napus, variety napobrassica), and turnip (Brassica rapa, variety rapa).

Other well-suited plants according to the present invention include plants from the Spinacia genus (which most common member is spinach), a flowering plant genus in the subfamily Chenopodioideae of the family Amaranthaceae, and plants from the Lactuca genus (which best-known representative is the garden lettuce or Lactuca sativa) that are flowering plants in the daisy family, Asteraceae.

The term “antibody” as used herein refers to immunoglobulin molecules and immunologically active portions of immunoglobulin molecules, i.e., molecules that contain an antigen binding site that immunospecifically binds to an antigen. As such, the term antibody encompasses not only whole antibody molecules, but also antibody fragments as well as variants (including derivatives) of antibodies. In some embodiments, whole antibody molecules comprise between 500 to 1500 amino acids, more preferably comprise between 1200 and 1500 amino acids. In natural antibodies of rodents and primates, two heavy chains are linked to each other by disulfide bonds, and each heavy chain is linked to a light chain by a disulfide bond. There are two types of light chains, lambda (2) and kappa (κ). There are five main heavy chain classes (or isotypes) which determine the functional activity of an antibody molecule: IgM, IgD, IgG, IgA and IgE. In humans there are four subclasses of IgG: IgG1, IgG2, IgG3 and IgG4 (numbered in order of decreasing concentration in serum). IgA exists in two subclasses, IgA1 and IgA2. Both IgA1 and IgA2 have been found in external secretions (secretory IgA), where IgA2 is more prominent than in the blood (serum IgA). Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). Secretory IgA are polymeric:

2-4 IgA monomers are linked by two additional chains: the immunoglobulin joining (J) chain(s) and secretory component (SC). The J chain binds covalently to two IgA molecules through disulfide bonds between cysteine residues. The secretory component is a proteolytic cleavage product of the extracellular part of the polymeric immunoglobulin receptor (pIgR) which binds to J-chain containing polymeric Ig. Polymeric IgA (mainly the secretory dimer) is produced by plasma cells in the lamina propria adjacent to mucosal surfaces. It binds to the polymetic immunoglobulin receptor on the basolateral surface of epithelial cells, and is taken up into the cell via endocytosis. The receptor-IgA complex passes through the cellular compartments before being secreted on the luminal surface of the epithelial cells, still attached to the receptor. Proteolysis of the receptor occurs, and the dimeric IgA molecule, along with a portion of the receptor known as the secretory component-known as sIgA, are free to diffuse throughout the lumen.

The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) can participate in the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system. The predicted CDRs of some anti-SARS-COV-2 antibodies, such as Cv2.1169, Cv2.5213, Cv2.3235, Cv2.1353 and Cv2.3194 are described herein.

The term “monoclonal antibody” as used herein refers to a preparation of antibody molecules of single specificity. A monoclonal antibody displays a single binding specificity and affinity for a particular epitope. Accordingly, the term “human monoclonal antibody” refers to an antibody displaying a single binding specificity which has variable and constant regions derived from or based on human germline immunoglobulin sequences or derived from completely synthetic sequences. The method of preparing the monoclonal antibody is not relevant for the binding specificity.

As used herein, the term “recombinant antibody” refers to antibodies which are produced, expressed, generated or isolated by recombinant means, such as typically antibodies which are expressed using a recombinant expression vector transfected into a host cell or antibodies isolated from an animal (e.g. a mouse) or a plant which is transgenic due to human immunoglobulin genes; or antibodies which are produced, expressed, generated or isolated in any other way in which particular immunoglobulin gene sequences (such as human immunoglobulin gene sequences) are assembled with other DNA sequences. Recombinant antibodies include, for example, chimeric and humanized antibodies. In some embodiments a recombinant human antibody of this invention has the same amino acid sequence as a naturally-occurring human antibody but differs structurally from the naturally occurring human antibody. For example, in some embodiments the glycosylation pattern is different as a result of the recombinant production of the recombinant human antibody. In some embodiments the recombinant human antibody is chemically modified by addition or subtraction of at least one covalent chemical bond relative to the structure of the human antibody that occurs naturally in humans.

The term “antigen-binding fragment” of an antibody (or simply “antibody fragment”), as used herein, refers to full length or one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment” of an antibody include a Fab fragment, a monovalent fragment consisting of the VL, VH, CL and CH1 domains; a F (ab) 2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989 Nature 341:544-546), which consists of a VH domain, or any fusion proteins comprising such antigen-binding fragments. Furthermore, although the two domains of the Fv fragment, VL and VH, are naturally coded for by separate genes, their coding sequences are typically joint according to the present invention, using chemical DNA synthesis to form a synthetic nucleic acid. In some embodiment, the synthetic nucleic acid encoding VL and CH domains can include a synthetic linker sequence that enables them to be made as a single chain protein in which the VL and VH regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding fragment” of an antibody.

Antigen binding fragments of an antibody that contain the variable domains comprising the CDRs domains are typically selected from Fv, dsFv, scFv, Fab, Fab′, F(ab′)2. The F(ab′)2 fragment can be produced by pepsin digestion of an antibody below the hinge disulfide; it comprises two Fab′ fragments, and additionally a portion of the hinge region of the immunoglobulin molecule. Fab fragments are monomeric fragments obtainable by papain digestion of an antibody; they comprise the entire L chain, and a VH-CH1 fragment of the H chain, bound together through a disulfide bond. The Fab′ fragments are obtainable from F(ab′)2 fragments by cutting a disulfide bond in the hinge region. F(ab′)2 fragments are divalent, i.e. they comprise two antigen binding sites, like the native immunoglobulin molecule; on the other hand, Fv (a VHVL dimer constituting the variable part of Fab), dsFv, scFv, Fab, and Fab′ fragments are monovalent, i.e. they comprise a single antigen-binding site. These basic antigen-binding fragments can be further combined together to obtain multivalent antigen-binding fragments, such as diabodies, tribodies or tetrabodies. These multivalent antigen-binding fragments are also part of the present invention as they can also be produced in a method of the present. Fv fragments consist of the VL and VH domains of an antibody associated together by hydrophobic interactions; in dsFv fragments, the VH: VL heterodimer is stabilized by a disulphide bond; in scFv fragments, the VL and VH domains are connected to one another via a flexible peptide linker thus forming a single-chain protein.

The expression “variable domain” or “variable region” of an antibody heavy or light chain are used interchangeably as the variable region of an antibody consists of a variable domain.

The phrases “an antibody recognizing an antigen (X)”, “an antibody having specificity for an antigen (X)”, “an anti-X antibody”, “an antibody against X”, and an “antibody directed against” are used interchangeably herein with the term “an antibody which binds specifically to an antigen (X)”.

The variable regions of the antibody as described above may be associated with antibody constant regions, like IgA, IgM, IgE, IgD or IgG such as IGg1, IgG2, IgG3, IgG4. Said variable regions of the antibody is preferably associated with IgG or IgA constant region; preferably IgG1 or IgA (IgA1, IgA2) constant regions. These constant regions may be further mutated or modified, by methods known in the art, in particular for modifying their binding capability towards Fc receptor or enhancing antibody half-life. The antibody comprising IgA constant region may further comprise a J chain and/or a secretory component to generate a polymeric or secretory IgA.

As used herein, the term “IgG Fc region” is used to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody. The numbering of residues in the Fc region is that of the EU index of Kabat.

A biosimilar monoclonal antibody (mAb) as used herein refers according to the WHO's definition (see https://www.who.int/biologicals/biotherapeutics/WHO_TRS_1004_web_Annex 2.pdf?ua=1) to a mAb product that has is almost an identical copy of an original/reference mAb and that is similar in terms of quality, safety and efficacy to said already licensed reference mAb.

As used herein, the percent identity between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences or nucleotide sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. Alternatively, the percent identity between two amino acid sequences or nucleotide sequences can be determined using the Needleman and Wunsch (J. Mol, Biol. 48:444-453, 1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6.

The percent identity between two nucleotide or amino acid sequences may also be determined using for example algorithms such as the BLASTN program for nucleic acid or amino acid sequences using as defaults a word length (W) of 11, an expectation (E) of 10, M=5, N=4, and a comparison of both strands.

As used herein the term “protein tag” or “molecular tag” refers to peptide sequences which are genetically grafted onto a recombinant protein. They can be removed by chemical agents or by enzymatic means, such as proteolysis or intein splicing. They can be added to either end of the target protein, so they are either C-terminus or N-terminus specific or are both C-terminus and N-terminus specific (and are therefore added respectively in 3′ or 5′ of the protein coding sequence). Some tags are also inserted into the coding sequence of the protein of interest; they are known as internal tags. Molecular tag(s) can be selected based on their molecular size, steric hindrance, intended use such as the purification process, etc. Molecular tags notably include:
Affinity tags usable in purification methods (typically from crude biological source) using an affinity technique. These include chitin binding protein (CBP), maltose binding protein (MBP), Strep-tag, poly(His) tag and glutathione-S-transferase (GST). The poly(His) (5-10 histidines bound by a nickel or cobalt chelate) tag is a widely used protein tag, which binds to metal matrices.
Solubilization tags typically used to assist in the proper folding in proteins and keep them from precipitating. These include thioredoxin (TRX) and poly (NANP), MBP, and GST.
Chromatography tags usable to alter chromatographic properties of a protein to afford different resolution across a particular separation technique. These typically include polyanionic amino acids, such as FLAG-tag.
Epitope tags are short peptide sequences usually derived from viral genes and usable for western blotting, immunofluorescence, antibody purification and immunoprecipitation experiments. Epitope tags include ALFA-tag, V5-tag, Myc-tag, HA-tag, Spot-tag, T7-tag and NE-tag.
Fluorescence tags which give visual readout on a protein typically include GFP and its variants.

Tags are generally removed after purification, for example by using specific proteolysis. While some common tags such as SUMO and FLAG, are cleaved by specific proteases, addition of one or more protease cleavage recognition sites can be envisioned in some embodiments. Typical proteases include for example TEV protease, Thrombin, Factor Xa or Enteropeptidases, SUMO proteases).

A classical list of well-known tag include as a matter of example: ALFA-tag (de novo designed helical peptide tag), AviTag, C-tag (that binds to a single-domain camelid antibody), Calmodulin-tag (bound by the protein calmodulin), GST, polyglutamate tag, polyarginine tag, E-tag, FLAG-tag, HA-tag (a peptide from hemagglutinin recognized by an antibody), His-tag (5-10 histidines bound by a nickel or cobalt chelate), HBH, MBP, Myc-tag (a peptide derived from c-myc recognized by an antibody), NE-tag (a 18-amino-acid synthetic peptide recognized by a monoclonal IgG1 antibody), Rho1D4-tag, (refers to the last 9 amino acids of the intracellular C-terminus of bovine rhodopsin), S-tag (derived from Ribonuclease A, SBP-tag (which binds to streptavidin), Softag 1, Softag 3, Spot-tag (recognized by a nanobody), Strep-tag (which binds to streptavidin or the modified streptavidin called streptactin), SUMO, T7-tag (epitope tag derived from the T7 major capsid protein of the T7 gene), TAP, TRX, TC tag (tetracysteine tag recognized by FlAsH and ReAsH biarsenical compounds), Ty tag, V5 tag (recognized by an antibody), VSV-tag (recognized by an antibody), Xpress tag. Well-suited tags according to the present invention notably include for example HA tag and poly(His) tag.

### Method for Producing a Genetically Modified Plant, Plant Cell, Protoplast, or Plant Tissue

The present invention relates to a method for producing a genetically modified plant (also named herein genetically modified organism, or GMO), plant cell, protoplast or plant tissue expressing a recombinant protein of interest, wherein
said method comprises the introduction into the plant, plant cell, protoplast or plant tissue of a nucleic acid construct that provides for stable expression of the protein of interest into the plant, plant cell, protoplast, or plant tissue;
wherein:
   the plant is selected from the Spinacia genus, the Lactuca genus and the Brassica genus, and
   the nucleic acid construct is a single synthetic construct comprising a regulatory sequence active in a plant for the expression of an operably linked protein-coding nucleic molecule, and a 3′ untranslated region (3′UTR region). This feature means more particularly that only one expression vector (typically a plasmid) is used for the stable recombinant expression of a target protein in a plant. This is especially advantageous in the case of large multimeric proteins such as antibodies.

### Plants

Typically, the plant is a leafy plant.

In some embodiments, the plant is a carnation plant and in particular a plant having spray carnations exhibiting altered inflorescence. The altered inflorescence may be in any tissue or organelle including flowers, petals, anthers and styles. Particular inflorescence contemplated herein includes a color in the range of red-purple to blue color such as a purple to blue color. The color determination is conveniently measured against the Royal Horticultural Society (RHS) color chart (RHSCC) and includes colors 8 1A, 86A, 87A and colors in between or proximal to either end of the above range. The term “inflorescence” is not to be narrowly construed and relates to any colored cells, tissues organelles or parts thereof, as well as flowers and petals.

Advantageously, the plant is an edible plant.

In preferred embodiments, the plant is from the Brassica genus. Well-suited plants from the Brassica genus according to the present invention include typically: Canola, Broccoli, Cauliflower, Cabbages, Choy Sum, and Brussels sprouts. Generally grown cabbages weigh within a range of 500 to 1000 grams. They are cultivated in well-drained soils with a pH between 6.0 & 8, receiving full sun. These growing conditions allow the plants to develop densely leafed heads. The plants require adequate levels of phosphorus, potassium, and nitrogen in the soil, especially during the early stages of growth. These plants grow best between 4 and 24° C. A temperature variation may cause the plant to blot and produce flowers. In some embodiments, lower temperatures may cause vernalization of the flowers. Hence, brassica can be planted at the beginning of the cold period and survive until a later warm period before inducing flowers.

In some embodiments, the plant from the Brassica genus is a cabbage plant, preferably a cabbage plant selected from bok choy (Brassica rapa, variety chinensis), brown mustard (Brassica juncea), broccoli (Brassica oleracea, variety italica), Brussels sprouts (Brassica oleracea, variety gemmifera), cabbage (Brassica oleracea, variety capitata), cauliflower (Brassica oleracea, variety botrytis), collard (Brassica oleracea, variety acephala), kale (Brassica oleracea, variety acephala), kohlrabi (Brassica oleracea, variety gongylodes), napa cabbage (Brassica rapa, variety pekinensis), rape (Brassica napus, variety napus), rutabaga (Brassica napus, variety napobrassica), and turnip (Brassica rapa, variety rapa).

In some embodiments, the plant from the Brassica genus is selected from the Brassica oleracea species e.g. is selected from cabbage (Brassica oleracea, variety capitata), broccoli (Brassica oleracea, variety italica), Brussels sprouts (Brassica oleracea, variety gemmifera), cauliflower (Brassica oleracea, variety botrytis), collard (Brassica oleracea, variety acephala), kale (Brassica oleracea, variety acephala), and kohlrabi (Brassica oleracea, variety gongylodes); the Brassica rapa species eg. is selected from bok choy (Brassica rapa, variety chinensis), napa cabbage (Brassica rapa, variety pekinensis), and turnip (Brassica rapa, variety rapa); and the Brassica napus species e.g. is selected from rape (Brassica napus, variety napus) and rutabaga (Brassica napus, variety napobrassica).

In some embodiments, the plant from the Brassica genus is selected from the Brassica oleracea species e.g. is cabbage (Brassica oleracea, variety capitata).

In some embodiments, the plant from Brassica genus is cabbage.

In some embodiments the plant is not selected from the Lactuca genus, typically the plant is not a lettuce.

The method of the present invention involves stable transformation of the plant, parts or derivatives thereof with a nucleic acid construct as herein defined which is targeted to and inserted in either the nuclear or chloroplast genome. Stable transformation characteristics include (1) a heritable transgene, permitting establishment of seed stock for future use, and (2) protein production scalable to field production. Stable transformation is also typically easily detected by inserting a selectable marker in the nucleic acid construct and selecting through artificial selection on media. Preferably, stable transformation of a plant according to the present invention allows whole plant expression of the nucleic acid of interest (i.e., transgene), notably in various plant tissues including or consisting of leaves, stem, seeds and/or roots.

### Nucleic Acid (DNA) Constructs Comprising Plant Expression Cassette Construct

The construction of expression cassettes, for use in monocotyledonous or dicotyledonous plants, is well established. Expression cassettes are nucleic acid constructs comprising promoter, coding, and polyadenylation sequences which are operably linked.

According to the present invention, the nucleic acid construct is chemically synthesized (i.e., de novo synthesized). Typically, synthetic DNA (or nucleic acid) constructs are designed and manipulated using computer-aided design software. The designed DNA is then divided into synthesizable pieces (synthons) up to 1-1.5 kbp. The synthons are then broken up into overlapping single-stranded oligonucleotide sequences and chemically synthesized. The oligonucleotides are then assembled together into the designed synthons using gene synthesis techniques. If necessary, multiple synthons can be assembled together into larger DNA assemblies or devices. The assembled DNAs are then typically cloned into an expression vector and sequence-verified. The techniques and technologies that enable the synthesis of DNA oligonucleotides have been recently reviewed in Hughes R A, Ellington A D. Synthetic DNA Synthesis and Assembly: Putting the Synthetic in Synthetic Biology. Cold Spring Harb Perspect Biol. 2017; 9 (1): a023812.

The nucleic acid of interest can code for any protein or interest (or polypeptide). Proteins (or polypeptides) of interest according to the present invention are however multimeric proteins (or polypeptides), and notably antibodies. In some embodiments of the present invention, the plant-produced antibody is an antibody for use in cancer therapy, against infectious diseases or in auto-immune disease's therapy. In some embodiments, the antibody can be directed against bacterial, viral, fungal or cancer antigens. Cancer antigens are antigens expressed in the context of a cancer that can be targeted for cancer therapy. In some embodiments, the antibody is directed against a tumor antigen, or an immune checkpoint molecule (in particular an inhibitory immune checkpoint molecule). Examples of relevant immune checkpoint molecules include PD1, PDL1, CTLA4, LAG3, BTLA, OX2R, TIM-3, TIGIT, LAIR-1, PGE2 receptor, EP2/4 adenosine receptor, or A2AR. Typically, the antibody is an anti-PD-1.

In some embodiments, the nucleic acid of interest codes for a multimeric protein having a molecular weight comprised between 20 kDa and 170 kDa, between 20 kDa and 150 kDa, between 60 kDa and 170 kDa, or between 60 kDa and 150 kDa.

In some embodiments, the nucleic acid of interest codes for a multimeric protein, for example an antibody fragment, having a molecular weight comprised between 20 and 130 kDa, between 25 and 130 kDa, between 20 and 100 kDa, between 25 and 100 kDa, between 20 and 70 kDa or between 25 and 70 kDa.

In some embodiments, the nucleic acid of interest codes for a multimeric protein, for example an antibody, having a molecular weight comprised between 130 and 170 kDa, preferably between 140 and 160 kDa, more preferably between 145 and 150 kDa.

In some embodiments, the antibody is a biosimilar antibody for a reference approved monoclonal antibody, notably a biosimilar for Nivolumab.

In some embodiments, the nucleic acid coding for the protein of interest comprises nucleic acid sequences coding for VL and VH sequences having at least, 80, 85, 90; 91, 92; 93; 94; 95; 96; 97; 98; 99 and 100% identity respectively to the VL and VH sequences of SEQ ID NO:1 and 2 or the antigen binding fragment thereof, or with the CDRs portion thereof.

Although both VH and VL domains are naturally encoded by different gene, in the de novo synthesized coding nucleic acid of the present invention, the nucleic acid sequences coding for the VH and VL domains are typically fused to form a single synthetic nucleic acid, such that the whole antibody can be produced from a single nucleic acid construct.

In some embodiments, the nucleic acid coding for the protein of interest (to be produced) include one or more protein tags (also named herein molecular tags) usable for purification of the protein of interest from the plant (notably the plant cell or plant tissue(s) as defined previously). Typically, in embodiments, where a multimeric protein is produced according to a method of the present invention, the nucleic acid construct is synthesized such as a molecular tag is de novo attached to each nucleic acid sequence encoding for a given monomer (i.e., polypeptide chain of the multimeric protein), in particular a different molecular tag is attached to each “monomer-coding” nucleic acid sequence. Typically, the molecular tag is attached to the 3′ end of said sequence(s).

For example, in embodiments, where the protein of interest is an antibody comprising a VH and a VL domain, a molecular tag is attached to each sequence coding respectively for the VH and VL sequences. Typically, the molecular tags are attached to the 3′ end of the VH and VL coding sequences. Typically said molecular tags are different.

The use of tag attached to the nucleic acid coding sequence, and in particular attached to the coding sequence corresponding to each coding sequence of the multimeric protein, is highly advantageous as it may serve two critical functions. First these tags can be used to detect the expression of the recombinant protein. Indeed, the expression level of the protein of interest can easily be assessed using antibodies directed against the selected tag. Furthermore, it allows for double simultaneous purification of the various polypeptide chains of the recombinant multimeric protein. In particular in the case of a recombinant antibody, the use of tags attached to each of the coding sequence of the VH and VL domain allows for double simultaneous purification of the full antibody. This method of purification improves yield and efficiency of purification, while reducing waste and run off. In some embodiments, selected tags (like (poly) histidine tags) are also advantageous in affinity chromatography and in particular in immobilized metal ion affinity chromatography (IMAC). Having more than one tag is advantageous as the sample can be purified sequentially with each tag.

In some embodiment, the synthetic nucleic acid of interest comprises nucleic acid sequences coding for VL and VH sequences having respectively at least, 80, 85, 90; 91, 92; 93; 94; 95; 96; 97; 98; 99 and 100% identity with SEQ ID NO:3, notably with the VL and VH sequences of SEQ ID NO:3.

In some embodiments, the synthetic nucleic acid of interest comprises a nucleic acid sequence coding for an amino acid sequence having at least, 80, 85, 90; 91, 92; 93; 94; 95; 96; 97; 98; 99 and 100% identity with SEQ ID NO:3.

In some embodiments, the synthetic nucleic acid of interest comprises a nucleic acid sequence coding for an amino acid sequence having, 1; 2; 3; 4; 5; 6; 7; 8; 9; 10; 11; 12; 13; 14 or 15 amino acid substitution(s) as compared with SEQ ID NO:3.

In some embodiments, the synthetic nucleic acid of interest comprises a nucleic acid sequence having at least, 80, 85, 90; 91, 92; 93; 94; 95; 96; 97; 98; 99 and 100% identity with SEQ ID NO: 4 or 5.

According to some embodiments of the present invention, the nucleic acid sequences encoding the polypeptides or proteins of the present invention are optimized for expression in plants. Examples of such sequence modifications include, but are not limited to, an altered G/C content to more closely approach that typically found in the plant species of interest, and the removal of codons atypically found in the plant species commonly referred to as codon optimization. In one embodiment, the codon usage of the nucleic acid sequence encoding the polypeptide e.g., chimeric polypeptide or protein, is optimized for Spinacia, Lactuca, or Brassica genus.

The term “codon optimization” refers to the selection of appropriate DNA nucleotides for use within a structural gene or fragment thereof that approaches codon usage within the plant of interest. Therefore, an optimized gene or nucleic acid sequence refers to a gene in which the nucleotide sequence of a native or naturally occurring gene has been modified to use statistically-preferred or statistically-favoured codons within the plant. The nucleotide sequence typically is examined at the DNA level and the coding region optimized for expression in the plant species determined using any suitable procedure, for example as described in Sardana et a1. (1996, Plant Cell Reports 15:677-681). In this method, the standard deviation of codon usage, a measure of codon usage bias, may be calculated by first finding the squared proportional deviation of usage of each codon of the native gene relative to that of highly expressed plant genes, followed by a calculation of the average squared deviation. The formula used is: 1 SDCU=n=1 N[(Xn−Yn)/Yn]2/N, where Xn refers to the frequency of usage of codon n in highly expressed plant genes, where Yn to the frequency of usage of codon n in the gene of interest and N refers to the total number of codons in the gene of interest. A table of codon usage from highly expressed genes of dicotyledonous plants has been compiled using the data of Murray et al. (1989, Nuc Acids Res. 17:477-498).

One method of optimizing the nucleic acid sequence in accordance with the preferred codon usage for a particular plant cell type is based on the direct use, without performing any extra statistical calculations, of codon optimization tables such as those provided on-line at the Codon Usage Database through the NIAS (National Institute of Agrobiological Sciences) DNA bank in Japan (Hypertext Transfer Protocol://World Wide Web (dot) kazusa (dot) or (dot) jp/codon/). The Codon Usage Database contains codon usage tables for a number of different species, with each codon usage table having been statistically determined based on the data present in Genbank.

By using such codon optimization tables to determine the most preferred or most favoured codons for each amino acid in a particular species (for example, rice), a naturally-occurring nucleotide sequence encoding a protein of interest can be codon optimized for that particular plant species. This is achieved by replacing codons that may have a low statistical incidence in the particular species genome with corresponding codons, in regard to an amino acid, that are statistically more favoured. However, one or more less-favoured codons may be selected to delete existing restriction sites, to create new ones at potentially useful junctions (5′ and 3′ ends to add signal peptide or termination cassettes, internal sites that might be used to cut and splice segments together to produce a correct full-length sequence), or to eliminate nucleotide sequences that may negatively affect mRNA stability or expression.

The desired encoding nucleotide sequence may already, in advance of any modification, contain a number of codons that correspond to a statistically-favoured codon in a particular plant species. Therefore, codon optimization of the native nucleotide sequence may comprise determining which codons, within the desired nucleotide sequence, are not statistically-favoured with regards to a particular plant, and modifying these codons in accordance with a codon usage table of the particular plant to produce a codon optimized derivative. A modified nucleotide sequence may be fully or partially optimized for plant codon usage provided that the protein encoded by the modified nucleotide sequence is produced at a level higher than the protein encoded by the corresponding naturally occurring or native gene. Construction of synthetic genes by altering the codon usage is described in for example PCT Patent Application 93/07278.

Coding nucleic acid molecules of interest, are typically inserted into a nucleic acid construct or vector, typically an expression vector, as part of a construct having the said nucleic acid molecule operably linked to a gene expression element that functions in a plant to affect expression of the protein encoded by the nucleic acid molecule. Methods for constructing nucleic acid constructs and typically vectors are well known in the art.

The components for the expression cassette of a nucleic acid construct, according to the present invention, typically include one or more gene expression elements operably linked to a transcribable nucleic acid (typically DNA) sequence, such as the following: a promoter for the expression of an operably linked DNA, an operably linked protein-coding nucleic acid molecule, and a 3′ untranslated region (or part(s) thereof). Further gene expression elements useful in practicing the present invention include, but are not limited to, one or more of the following type of elements: 5′ untranslated region or element(s), enhancer, leader, cis-acting element, intron, polyadenylation sequences, signal sequence(s), and/or transcriptional terminators).

Promoters useful in practicing the present invention include those that function in a plant cell for expression of an operably linked polynucleotide. Typically, the promoter is a heterologous promoter. As used herein in the context of a DNA construct, refers to either: i) a promoter that is derived from a source distinct from the operably linked structural coding sequence or ii) a promoter derived the same source as the operably linked structural gene, where the promoter's sequence is modified from its original form. In some embodiments, synthetic promoters that are chemically synthesized rather than biologically derived are used. Usually synthetic promoters incorporate sequence changes that optimize the efficiency of RNA polymerase initiation. Promoters, such as the 35S promoter, can be used as single or multiple copies to increase gene transcription efficiency. Useful promoters for recombinant protein expression in plants are also described in Makhzoum A, Benyammi R, Moustafa K, Trémouillaux-Guiller J. Recent advances on host plants and expression cassettes' structure and function in plant molecular pharming. BioDrugs. 2014; 28 (2): 145-159.

In some embodiments, promoters that are active in certain plant tissues (i.e., tissue specific promoters) can also be used to drive expression of target proteins and peptides. Examples of useful tissue-specific, developmentally regulated promoters include but are not limited to the β-conglycinin 7S promoter (Doyle et al., 1986), seed-specific promoters (Lam and Chua, 1991), and promoters associated with napin, phaseolin, zein, soybean trypsin inhibitor, ACP, stearoyl-ACP desaturase, or oleosin genes. Examples of root specific promoters include but are not limited to the RB7 and RD2 promoters described in U.S. Pat. Nos. 5,459,252 and 5,837,876, respectively.

Plant promoters are varied and well known in the art and include those that are inducible, viral, synthetic, constitutive, inducible, temporally regulated, spatially regulated, and/or spatio-temporally regulated. For example, the promoter can be a viral promoter such as the cauliflower mosaic virus (CAM) 35S and 19S promoters (described in Odell J T, Nagy F, Chua N H. Identification of DNA sequences required for activity of the cauliflower mosaic virus 35S promoter. Nature. 1985; 313 (6005): 810-812; and in Seternes T, Tonheim T C, Myhr A I, Dalmo R A. A plant 35S CaMV promoter induces long-term expression of luciferase in Atlantic salmon. Sci Rep. 2016; 6:25096. Published 2016 Apr. 26), and the FMV (Figwort mosaic virus) 35S promoter. The CaMV35S and FMV35S promoters are active in a variety of transformed plant tissues and most plant organs (e.g., callus, leaf, seed and root). Enhanced or duplicate versions of the CaMV35S and FMV35S promoters can also be used. Other useful promoters include the nopaline synthase (NOS) and octopine synthase (OCS) promoters (which are carried on tumor-inducing plasmids of A. tumefaciens), the cauliflower mosaic virus (CaMV) 19S promoters, a maize ubiquitin promoter, the rice Act1 promoter, and the Figwort Mosaic Virus (FMV) 35S promoter (see, e.g., U.S. Pat. No. 5,463,175).

In some embodiments, inducible promoters may be favored. Inducible promoters include, but are not limited to, promoters induced by heat (e.g., heat shock promoters such as Hsp70), promoters induced by light (e.g., the light-inducible promoter from the small subunit of ribulose 1,5-bisphosphate carboxylase, ssRUBISCO, a very abundant plant polypeptide), promoters induced by cold (e.g., COR promoters), promoters induced by oxidative stress (e.g., catalase promoters), promoters induced by drought (e.g., the wheat Em and rice rab16A promoters), and promoters induced by multiple environmental signals (e.g., rd29A promoters, Glutathione-S-transferase (GST) promoters). Useful promoters induced by fungal infections include those promoters associated with genes involved in phenylpropanoid metabolism (e.g., phenylalanine ammonia lyase, chalcone synthase promoters), genes that modify plant cell walls (e.g., hydroxyproline-rich glycoprotein, Glycine-rich protein, and peroxidase promoters), genes encoding enzymes that degrade fungal cell walls (e.g., chitinase or glucanase promoters), genes encoding thaumatin-like protein promoters, or genes encoding proteins of unknown function that display significant induction upon fungal infection. Maize and flax promoters, designated as Mis1 and Fis1, respectively, are also induced by fungal infections in plants and can be used (U.S. patent Application No. 20020115849)

Temporally regulated promoters are promoters where the rate of RNA polymerase binding and initiation is modulated at a specific time during development. Examples of temporally regulated promoters are given in Benfey P N, Chua N H. Regulated genes in transgenic plants. Science. 1989; 244 (4901): 174-181 . . . . Spatially regulated promoters are promoters where the rate of RNA polymerase binding and initiation is modulated in a specific structure of the organism such as the leaf, stem or root. Examples of spatially regulated promoters are given in Benfey & Chua, 1989. Spatiotemporally regulated promoters are promoters where the rate of RNA polymerase binding and initiation is modulated in a specific structure of the organism at a specific time during development. Typical spatiotemporally regulated promoters include the EPSP synthase-35S promoter described by Benfey & Chua, 1989, and the deacetylvindoline 4-O-acetyl transferase gene promoter.

Polyadenylation sequences can be typically selected from the group consisting of a CaMV35S, a Nopaline synthase polyadenylation signal (NOS), a rice lactate dehydrogenase, and a wheat Hsp17 polyadenylation sequence.

Nucleic acid constructs of the invention can further comprise one or more sequences that encodes a signal peptide that is operably linked to the sequence that encodes the target protein. The signal peptides used in the constructs of the invention are selected from the group consisting of, monocot plant signal peptides, dicot plant signal peptides, bacterial signal peptides, and synthetic signal peptides. These peptides are recognizable by the host system and can increase the level of transcription and translation of recombinant proteins with enhanced stability and protection against protease degradation by targeting recombinant protein in the secretory pathway to specific organelles. Useful peptide sequences include the cereal α-amylase signal peptide, the signal peptide from the 2S2 seed storage protein, the AP24 Osmotin, the bacterial LT-B from E. coli, the sequence KDEL (Lys-Asp-Glu-Leu), encoding for endoplasmic reticulum (ER) retention signal, the ER SEKDEL peptide, the HDEL (His-Asp-Glu-Leu) retention peptide, the prolamin signal peptide, the phaseolin signal peptide (sp) and phaseolin vacuolar sorting-signal (AFVY). Useful peptides for practicing the present invention are typically described in Makhzoum A, Benyammi R, Moustafa K, Trémouillaux-Guiller J. Recent advances on host plants and expression cassettes' structure and function in plant molecular pharming. BioDrugs. 2014; 28 (2): 145-159 (see notably table 4).

Advantageously, in some embodiments, the nucleic acid construct and in particular the expression cassette comprises a nucleic acid sequence coding for a Kozak consensus peptide. Kozak consensus peptide plays an important role in eukaryotic gene expression system (see notably for reference Amani J, Mousavi S L, Rafati S, Salmanian A H. Immunogenicity of a plant-derived edible chimeric EspA, Intimin and Tir of Escherichia coli 0157: H7 in mice. Plant Sci. 2011; 180 (4): 620-627). Kozak and KDEL (vacuole retention peptides) are useful to enhance the expression, stability, and retention of recombinant protein.

In some embodiments, the nucleic acid construct and in particular the expression cassette comprises a constitutive promoter preferably selected from 35CaMVS or NOS, optionally followed by a Kozak consensus sequence, and/or a polyadenylation sequence which is preferably Nopaline synthase polyadenylation signal (NOS).

In some embodiments, the nucleic acid construct and in particular the expression cassette comprises a multimeric protein-coding DNA molecule wherein the nucleic acid sequences coding for the different chains of the multimeric protein are fused to form a single synthetic nucleic acid, optionally wherein a molecular tag is attached to each sequence coding for the different chains of the multimeric protein. Typically, the molecular tags are attached to the 3′ end of each coding sequences. Preferably, the molecular tags are poly(His) tags.

In some embodiments, the nucleic acid construct and in particular the expression cassette comprises an antibody-coding DNA molecule, wherein the nucleic acid sequences coding for the different chains of the antibody are fused to form a single synthetic nucleic acid comprising a nucleic acid sequence coding for an antibody light chain or the antigen-binding fragment thereof and a nucleic acid sequence coding for an antibody heavy chain or the antigen-binding fragment thereof, optionally wherein the antibody is an anti-PD-1, optionally wherein the antibody is nivolumab, optionally wherein a molecular tag is attached to each sequence coding for the antibody light chain and/or the antibody heavy chain or the antigen-binding fragment(s) thereof. Typically, the molecular tags are attached to the 3′ end of each coding sequences. Preferably, the molecular tags are poly(His) tags. In some embodiments, the nucleic acid construct and in particular the expression cassette comprises:
a constitutive promoter preferably selected from 35CaMVS or NOS, optionally followed by a Kozak consensus sequence, and/or a polyadenylation sequence which is preferably Nopaline synthase polyadenylation signal (NOS), and
an antibody-coding DNA molecule, wherein the nucleic acid sequences coding for the different chains of the antibody are fused to form a single synthetic nucleic acid comprising a nucleic acid sequence coding for an antibody light chain or the antigen-binding fragment thereof and a nucleic acid sequence coding for an antibody heavy chain or the antigen-binding fragment thereof, optionally wherein the antibody is an anti-PD-1, optionally wherein the antibody is nivolumab, optionally wherein a molecular tag is attached to each sequence coding for the antibody light chain and/or the antibody heavy chain or the antigen-binding fragment(s) thereof. Typically, the molecular tags are attached to the 3′ end of each coding sequences. Preferably, the molecular tags are poly(His) tags.

In some embodiments, the nucleic acid construct, and more particularly the expression cassette comprises an intron sequence. This intron sequence can be selected from the group comprising a rice actin intron, a maize hsp70 intron, a maize small subunit RUBISCO intron, a maize ubiquitin intron, a maizeAdh1 intron, a rice phenylalanine ammonia lyase intron, a sucrose synthase intron, a CAT-1 intron, a pKANNIBAL intron, the PIV2 intron and a Super Ubiquitin intron.

In some embodiments, the nucleic acid construct comprises, in particular in the expression cassette, a sequence encoding a selectable marker. This selectable marker is typically used to select transgenic plants containing the DNA construct. When introduced in the nucleic acid construct outside of the expression cassette, the selectable marker can be used to validate the presence of the nucleic acid construct in the transformant (typically the bacterial strain and plants). In preferred embodiment, selectable markers are introduced in the expression cassette and outside of the expression cassette. The selectable marker can be selected from the group consisting of a neomycin phosphotransferase protein, an aminoglycoside 3′-phosphotransferase (Kanamycin resistance protein), a phosphinothricin acetyltransferase protein, a glyphosate resistant 5-enol-pyruvylshikimate-3-phosphate synthase (EPSPS) protein, a hygromycin phosphotransferase protein (hygromycine resistance protein), a dihydropteroate synthase protein, a sulfonylurea insensitive acetolactate synthase protein, an atrazine insensitive Q protein, a nitrilase protein capable of degrading bromoxynil, a dehalogenase protein capable of degrading dalapon, a 2,4-dichloro-phenoxyacetate monoxygenase protein, a methotrexate insensitive dihydrofolate reductase protein, and an aminoethylcysteine insensitive octopine synthase protein.

In some embodiments, the nucleic construct can include, notably in the expression cassette, one or more sequences encoding a peptide linker. Linkers are short peptide sequences composed of flexible residues such as Glycine and serine between adjacent domains to ensure that these domains do not sterically interfere with each other. Linkers are important elements assembling few proteins, ORFs, or peptides together without interfering with the structure of fused units.

Linkers have no negative effects on the activity and stability of the assembled parts in the new structure.

The plant expression cassette described above is typically included in various vectors notably plasmid expression vectors.

Expression vectors contain sequences (see also above) that provide replicative function of the vector and covalently linked sequences in a host cell. For example, bacterial vectors will contain origins of replication that permit replication of the vector in one or more bacterial hosts, as well as in some embodiments, autonomous replication sequences and/or centromeric sequences. Typically, the expression vector further comprises sequences that provide DNA transfer and/or integration functions (e.g., T-DNA border sequences, site specific recombinase recognition sites, and/or integrase recognition sites).

Advantageously, an expression vector of the invention also includes sequences that provide for selective functions (selectable markers such as antibiotic resistance markers, biosynthetic genes), sequences that provide scorable marker functions (e.g., reporter genes), sequences that facilitate in vitro or in vivo manipulations of the sequences (e.g., polylinker sequences, site specific recombination sequences). The scorable marker is typically used to identify transgenic plants containing the DNA construct. The scorable marker can be selected from the group consisting of a beta-glucuronidase protein, a green fluorescent protein, a yellow fluorescent protein, a beta-galactosidase protein, a luciferase protein derived from a luc gene, a luciferase protein derived from a lux gene, a sialidase protein, streptomycin phosphotransferase protein, a nopaline synthase protein, an octopine synthase protein, and a chloramphenicol acetyl transferase protein.

In some embodiments, the synthetic nucleic acid of interest comprises a nucleic acid sequence having at least, 80, 85, 90; 91, 92; 93; 94; 95; 96; 97; 98; 99 and 100% identity with any one of SEQ ID NO:4 to 10.

### Introduction of Expression Vectors in Host Plants

There are various methods of introducing foreign nucleic acids (i.e., nucleic acid of interest) into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276). The principle methods of causing stable integration of exogenous DNA into plant genomic DNA include two main approaches: Agrobacterium-mediated transformation, Rhizobium-mediated transformation, and direct DNA uptake, such as particle-mediated transformation (typically including chemical transfection using cationic polymers or lipid-based nanoparticles such as lipofection including the use of specific transfection reagents such as lipofectamine reagents of jetPRIMER), biolistic methods, DNA transfection, DNA electroporation.
(i) Agrobacterium-mediated gene transfer (see for references Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112); and
(ii) Direct DNA uptake includes (see for references Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68), methods for direct uptake of DNA into protoplasts (Toriyama, K. et al. (1988) Bio/Technology 6:1072-1074); DNA uptake induced by brief electric shock of plant cells (Zhang et al. Plant Cell Rep. (1988) 7:379-384. Fromm et al. Nature (1986) 319:791-793); DNA injection into plant cells or tissues by particle bombardment (Klein et al. Bio/Technology (1988) 6:559-563; McCabe et al. Bio/Technology (1988) 6:923-926; Sanford, Physiol. Plant. (1990) 79:206-209); the use of micropipette systems (Neuhaus et al., Theor. Appl. Genet. (1987) 75:30-36; Neuhaus and Spangenberg, Physiol. Plant. (1990) 79:213-217); glass fibers or silicon carbide whisker transformation of cell cultures, embryos or callus tissue (U.S. Pat. No. 5,464,765) or by) direct incubation of DNA with germinating pollen (DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719).

Agrobacterium spp. (Rhizobiaceae family) are gram-negative bacteria capable of inducing crown gall (A. tumefaciens and A. vitis), hairy root disease (A. rhizogenes), and cane gall (A. rubi) in several plant species. Infectious strains of Agrobacterium, including A. tumefaciens, A. rhizogenes, A. rubi and A. vitis contain plasmids (Ti or Ri plasmids) that are of about 200-kb. These plasmids code for functions associated with i) plasmid replication and maintenance, ii) conjugative transfer, iii) virulence, iv) opine utilization, and v) sensory perception of exogenous signals released by the plant host and neighboring agrobacterial cells at the site of infection.

Genes encoding each of these functions are generally clustered on the plasmid, with the exception of two spatially distinct regions, the virulence or vir region and the transfer-DNA or T-DNA, required for infection of plants, and the tra and trb regions required for conjugative plasmid transfer. The T-DNA of Agrobacterium is approximately 15-20 kbp in length and is integrated into the host plant genome upon its transfer via a process known as recombination. This process utilizes pre-existing gaps in the host plant cell's genome to allow the T-DNA to pair with short sequences in the genome, priming the process of DNA ligation, where the T-DNA is permanently joint to the plant genome. The T-DNA region is flanked at both ends by 24 bp sequences also named boarder sequences. Because the only cis-acting elements required for T-DNA transfer are the left and right border sequences, such T-DNA border sequences can be used to flank any desired sequence of interest (typically an expression cassette as previously described) and introduce it into a host. The T-DNA can thus be deconstructed and used as a natural genetic engineering system by disarming the Ti-plasmid (removing the hormone/opine genes that generate tumors).

Agrobacterium-mediated plant transformation vectors are particularly well-suited to the present invention. In some embodiments, Agrobacterium-mediated plant transformation vectors usable according to the present invention are disarmed Ti-plasmids and typically comprise (i) sequences that permit replication in Agrobacterium but also in other expression system such as bacterial host cell and notably in E Coli as well as (ii) one or more “border” sequences positioned so as to permit integration of the expression cassette into the plant chromosome. These sequences demarcate the DNA segment (T-DNA and notably the synthetic expression cassette previously described) to be transferred into the plant genome. Such Agrobacterium vectors can be adapted for use in either Agrobacterium tumefaciens or Agrobacterium rhizogenes using respectively Tumor (Ti)- or root (Ri)-inducing plasmids.

In some other embodiments, the Ti plasmid can be further deconstructed by introducing a T-DNA region (typically an expression cassette flanked by boarder sequences) into a plasmid to form a transfer DNA (T-DNA) binary system. To facilitate cloning, the T-DNA is thus moved to a shuttle vector that replicates efficiently in various system, notably in bacterial host cells such as Escherichia coli but also contains a low-copy-number origin of replication for maintenance in A. tumefaciens. Systems in which T-DNA and vir genes are located on separate replicons (vectors) are called T-DNA binary systems. T-DNA is located on the binary vector. The vir helper plasmid is considered disarmed if it does not contain oncogenes that could be transferred to a plant.

Thus Agrobacterium-mediated plant transformation vectors usable according to the present invention also include binary vectors. The T-DNA portion of the binary vector, typically a synthetic expression cassette as previously defined, is typically flanked by left and right border sequences and include a transgene (typically an expression cassette as above defined) as well as preferably a plant selectable marker (see above for details). Outside of the T-DNA, the binary vector also typically contains a bacterial selectable marker and an origin of replication (ori) for bacteria. Example binary vectors include pBIN19, pPZP, pCB, pCAMBIA, pGreen, pLSU, and pLX.

The vir helper plasmid contains the vir genes that originated from the Ti plasmid of Agrobacterium. These genes code for a series of proteins that cut the binary vector at the left and right border sequences and facilitate transfer and integration of T-DNA to the plant's cells and genomes, respectively. Several vir helper plasmids have been reported, and common Agrobacterium strains that include vir helper plasmids include for example EHA101, EHA105, AGL-1, LBA4404, and GV2260. The efficiency of transformation can be enhanced by the use of bacterial strains with different degrees of virulence (e.g., GV3101, C58C1, EHA105, LBA4404, and AGL1 are some of the A. tumefaciens strains most commonly used in plant transformation), higher tolerance to recalcitrant tissues or better adaptation to the desired plant species. EHA105, AGL1, and LBA4404 are considered hypervirulent strains, most likely due to increased induction of the vir genes. These strains are recommended for the transformation of recalcitrant or monocot plants, while the milder strains are most often recommended for non-recalcitrant dicotyledonous plants. In some embodiments, the T4SS can be activated or enhanced by the direct addition of acetosyringone (a phenolic of natural or synthetic origin) to the Agrobacterium growth medium (e.g., YEB or LB) and liquid or solid coinoculation medium. Another preconditioning step can be performed by gently incubating (in the dark at 22° C. for 12-16 h) the Agrobacterium cells in Agrobacterium (AB) minimal medium supplemented with acetosyringone (see Basso M F, Arraes F B M, Grossi-de-Sa M, Moreira V J V, Alves-Ferreira M, Grossi-de-Sa M F. Insights Into Genetic and Molecular Elements for Transgenic Crop Development. Front Plant Sci. 2020; 11:509; and notably WO2015099674A1 and Basso M. F., da Cunha B. A. D. B., Ribeiro A. P., Martins P. K., de Souza W. R., de Oliveira N. G., et al. (2017). Improved genetic transformation of sugarcane (Saccharum spp.) embryogenic callus mediated by Agrobacterium tumefaciens. Curr. Protoc. Plant Biol. 2 221-239.).

For detailed literature references on binary vectors usable in Agrobacterium transformation methods, see Lee L Y, Gelvin S B (February 2008). “T-DNA binary vectors and systems”. Plant Physiology. 146 (2): 325-32; Hoekema A, Hirsch P R, Hooykaas P J, Schilperoort R A (May 1983). “A binary plant vector strategy based on separation of vir- and T-region of the Agrobacterium tumefaciens Ti-plasmid”. Nature. 303 (5913): 179-180; Slater A, Scott N, Fowler M (2008). Plant Biotechnology the genetic manipulation of plants. New York: Oxford University Press Inc; Bevan M (November 1984). “Binary Agrobacterium vectors for plant transformation”. Nucleic Acids Research. 12 (22): 8711-21.; Hajdukiewicz P, Svab Z, Maliga P (September 1994). “The small, versatile pPZP family of Agrobacterium binary vectors for plant transformation”. Plant Molecular Biology. 25 (6): 989-94; Xiang C, Han P, Lutziger I, Wang K, Oliver D J (July 1999). “A mini binary vector series for plant transformation”. Plant Molecular Biology. 40 (4): 711-7. doi: 10.1023/a: 1006201910593. PMID 10480394.

Methods of inoculation of the plant tissue vary depending upon the plant species and the Agrobacterium delivery system.

In a first method seeds from the plant to be transformed can be incubated with an Agrobacterium tumefaciens strain containing a T-RNA (such as an expression vector as previously defined). This method, which is also detailed in the Result's section, allows infection of the seeds. The transformed plant expressing the nucleic acid of interest is then regenerated from the seeds. Typically, the target recombinant protein is then expressed in the plant leaves and roots.

In a second method, hypocotyls and cotyledons can be dipped in an Agrobacterium tumefaciens solution, wherein the Agrobacterium tumefaciens strain contains a T-DNA (such as an expression vector as previously defined). The Agrobacterium binds to the hypocotyls and cotyledons an infect them. In such method, the leave cells are transformed. Then the transformed leaves can be cut and grown in tissue cultures to produce GMO plants.

A third method of transforming plants using the Agrobacterium system is protoplast transfection. In this method, protoplasts from leaves are produced using specific enzymes that digest and strip the cell wall of the plant cells, detaching the cells and dispersing them. The detached protoplast forms a round circle shape that is free floating from the rest of the leaves. These free-floating cells can be transformed with T-DNA plasmid using a direct DNA uptake method, such as in a non-limitative manner, electroporation or chemical transfection using for example cationic polymer transfection or lipid-based nanoparticles (e.g., liposomes, optionally coated with polyethylene glycol) or including specific transfection reagents such as lipofectamine reagents (thermofischer) or JetPRIME® (Polyplus) (see for example for review Zhao Y, Huang L. Lipid nanoparticles for gene delivery. Adv Genet. 2014; 88:13-36. doi: 10.1016/B978-0-12-800148-6.00002-X). In some embodiments viral methods can also be used (see also below). Protoplasts are then cell cultured to produce dully grown stable genetically modified (GMO) plants.

A fourth method of producing transgenic plant expressing a recombinant target protein is agroinfiltration. This method uses syringe injection of Agrobacterium tumefaciens into the leaves. Upon infection, the bacterium inserts the T-DNA plasmid carrying the synthetic gene into the leaves. Then using cell-cultures of the leave cells, the transformed leaves can be grown into full stable GMO plants.

A supplementary approach is the leaf disc procedure, which can be performed with any tissue explant that provides a good source for initiation of whole plant differentiation. Typically surface-disinfected leaf discs are incubated with an Agrobacterium strain containing a Ti plasmid. The leaf discs are then transferred to a selective plate on which only transformed cell (thus expressing the selectable marker) grow. See, e.g., for details, Horsch et al. in Plant Molecular Biology Manual A5, Kluwer Academic Publishers, Dordrecht (1988) p. 1-9.

Another supplementary approach employs the Agrobacterium delivery system in combination with vacuum infiltration. The Agrobacterium system is especially viable in the creation of transgenic dicotyledonous plants.

Therefore, in some embodiments, the present invention encompasses a method for producing a genetically modified plant, plant cell, protoplast, or plant tissue comprising a step of:
a) introducing a synthetic plant nucleic acid construct that provides for stable expression of a target protein or poly-peptide into a plant, plant cell, protoplast, or plant tissue by implementing the steps of:
a1) preparing a transformant by introducing the synthetic nucleic acid construct into a bacterial strain; and
a2) transforming a plant, plant cell, a protoplast, or a plant tissue using the transformant;
a′) alternatively the synthetic plant nucleic acid construct can also be introduced using any suitable direct DNA uptake method such as for example described herein (such as electroporation, or the use of specific transfection reagents).
wherein:
the plant is selected from the Spinacia, Lactuca, and Brassica genus, preferably the plant is an edible plant from the Brassica genus.
the nucleic acid construct is a single synthetic construct comprising a regulatory sequence active in a plant for the expression of an operably linked DNA, an operably linked polypeptide or protein-coding DNA molecule, and a 3′ untranslated region;

In preferred embodiment, the strain is typically an Agrobacterium strain, notably an A. tumefaciens strain. This bacterial strain typically comprises (i) a Ti plasmid including a TDNA region comprising or consisting of a synthetic expression cassette as previously defined or (ii) a binary vector system, wherein the binary vector comprises a T-DNA/RNA region comprising or consisting of a synthetic expression cassette as previously defined. In some embodiments the TDNA region comprises a nucleic acid sequence having at least, 80, 85, 90; 91, 92; 93; 94; 95; 96; 97; 98; 99 and 100% identity with SEQ ID NO:4 to 8.

There are various methods of direct DNA transfer into plant cells, including physical methods (such as electroporation) of chemical methods.

In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

The biolistic transformation method (particle bombardment or gene gun) allows the direct introduction of any DNA sequence into the plant genome. For this, the nucleic acid of interest (typically a binary vector as above described) is dehydrated and complexed with small (0.6-1 μM in diameter) gold or tungsten particles (microcarriers). Then, the microcarriers are deposited on the membranes, accelerated with helium gas to a high velocity using a PDS-1000/HeTM or similar system, and bombarded against totipotent plant tissue. Inside the cells, if the DNA has not reached the nucleus, it is disassembled and directed to the nucleus, where it will integrate randomly into the nuclear genome. Gold particles are recommended due to their greater uniformity of size and lack of toxicity (inertness) to plant cells.

The agrolistic method uses the advantages of A. tumefaciens in combination with the high efficiency of DNA delivery achieved with biolistics, allowing increased transformation efficiency. In some embodiments, biolistics using microcarrier particles without DNA can be used to cause minor and superficial injuries. Then, the injured tissue can be co-cultivated with the desired A. tumefaciens strain. For example, microprojectile bombardment before co-cultivation with A. tumefaciens can be used to enhance the transformation efficiency.

Chemical transfection typically includes the use of cationic polymers, or of liposome (lipofection). Cationic polymers typically include DEAE-dextran or polyethylenimine (PEI). The negatively charged DNA binds to the polycation and the complex is taken up by the cell via endocytosis. Lipofection (or liposome transfection) is a technique used to inject genetic material into a cell by means of liposomes, which are vesicles that can easily merge with the cell membrane since they are both made of a phospholipid bilayer. [13] Lipofection generally uses a positively charged (cationic) lipid (cationic liposomes or mixtures) to form an aggregate with the negatively charged (anionic) genetic material.

In some embodiments, introduction of foreign nucleic acids into a plant according to the present application can also be achieved in some embodiment using viruses that have been shown to be useful for the transformation of plant hosts include members of the Caulimoviridae or CaMV. Transformation of plants using plant viruses is described in Gluzman, Y. et al., Communications in Molecular Biology: Viral Vectors, Cold Spring Harbor Laboratory, New York, pp. 172-189 (1988), but see also HARPER, G., HULL, R., LOCKHART, B. AND N. OLSZEWSKI. 2002. Viral sequences integrated into plant genomes. Annu. Rev. Phytopathol. 40:119-136d. doi: 10.1016/j.tplants.2006.08.008. Pseudovirus particles for use in expressing foreign DNA in many hosts, including plants are described in WO 87/06261. According to some embodiments of the invention, the virus used for transient transformations is avirulent and thus is incapable of causing severe symptoms such as reduced growth rate, mosaic, ring spots, leaf roll, yellowing, streaking, pox formation, tumor formation and pitting. A suitable avirulent virus may be a naturally occurring avirulent virus or an artificially attenuated virus. Virus attenuation may be effected by using methods well known in the art including, but not limited to, sub-lethal heating, chemical treatment or by directed mutagenesis techniques such as described, for example, by Kurihara and Watanabe (Molecular Plant Pathology 4:259-269, 2003), Gal-on et al. (1992), Atreya et al. (1992) and Huet et al. (1994). Construction of plant RNA viruses for the introduction and expression of non-viral nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; Takamatsu et al. FEBS Letters (1990) 269:73-76; and U.S. Pat. No. 5,316,931.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Techniques for inoculation of viruses to plants may be found in Foster and Taylor, eds. “Plant Virology Protocols: From Virus Isolation to Transgenic Resistance (Methods in Molecular Biology (Humana Pr), Vol 81)”, Humana Press, 1998; Maramorosh and Koprowski, eds. “Methods in Virology” 7 vols, Academic Press, New York 1967-1984; Hill, S. A. “Methods in Plant Virology”, Blackwell, Oxford, 1984; Walkey, D. G. A. “Applied Plant Virology”, Wiley, New York, 1985; and Kado and Agrawa, eds. “Principles and Techniques in Plant Virology”, Van Nostrand-Reinhold, New York.

Obtaining a Transgenic Plant Comprising the DNA Construct that Stably Expresses a Target Protein or Peptide

This step is typically achieved by regenerating the transgenic plant from the plant, plant cell, protoplast, or plant tissue that received the nucleic acid construct

Therefore, the present application encompasses a method for producing a genetically modified plant, plant cell, protoplast, or plant tissue comprising a step of:
a) introducing a synthetic nucleic acid construct that provides for stable expression of a target protein or poly-peptide into a plant, plant cell, protoplast, or plant tissue;
wherein:
the plant is selected from the Spinacia, Lactuca, and Brassica genus, preferably the plant is an edible plant from the Brassica genus.
the nucleic acid construct is a single synthetic construct comprising a regulatory sequence active in a plant for the expression of an operably linked DNA, an operably linked polypeptide or protein-coding DNA molecule, and a 3′ untranslated region; and
b) obtaining a transgenic plant comprising the DNA construct that stably expresses a target protein or peptide by regenerating the transgenic plant from the plant, plant cell, protoplast, or plant tissue that received the nucleic acid construct. In preferred embodiments, the transgenic plant is regenerated from seed or by using micropropagation.

Following stable transformation plant propagation is typically exercised. The most common method of plant propagation is by seed.

Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

### Method for Obtaining a Plant-Produced Recombinant Protein

The present application further relates to a method for obtaining a plant-produced recombinant protein which comprises the steps of:
producing a genetically modified plant, plant cell, protoplast, or plant tissue expressing a recombinant protein of interest, as previously de fined, and
isolating and optionally purifying said plant produced protein from said genetically modified plant, plant cell, protoplast, or plant tissue.

Processing downstream to the production of transgenic plants expressing a recombinant protein of interest can be divided in two phases: primary recovery and purification, as detailed in Wilken L R, Nikolov Z L. Recovery and purification of plant-made recombinant proteins. Biotechnol Adv. 2012; 30 (2): 419-433.

The objective of primary recovery is notably to maximize product titer and yield in extract or cell homogenate.

The primary recovery steps for leaf and/or seed secreted proteins can comprise steps of:
product release from the biomass by homogenization or aqueous extraction; and/or
solid-liquid separation or fractionation (typically for seed secreted proteins).

For media-secreted proteins, the culture media is typically concentrated before a first chromatography (capture) step.

In some embodiments, the protein of interest, preferably the antibody, is expressed with a yield comprised between 3 and 10 mg, preferably between 4 and 10 mg, more preferably between 4 and 7 mg, mg expressed in terms of protein of interest per gram of fresh leaf weight.

Fractionation and product release: Seed fractionation uses established processing methods, such as dry milling, dry fractionation, and wet milling, to reduce total processing volume and solids content, to enrich the recombinant protein.

Efficient homogenization of plant tissue and disruption of plant cell walls are prerequisites for maximum release of the recombinant protein into the extraction buffer. Extraction optimization may require screening and evaluation of the tissue disruption technique, particle size distribution, buffer composition, plant tissue-to-buffer ratio, and subcellular compartment expression according to classical methods of the skilled person in the field.

Typically the recombinant protein to be purified can be protected from degradation during extraction. To minimize proteolysis and phenol oxidation of the protein during primary recovery, extraction buffers often containing a mixture of protein stabilizing agents such as protease inhibitors, metal chelators, and antioxidants are typically used. Buffer additives such as β-2-mercaptoethanol (B-ME), dithiothreitol (DTT), polyvinyl polypyrrolidone (PVPP), ascorbic acid, and sodium metabisulphite can also be used. Plant protease activities in homogenates and clarified extracts can be avoided by controlling pH, extraction temperature, or by adding protease inhibitors. In some embodiments detergent(s) can be added.

Solid/liquid extraction: Centrifugation is typically used by the skilled person for solids removal and/or clarification of plant extracts and homogenates.

To further remove impurities after extraction, further clarification and pre-treatment steps can be added prior to the purification step, such as aqueous two-phase partitioning, adsorption, precipitation, and/or membrane filtration.

Typically, seed extracts can be clarified by using centrifugation or depth filtration to remove protein and phytic acid precipitates. It can be noted that adjustment of the pH of leafextracts and cell homogenates around to pH 5.0 precipitates the most abundant plant protein (rubisco), cell debris, as well as chlorophyll pigments attached to the protein and debris

The purification phase typically involves a capture step which concentrates the recombinant protein and also removes plant impurities that can be detrimental to protein yield, quality, and/or purification efficiency.

Capture chromatography resins have two primary functions, concentration and partial purification, and should be inexpensive, resistant to chemicals needed for resin regeneration, and able to retain capacity and selectivity over multiple cycles. Resin selection is determined by recombinant protein properties such as charge, hydrophobicity, and biospecificity. Typically, an antibody (i.e., IgG or IgG-based products) can be captured using protein A and/orG columns and further purified with at least one additional chromatography step using resins and process sequences developed for cell culture-derived antibodies (see for example a classical protocol in Fishman J B, Berg E A. Protein A and Protein G Purification of Antibodies. Cold Spring Harb Protoc. 2019; 2019 (1): 10.1101/pdb.prot099143). Further purification steps can include a variety of methods such as ion-exchange, IMAC, HIC, or ceramic hydroxyapatite. The skilled person will typically select the purification method based on the by target protein properties.

Various biospecific (affinity) tags are also well-suited for purification of proteins from plant extracts according to the present invention and have been for example reviewed by Chen Q. in “Expression and purification of pharmaceutical proteins in plants”. (Biol Eng2008; 2:291-321).

### Other Objects of the Invention

The scope of the present application also encompasses a nucleic acid construct as defined herein, optionally in the form of a composition. In particular, the scope of the present application covers the synthetic nucleic acid coding for the protein of interest, as well as the synthetic expression cassette wherein said coding nucleic acid is operably linked to various regulatory sequences. The scope of the present application also covers the expression vector, typically the expression plasmids as herein described, such as notably a Ti plasmid wherein the T-DNA region comprises an expression cassette as previously defined, or a binary vector usable in a binary Agrobacterium system, wherein said binary vector comprises a T-DNA region comprising an expression cassette as previously defined.

The present invention also provides a bacterial strain, optionally in the form of a composition, comprising a nucleic acid construct as herein defined. Typically, the bacterial strain is an Agrobacterium strain, notably an A. tumafaciens strain.

The present invention also provides a genetically modified plant, plant cell, protoplast, or plant tissue from the Brassica genus expressing a nucleic acid construct as herein defined. In particular expressing a Ti plasmid or binary vectors as previously defined.

The nucleic acid construct, bacterial strain and plant of the present invention are typically obtained according to the method described herein.

Finally, the present invention encompasses a plant-produced protein or polypeptide obtained in a method described herein, optionally in the form of a composition, in particular in the form of a pharmaceutical composition.

### Pharmaceutical Compositions

A recombinant plant-produced protein of the present invention can be formulated together with a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). In one embodiment, the carrier should be suitable for subcutaneous route or intratumoral injection. Depending on the route of administration, the active compound, i.e., antibody, immunoconjugate, or bispecific molecule, may be coated in a material to protect the compound from the action of acids and other natural conditions that may inactivate the compound.

Sterile phosphate-buffered saline is one example of a pharmaceutically acceptable carrier. Other suitable carriers are well-known to those in the art. (Remington and Gennaro, 1995) Formulations may further include one or more excipients, preservatives, solubilizers, buffering agents, albumin to prevent protein loss on vial surfaces, etc.

The form of the pharmaceutical compositions, the route of administration, the dosage and the regimen naturally depend upon the condition to be treated, the severity of the illness, the age, weight, and sex of the patient, etc.

The pharmaceutical compositions of the disclosure can be formulated for a topical, oral, parenteral, intranasal, intravenous, intramuscular, subcutaneous or intraocular administration and the like.

Preferably, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of the recombinant protein may be dissolved or dispersed in a pharmaceutically acceptable carrier or aqueous medium.

A recombinant plant-produced protein of the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

### Uses of the Invention

The plant-produced protein of the invention is typically aimed to be used in therapy. The therapeutical use will of course depend on the type of target protein. Preferably the plant-produced recombinant protein is an antibody, notably an anti-immune checkpoint molecule (in particular an anti-PD1). More particularly the present invention is biosimilar for Nivolumab. Therefore, such protein, notably recombinant plant-produced antibody is intended to be use in cancer therapy, optionally in combination with any other cancer-therapy. As mentioned in the present application, it must be understood that the present application encompasses not only classical IgG molecules but also any antigen binding fragment, optionally in multispecific form(s) derived thereof.

In other embodiments, where to protein of interest is an antibody, said recombinant antibody can advantageously used as a biomarker and/or in diagnostic methods, in particular as a diagnostic tools. Diagnostic methods include in vivo, ex vivo and in vitro diagnostic methods.

### Results

### Material and Methods:

### Synthetic Gene Design

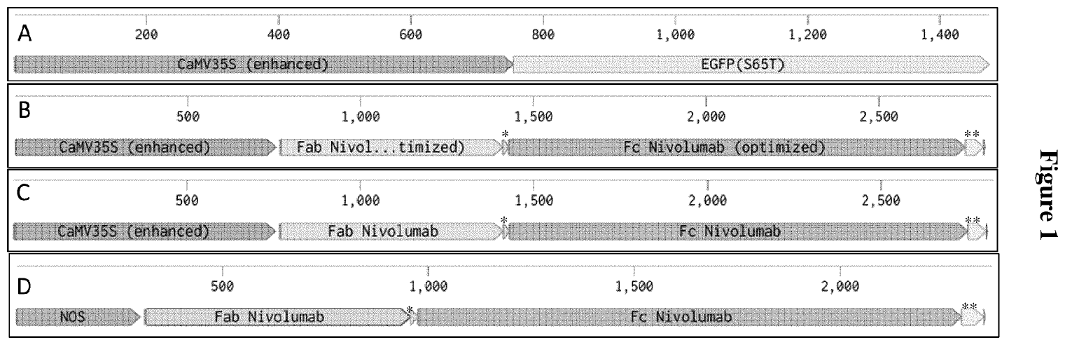

Antibody sequences of Nivolumab were taken from Patent (U.S. Pat. No. 8,779,105B2, doi.org/10.4155/ppa-2017-0015. doi: 10.1080/19420862.2015.1107688) (Protein Seq 1). Using Benchling software the humanized monoclonal antibody sequence was graphically visualized. Start codon was added, two molecular tags of HIS and HA were inserted into the monoclonal antibody (see FIG. 1). Benchling software used to codon optimized the original human DNA sequence for Arabidopsis thaliana (FIG. 1).

### De Novo Synthesis of the Constructs

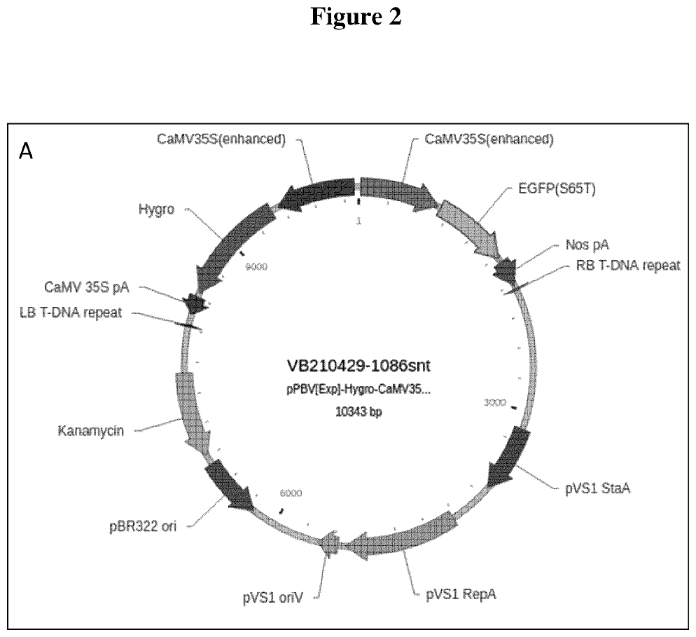

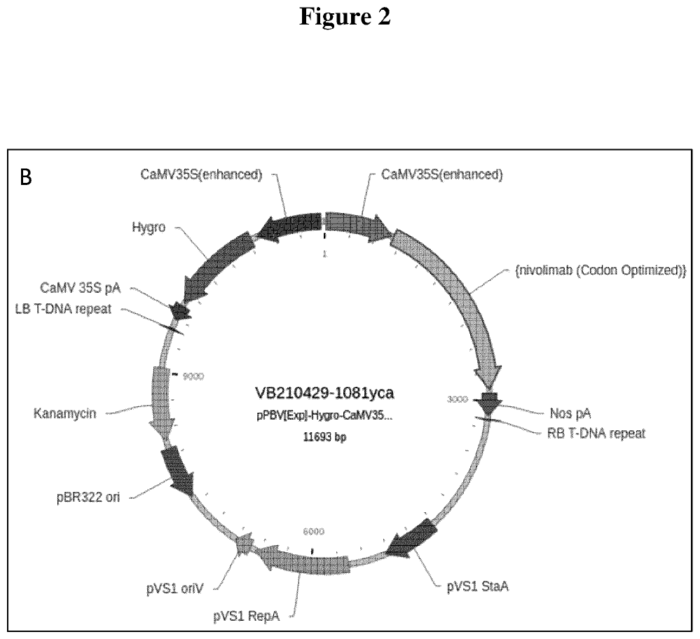

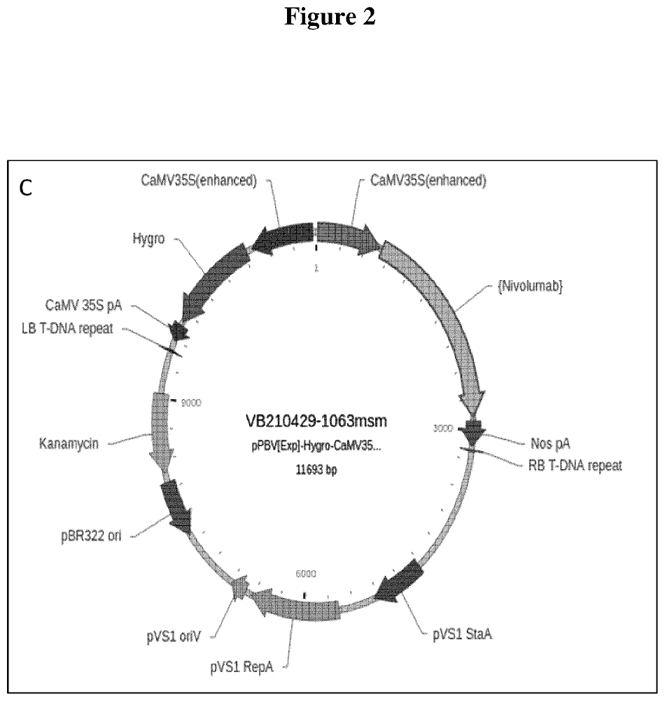

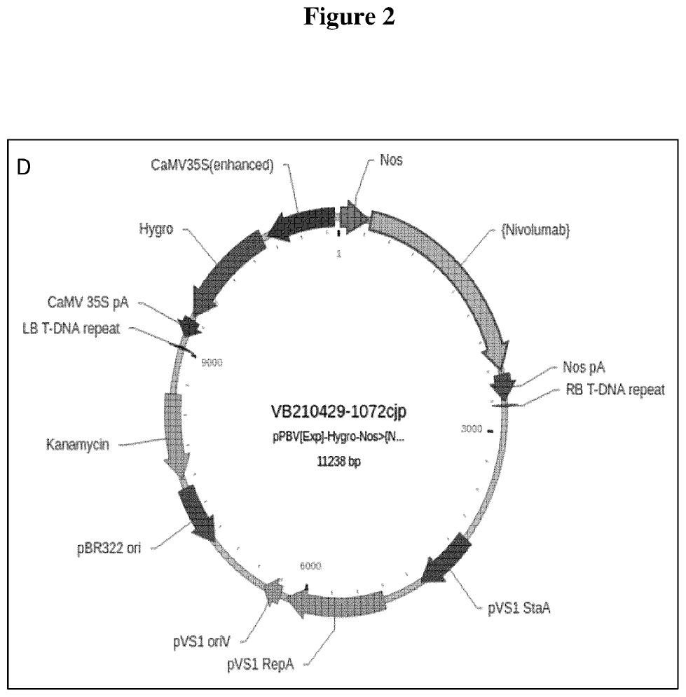

The DNA sequence of the antibody was synthesised by VectorBuilder GmbH (SEQ ID NO4-5). In total four plasmids were synthesised (FIG. 2).

### Cloning in Agrobacterium

The plasmid containing the synthetic gene was cloned into Agrobacterium tumefaciens LBA4404 (Vectorbuilder GmbH) (FIG. 2). All parts of the plasmid have been further described in detail (Table-1).

### Sequencing

The sequencing was performed by Vectorbuilder GmbH. Briefly, the synthetic gene constructs and the plasmids were sequenced using Sanger Sequencing Method. This step verified if the synthesised construct and the plasmids had any point mutation and validated the newly synthesised sequence.

### Restriction Digest

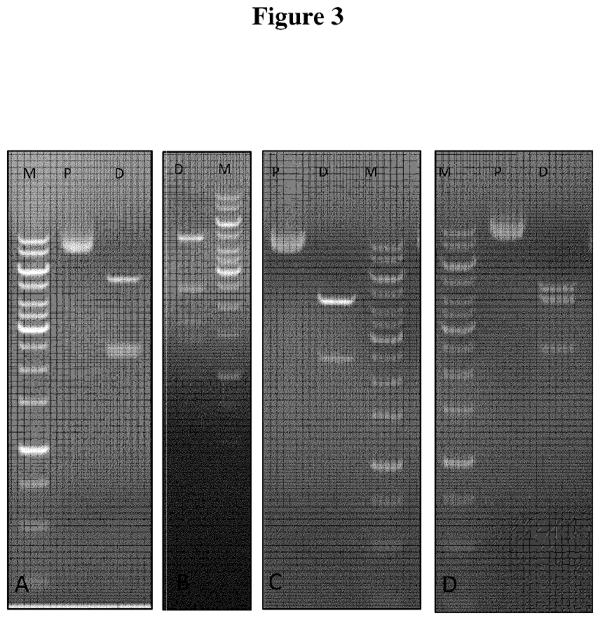

To verify if the synthetic insert was in the proper location, direction, and proper size, a restriction digest was performed. 1 ug of plasmids were incubated with restriction enzymes (New England Biolabs), 1× buffer, 1×BSA, and water to make up a 15 ul reaction. The samples were incubated at the appropriate temperature for 1 hour. Then the solution was electrophoresed on a 1% agarose gel and 1×TAE (ThermoFisher) for 40 minutes at 100V. The gel bands were visualized with Sybr Safe (Thermofischer) (FIG. 3).

### Agrobacterium Cultures

Agrobacterium was cultured on autoclaved Luria-Bertani medium (Sigma) supplemented with 50 ug/mL kanamycin (Sigma). The bacteria were cultured at 25 C for two days in the dark.

### Tissue Lysis:

Leaf punches were flash frozen in liquid nitrogen and crushed in a mortar & pestle. The powdered leaves were put in an eppendorf tube and RIPA buffer (150 mM sodium chloride, 1.0% NP-40 or Triton X-100, 0.5% sodium deoxycholate, 0.1% SDS (sodium dodecyl sulfate), and 50 mM Tris, pH 8.0) was added to lysis the tissue. The samples were incubated for 4 C for 1 hour. The samples were centrifuged at 16 g for 20 minutes. The supernatant was removed and 1×DTT with laemmli was added to the sample. After incubation on ice for 30 minutes, 1× loading buffer (NuPage Thermofisher) was added to the samples. The samples were boiled at 95 C for 10 minutes before dot blot.

### Protoplast: Agroinfiltration

Two days old Agrobacterium cultures were measured on a spectrometer. The cultures were diluted in fresh LB to optical density (OD) of 0.5. 200 ul of bacterial cultures were loaded onto a 1 mL syringe and injected in the leaves of cabbage. The bacteria were allowed to infiltrate the leaf cells for 4-8 days before detecting monoclonal antibodies.

### Dot-Blot:

These were prepared by spotting expressed recombinant proteins on PVDF membranes which were used for western immunodetection. In short: blocking (5% Non-fat milk 1 h); primary antibody (1:1000 HIS, HA 1 h RTP); washing (3×5 min in TBST (Tris buffered saline; 0.1% Tween20); secondary antibody (Donkey anti Mouse 1:1000-1 h RTP); washing (3×5 min in TBST) and detection using WestFemto ECL kit (ThermoFisher). Imaging the blot was performed by GE ImageQuant LAS 500 (FIG. 4).

### Plant Culturing & Growing

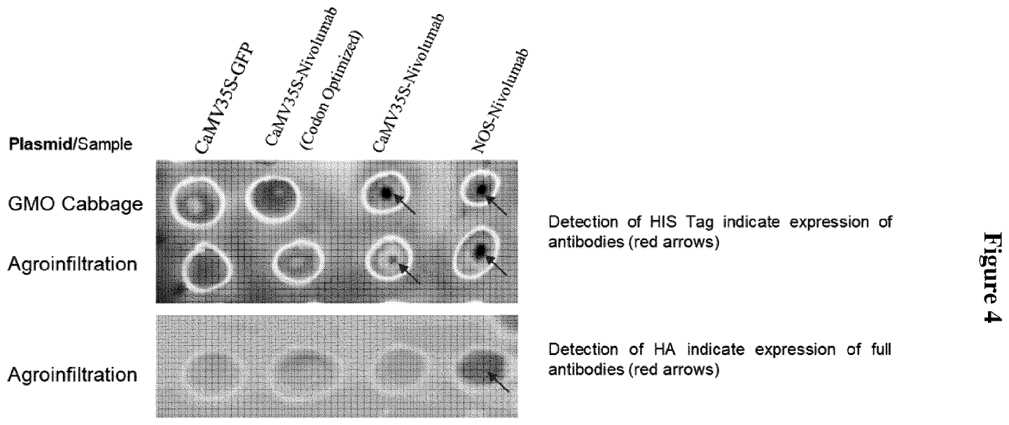

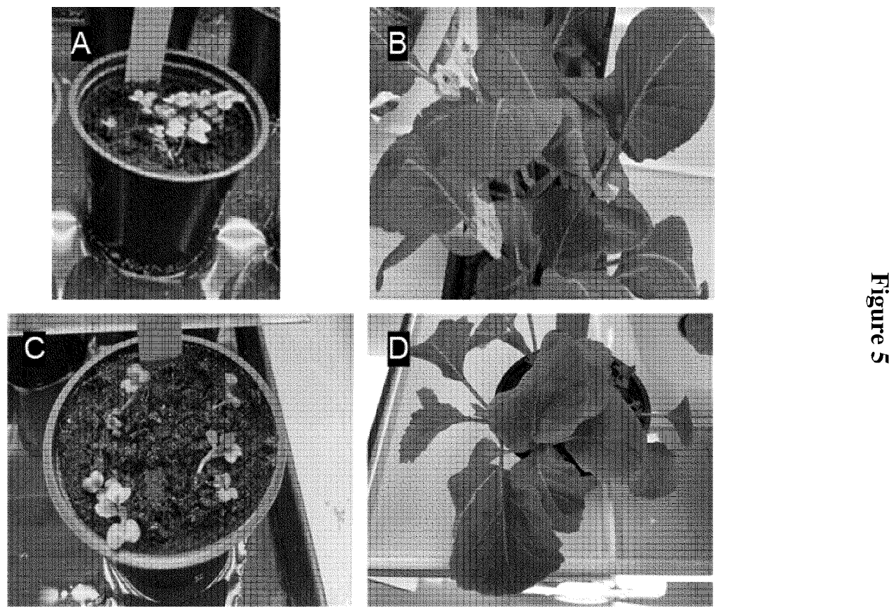

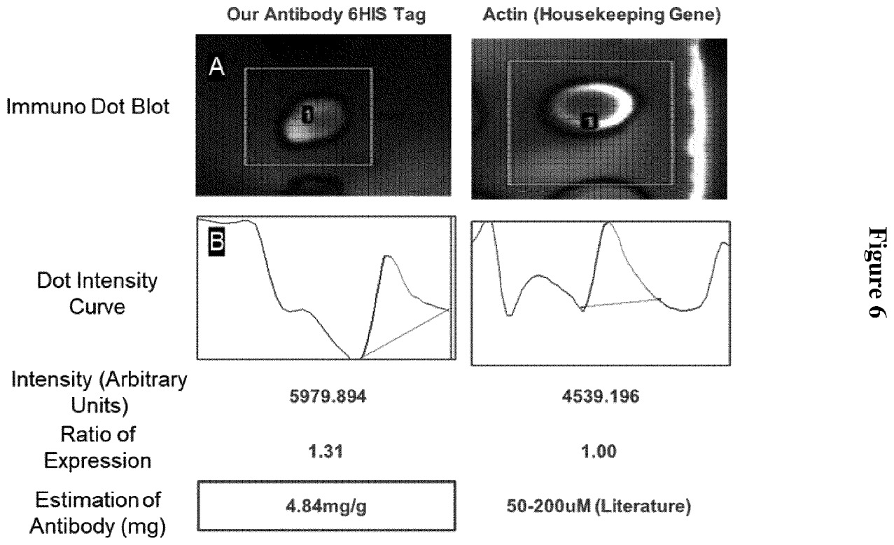

Cabbage plants were potted in small flowerpots with fresh soil. The plants were grown at 25 C 16 h light and 8 hours dark cycles (FIG. 5).

### Signal Quantification

Fresh leaves samples from GMO cabbage plants were weighted on a scale (0.10 g). The leaf sample was grinded and dot blot was performed as above. Using ImageJ, intensity of the chemiluminescence was measured between two antigens. The difference in the intensity between 6×HIS and actin was calculated by the formula:

$$X = 6 XHIS intensity / Actin intensity .$$

### Quantification and Estimation of Antibody Production in GMO Plant:

Total actin concentration in a plant cell is estimated to be around 50-200 uM (see Henty-Ridilla J L, Li J, Blanchoin L, Staiger C J. Actin dynamics in the cortical array of plant cells. Curr Opin Plant Biol. 2013; 16 (6): 678-687. doi: 10.1016/j.pbi.2013.10.012). Based on this estimate value, the estimated amount of 6×HIS present was calculated. Then, the weight of the produced antibody was further estimated from molar concentration using the formulas:

$$m [ g ] = Q [ mol ] x Mw [ kDa ] x 1 {0}^{∧} 3$$

$$c [ M ] x V [ L ] = Q [ mol ]$$

Where: m [g]-weight of protein [gram];
Mw [kDa]-molecular weight of protein [kilo Dalton];
Q [mol]: quantity of protein [mole];
c [M]: molar concentration of protein [Mole]=[mole/litre];
V [L]: dilution volume [litre];

For these calculations the molecular mass of actin used was 42 KDa and 146 KDa for nivolumab. Final weight of the fresh leaf was 0.10 g, while of tissue lysate was 50 μl.

### Additional Results

### Immunoprecipitation (IP)

Dynabeads (Thermofischer) were rotated for 3 minutes. 50 μl of stock Protein A or Protein G dynabeads were removed and placed into a 1.5 ml eppendorf tube. The beads were separated from the solution by placing the tube on a magnet. 100 μl leaf lysate containing the produced antibody thereafter referred to as PiB001 was added to the dynabead for 10 minutes at room temperature with gentle shaking.

Dynabead-PiB001 complex was separated from the leaf tissue lysate by placing it near a magnet. Samples were washed three times with 1×PBS.

### Human Recombinant Antigen

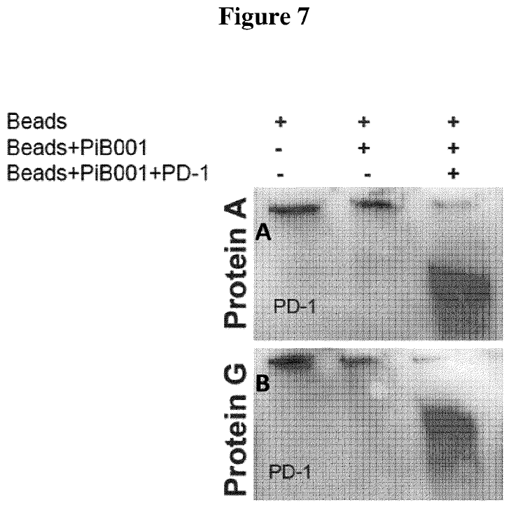

Human recombinant PD-1 protein was purchased from Thermofischer. Briefly, the human recombinant PD-1 was produced in HEK293 cells, corresponding to Accession #NP_005009.2. The C-terminal of the protein has a HIS tag fused to it. The protein was reconstituted following manufacturer guidelines. 1 μl of recombinant PD-1 was dissolved in 100 μl of distilled water and used for Dynabead-PiB001 binding (FIG. 7).

### Human Lung Tumour Lysate

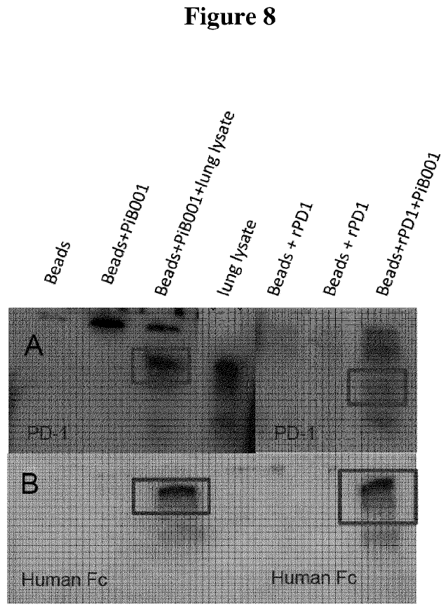

Human lung tumour lysate (Genetex) was diluted lul in 50 μl of 1×PBS and was added to the eppendorf tube containing Dynabeads-PiB001 complex. Crosslinking with PD-1 was performed overnight at 4° C. with rotation (FIG. 8).

By placing the tube on a magnet, the Dynabeads-PiB001-PD-1 complex was separated from the supernatant. The supernatant was transferred to a clean tube. The dynabeads were washed 3 times with 200 μl of Washing Buffer. For each wash the beads were separated from the solution by placing it on a magnet. The beads were suspended in 100 μl of Wash Buffer and transferred to a clean eppendorf tube.

The beads were separated from the solution by placing them on a magnet. 20 μl of Elution Buffer was added to the tube and gently mixed using a pipette.

### Western Blot (WB):

1×NuPage LDS Sample Buffer (4×) (Thermofischer) was added to IP samples. To denature the proteins, the tubes were heated at 95° C. for 10 minutes. Denatured samples were run on 12% SDS Page gel. The gels were run in a tris-Glycine running buffer (Biorad) for 60 minutes at 200V. Membrane transfer was done on a methanol activated PVDF membrane (Thermofischer).

The membrane was blocked (5% Non-fat milk 1 h); primary antibody (1:1000 mouse anti human PD-1 (Thermofischer), 1:1000 mouse anti Human Fc (Genetex) 4 C overnight); washing (3×5 min in TBST (Tris buffered saline; 0.1% Tween20); secondary antibody (Donkey anti Mouse 1:1000-1 h RTP); washing (3×5 min in TBST) and detection using WestFemto ECL kit (ThermoFisher). Imaging the blot was performed by GE ImageQuant LAS 500 (FIG. 7 and FIG. 8).

### Summary of Results:

Immunoprecipitation results demonstrate that the produced antibody PiB001 binds to its antigen, human PD-1. Treating Dynabeads-PiB001 complex with mammalian cell culture (HEK293) produced recombinant PD-1 protein causes PD-1 to bind to PiB001. During the washing steps, unbound recombinant PD-1 is washed away and a new complex of Dynabead-PiB001-PD-1 is formed. Presence of PD-1 is confirmed by Western blot analysis using antibodies against human PD-1 (FIG. 7). Similarly, it was also demonstrated that immunoprecipitation with Protein G was also possible.

Further it was hypothesised that structural difference might exist between recombinant PD-1 and primary tumour PD-1. To demonstrate that PiB001 could also bind to primary human PD-1, immunoprecipitation were performed using lysate of primary human lung tumour. Dynabeads-PiB001 was treated with lysate of primary human lung turnor (Genetex) and unbound proteins were washed away. Western Blot analysis confirmed the immunoprecipitation of PD-1 from lung tumours as well as presence of PiB001 by human Fc antibodies (FIG. 8). Of note, the sample beads+rPD1 leaked into the adjutant lane leading to duplicated lanes (FIG. 8A, right).

TABLE 1

| Name | Type | Description | Application |
|---|---|---|---|
| Nos pA | PolyA_signal | Nopaline synthase | Allows |
| | | polyadenylation | transcription |
| | | signal | termination and |
| | | | polyadenylation |
| | | | of mRNA |
| | | | transcribed by |
| | | | RNA |
| | | | polymerase II. |
| RB T-DNA repeat | Miscellaneous | Right border repeat | Upon |
| | | of T-DNA | recognized by |
| | | | Ti plasmid in |
| | | | Agrobacterium, |
| | | | it transfers the |
| | | | region between |
| | | | T-DNA border |
| | | | repeats to plant |
| | | | cells. |
| pVS1 StaA | ORF | Stability protein | Essential for |
| | | from pVS1 | plasmid |
| | | plasmid | segregational |
| | | | stability in |
| | | | Agrobacterium. |
| pVS1 RepA | ORF | Replication protein | Permits |
| | | from pVS1 | replication of |
| | | plasmid | low-copy |
| | | | plasmid in |
| | | | Agrobacterium. |
| pVS1 oriV | Replication_origin | Origin of | Permits |
| | | replication from | replication of |
| | | pVS1 plasmid | low-copy |
| | | | plasmid in |
| | | | Agrobacterium. |
| pBR322 ori | Replication_origin | pBR322 origin of | Facilitates |
| | | replication | plasmid |
| | | | replication in |
| | | | E. coli; regulates |
| | | | low-copy |
| | | | plasmid number |
| | | | when Rop |
| | | | protein is |
| | | | presence (15- |
| | | | 20 and |
| | | | medium-copy |
| | | | plasmid number |
| | | | when Rop is |
| | | | absent (100- |
| | | | 300). |
| Kanamycin | ORF | Aminoglycoside | Allows E. coli |
| | | 3′- | and |
| | | phosphotransferase | Agrobacterium |
| | | gene. | to be resistant to |
| | | | kanamycin. |
| LB T-DNA repeat | Miscellaneous | Left border repeat | Upon |
| | | of T-DNA | recognized by |
| | | | Ti plasmid in |
| | | | Agrobacterium, |
| | | | it transfers the |
| | | | region between |
| | | | T-DNA border |
| | | | repeats to plant |
| | | | cells. |
| CaMV 35S pA | PolyA_signal | Cauliflower | Allows |
| | | mosaic virus 35S | transcription |
| | | polyadenylation | termination and |
| | | signal | polyadenylation |
| | | | of mRNA |
| | | | transcribed by |
| | | | RNA |
| | | | polymerase II. |
| Hygro | ORF | Hygromycin | Allows plant to |
| | | resistance gene | be resistant to |
| | | | Hygromycin B. |
| CaMV35S(enhanced) | Promoter | A chimeric | Strong |
| | | promoter with the | promoter. |
| | | doubled CaMV | |
| | | 35S enhancer | |
| | | element | |

### Results

The inventors provide genetically modified leafy plants expressing immunotherapy active molecules such as antibodies and recombinant proteins. These antibodies can be used for various purposes, including research and development, clinical, and medical purposes. These plants include, but are not limited to, the genus of Spinacia, Lactuca, Brassica, and all species contained within these mentioned genera.

More particularly, the inventors genetically modified plants (GMOs) to express humanized monoclonal antibody sequences. Using genetic approaches such as synthetic biology and molecular cloning we edited, inserted, modified, and transformed the genomes of plants. Combining plant genetic engineering techniques with synthetic biology, and molecular biology has resulted in novel GMO plants that efficiently express humanized antibodies in all or some of their organs within the root and the shoot system.

De novo produced synthetic DNA was inserted into genetic vectors (vector ID) to express antibodies or recombinant proteins in plants (mentioned above). In addition we genetically modified the ends of synthetic DNA containing the DNA sequence of monoclonal antibody to allow easy detection, enhanced expression, three-dimensional structure folding, and stability of the expressed antibody or recombinant protein.

The DNA sequence of the antibody was modified to make detection of the full-length antibody easy. To do this we inserted two distinct types of genetic/molecular tags into the sequence. One tag was inserted at the end of Fab end, while the other tag was inserted at 3′end of the Fc segment of the antibody (SEQ ID NO:4-5).

The synthetic antibody sequence was driven by two constitutive promoters, either 35CaMVS or NOS, followed by a Kozak consensus sequence. Nopaline synthase polyadenylation signal was inserted at 3′end of the antibody sequence to allow transcription termination and polyadenylation of mRNA by RNA polymerase II.

The genetic vector (VB210429) contained a hygromycin resistance gene under the expression of CaMV35S as a selection marker. This selection marker allows the inventors to screen and identify transfer cells post transfection with the genetic vector.

To make the GMO plants we used the Agrobacterium binary vector system. This system is derived from natural tumor-inducing (Ti) plasmids found in the Agrobacterium. The bacterium transfers a region of the Ti plasmids known as the transfer DNA (T-DNA) into the plant host's nucleus. These Ti plasmids integrate with the host's genome. In our case all tumor forming genes have been removed from the T-DNA and the vector. Only T-DNA border repeats, which flank and direct the host integration remain.

The synthetic antibody sequence was inserted into the T-DNA binary vector, which is bracketed by DNA sequence that will be inserted into the plant host.

This vector along with a second plasmid known as the vir helper plasmid were brought together into Agrobacterium tumefaciens by co-transformation or co-electroporation. These vir helpers encode necessary components for integration of the region flanked by the T-DNA repeat into the genome of the plant cells.

The advantage of such a genetic system is that it allows to insert large DNA inserts into a plant genome that has a high level of expression. In addition, it integrates the antibody sequence permanently into the host plant cells because the T-DNA region is integrated into the host genome.

Stable GMO cabbages expressing monoclonal antibodies were generated by four distinct methods deploying Agrobacterium.

In the first method cabbage seeds were soaked in water. When the seeds absorb water, swell up, and show signs of radicle and Hypocotyls formation, the seeds are treated with Agrobacterium tumefaciens containing the T-DNA carrying the monoclonal antibody. The Agrobacterium infects the growing and the germinating part of the seeds and infecting it with the plasmid expressing the monoclonal antibody. Then the seeds are grown into a plant. The plant leaves and roots infected with the bacterium express the antibody.

In the second method, hypocotyls and cotyledons are dipped in Agrobacterium tumefaciens solution. The Agrobacterium binds to the hypocotyls and cotyledons infecting them. The leaves and roots grown infected cells express monoclonal antibodies.

The third method of producing monoclonal expressing plants is to transfect cabbage protoplast. In this method, protoplasts from young leaves are produced using enzymes. The enzymes digest and strip the cell wall of the plant cells, detaching the cells and dispersing them. The detached protoplast forms a round circle shape that is free floating from the rest of the leaves. These free floating cells are transformed with T-RNA plasmid using electroporation, polyethylene glycol, lipofectamine (thermofischer), and viral methods. This also results in stable GMO plants.

The fourth method of producing cabbage expressing monoclonal Nivolumab is agroinfiltration. This method uses a syringe to inject Agrobacterium tumefaciens into the young leaves. A diluted solution of Agrobacterium tumefaciens is loaded onto a syringe, then using the blunt end of the syringe Agrobacterium tumefaciens is injected, not the leaf. The Agrobacterium tumefaciens is allowed a few days to infect the leaf cells. Upon infection, the bacterium inserts the T-RNA plasmid carrying the synthetic gene into the host cells. Two days post treatment, the leaves express monoclonal antibodies.

Other methods such as ballistics and viral infections can also be used to generate GMO cabbages.

The ballistic method or a ‘Gene Gun’ can also be used for developing the GMO cabbages expressing Nivolumab. The Ti plasmid is attached to nanoparticles and a leaf is bombarded with the nanoparticles carrying the synthetic gene construct. The synthetic construct enters the nucleus and is integrated into the host plant cell.

Finally, in the viral method, synthetic Nivolumab genes are introduced into the host cell using a viral vector. In the method, the Ti plasmid or RNA encoding for Nivolumab gene is packaged into one or multiple plant infecting viruses such as Tobacco mosaic virus, Tomato spotted wilt virus, Tomato yellow leaf curl virus, Cucumber mosaic virus, Potato virus Y, Cauliflower mosaic virus, African cassava mosaic virus, Plum pox virus, Brome mosaic virus and Potato virus X. The cabbage plants are treated with these infecting viruses to introduce the antibody into the plant cell. Once introduced the infected cells express Nivolumab.

Seeds, Calluses, and protoplasts generated from any part of the GMO cabbage or from transfected leaves, cells, or protoplasts expresses Nivolumab. These can be used to propagate, grow, and cultivate GMO plant lines either cultivating GMO seeds or via in vitro cell & tissue cultures. The GMO cabbages can be reproduced asexually.

Any or multiple methods (mentioned above) can be used to generate cabbage plants expressing monoclonal antibodies, especially Nivolumab.

The inventors have thus genetically modified cabbages to express a synthetically constructed plasmid that encodes for Nivolumab monoclonal antibodies. The synthetic gene for this monoclonal antibody was integrated into the host plant, cabbage with the aid of Ti Agrobacterium tumefaciens plasmid. The present results demonstrate that full length monoclonal Nivolumab were produced via seed germination, infecting hypocotyls and cotyledons, and protoplast transformation. In addition, recombinant Nivolumab was also produced by agroinfiltration of the leaf.

These results demonstrate high expression of Nivolumab in the leaves of the cabbage plants. The seeds generated by these plants are also GMO.

Two molecular tags were added to the original monoclonal antibody sequence. A HIS tag was added in 3′ position following the Fab region, while a HA tag was added 3′ to Fc region. These tags serve two critical functions. First these tags are utilized to detect the antibody. Using antibodies specific to these tags, expression level of the monoclonal antibodies can be verified. Secondly, these tags can be utilized to help purify the antibody. These tags can be used in the chelating column to bind to resin and allow for purification of the antibody. Having two tags is adventurous because the samples can be purified sequentially twice for HIS tag and then HA tag. This results in greater purification yields and less wastage.

Typically, the inventors have for example been able to produce according to the present invention 4.84 mg/g of a monoclonal antibody (i.e., nivolumab as herein described). It was observed indeed observed that the 6×HIS tag was overexpressed within a range of 1.15-1.31 times higher in comparison to the housekeeping gene such as actin, within the same sample.

TABLE 2
SEQUENCES

| | |
|---|---|
| SEQ ID NO:1 | QVQLVESGGGVVQPGRSLRLDCKASGITFSNSGMHWVRQAPGKG |
| (Nivolumab | LEWVAVIWYDGSKRYYADSVKGRFTISRDNSKNTLFLQMNSLRA |
| VH sequence) | EDTAVYYCATNDDYWGQGTLVTVSSASTKGPSVFPLAPCSRSTSE |
| | STAALGCLVKDYFPEPVTVSWNSGALTSGVHTFPAVLQSSGLYSL |
| | SSVVTVPSSSLGTKTYTCNVDHKPSNTKVDKRVESKYGPPCPPCP |
| | APEFLGGPSVFLFPPKPKDTLMISRTPEVTCVVVDVSQEDPEVQFN |
| | WYVDGVEVHNAKTKPREEQFNSTYRVVSVLTVLHQDWLNGKEY |
| | KCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLT |
| | VDKSRWQEGNVFSCSVMHEALHNHYTQKSLSLSLGK |
| SEQ ID NO:2 | EIVLTQSPATLSLSPGERATLSCRASQSVSSYLAWYQQKPGQAPRL |
| (Nivolumab | LIYDASNRATGIPARFSGSGSGTDFTLTISSLEPEDFAVYYCQQSSN |
| VL sequence) | WPRTFGQGTKVEIKRTVAAPSVFIFPPSDEQLKSGTASVVCLLNNF |
| | YPREAKVQWKVDNALQSGNSQESVTEQDSKDSTYSLSSTLTLSKA |
| | DYEKHKVYACEVTHQGLSSPVTKSFNRGEC |
| SEQ ID NO:3 | EIVLTQSPATLSLSPGERATLSCRASQSVSSYLAWYQQKPGQAPRL |
| Nivolumab | LIYDASNRATGIPARFSGSGSGTDFTLTISSLEPEDFAVYYCQQSSN |
| construct | WPRTFGQGTKVEIKRTVAAPSVFIFPPSDEQLKSGTASVVCLLNNF |
| sequence | YPREAKVQWKVDNALQSGNSQESVTEQDSKDSTYSLSSTLTLSKA |
| | DYEKHKVYACEVTHQGLSSPVTKSFNRGECHHHHHHQVQLVESG |
| | GGVVQPGRSLRLDCKASGITFSNSGMHWVRQAPGKGLEWVAVI |
| | WYDGSKRYYADSVKGRFTISRDNSKNTLFLQMNSLRAEDTAVYY |
| | CATNDDYWGQGTLVTVSSASTKGPSVFPLAPCSRSTSESTAALGC |
| | LVKDYFPEPVTVSWNSGALTSGVHTFPAVLQSSGLYSLSSVVTVP |
| | SSSLGTKTYTCNVDHKPSNTKVDKRVESKYGPPCPPCPAPEFLGG |
| | PSVFLFPPKPKDTLMISRTPEVTCVVVDVSQEDPEVQFNWYVDGV |
| | EVHNAKTKPREEQFNSTYRVVSVLTVLHQDWLNGKEYKCKVSN |
| | KGLPSSIEKTISKAKGQPREPQVYTLPPSQEEMTKNQVSLTCLVKG |
| | FYPSDIAVEWESNGQPENNYKTTPPVLDSDGSFFLYSRLTVDKSR |
| | WQEGNVFSCSVMHEALHNHYTQKSLSLSLGKYPYDVPDYAYPY |
| | DVPDYA |
| SEQ ID NO:4 | ATGGAAATCGTTCTTACACAGTCACCTGCTACTCTTTCTCTTTC |
| Optimized | ACCAGGTGAGAGAGCTACTCTTTCATGTAGAGCTAGCCAATCT |
| coding | GTTTCTTCTTATTTGGCTTGGTATCAGCAAAAACCTGGTCAAGC |
| sequence for | TCCTAGATTGCTTATCTACGATGCAAGTAACCGGGCTACAGGA |
| Nivolumab | ATTCCTGCTAGATTTTCAGGATCTGGATCCGGTACTGATTTCAC |
| (minusTags) | TCTCACAATTTCCTCTTTGGAACCTGAAGATTTCGCTGTTTATT |
| | ACTGTCAACAATCTAGTAATTGGCCAAGAACTTTTGGACAGGG |
| | AACTAAGGTTGAGATTAAAAGGACTGTTGCTGCTCCTAGTGTG |
| | TTTATCTTTCCTCCATCTGATGAACAATTGAAGTCTGGTACAGC |
| | TTCAGTGGTTTGTCTTCTTAACAATTTTTACCCTAGAGAGGCTA |
| | AAGTTCAGTGGAAAGTGGATAACGCTTTGCAATCTGGTAACTC |
| | TCAAGAATCTGTTACTGAGCAAGATTCCAAAGATAGTACCTAC |
| | TCTCTTAGTTCAACACTTACATTGTCCAAGGCAGATTATGAGAA |
| | ACATAAAGTGTATGCTTGCGAGGTGACTCATCAGGGACTCTCT |
| | TCTCCTGTTACTAAGTCTTTTAACAGAGGAGAGTGTCATCACCA |
| | TCATCATCATCAAGTTCAGTTGGTGGAAAGCGGAGGAGGAGTT |
| | GTTCAGCCTGGTAGGTCTCTCAGACTTGATTGCAAGGCTTCTGG |
| | TATTACTTTTAGTAACTCTGGAATGCATTGGGTTAGACAAGCTC |
| | CTGGAAAAGGTCTTGAATGGGTTGCTGTTATTTGGTATGATGG |
| | ATCTAAGAGATACTACGCTGATTCAGTTAAGGGAAGATTCACA |
| | ATTTCTAGAGATAACTCTAAAAATACTCTTTTCCTTCAAATGAA |
| | TTCATTAAGAGCAGAGGATACTGCAGTGTATTACTGCGCTACC |
| | AATGATGATTATTGGGGTCAGGGAACACTTGTTACTGTTTCTTC |
| | TGCTTCTACTAAAGGACCATCTGTTTTTCCACTGGCTCCTTGTTC |
| | GAGATCTACATCTGAATCAACTGCAGCTCTTGGATGTCTTGTTA |
| | AAGATTATTTCCCAGAGCCTGTTACTGTTTCTTGGAATTCTGGT |
| | GCTCTTACATCTGGTGTTCATACGTTCCCTGCTGTTCTTCAATCT |
| | TCTGGATTGTACTCACTCTCATCTGTTGTTACAGTTCCTTCATCT |
| | AGTCTTGGTACTAAAACATATACATGTAATGTTGATCATAAGC |
| | CTAGTAATACTAAGGTTGATAAAAGAGTTGAGTCCAAGTATGG |
| | TCCACCTTGTCCACCTTGTCCTGCTCCGGAGTTCCTTGGTGGAC |
| | CTTCTGTTTTTCTTTTCCCTCCAAAGCCTAAGGACACTCTTATGA |
| | TTTCGAGAACTCCTGAGGTTACTTGTGTTGTGGTGGATGTGTCA |
| | CAAGAGGATCCTGAAGTTCAGTTTAACTGGTATGTTGATGGAG |
| | TTGAGGTTCATAATGCTAAGACTAAGCCTAGAGAAGAACAATT |
| | CAACTCTACTTACAGAGTTGTTTCAGTGTTAACTGTTCTTCATC |
| | AGGATTGGCTTAATGGTAAGGAGTATAAGTGCAAGGTTTCTAA |
| | CAAGGGACTTCCATCATCAATAGAGAAGACTATTTCTAAGGCT |
| | AAGGGTCAGCCAAGAGAACCTCAGGTTTACACTCTTCCACCTT |
| | CTCAGGAAGAAATGACTAAGAACCAAGTTTCATTAACATGTTT |
| | AGTCAAAGGTTTCTACCCTTCAGATATTGCTGTTGAGTGGGAAT |
| | CTAATGGACAGCCTGAGAACAACTATAAGACTACACCACCTGT |
| | TTTGGATTCTGATGGATCATTTTTCCTTTACTCTAGATTGACAGT |
| | GGATAAATCTAGGTGGCAAGAAGGAAATGTTTTTTCTTGTAGT |
| | GTGATGCATGAGGCGTTGCATAACCATTACACACAAAAAAGTT |
| | TGTCTCTTTCACTTGGTAAGTATCCATACGATGTTCCTGACTAT |
| | GCTTATCCTTATGATGTTCCAGATTATGCTTGA |
| SEQ ID NO:5 | ATGGAAATTGTGCTGACCCAGAGCCCGGCGACCCTGAGCCTGA |
| Non-optimized | GCCCGGGCGAACGCGCGACCCTGAGCTGCCGCGCGAGCCAGA |
| Nivolumab | GCGTGAGCAGCTATCTGGCGTGGTATCAGCAGAAACCGGGCCA |
| (minus Tags) | GGCGCCGCGCCTGCTGATTTATGATGCGAGCAACCGCGCGACC |
| coding | GGCATTCCGGCGCGCTTTAGCGGCAGCGGCAGCGGCACCGATT |
| sequence (nt) | TTACCCTGACCATTAGCAGCCTGGAACCGGAAGATTTTGCGGT |
| | GTATTATTGCCAGCAGAGCAGCAACTGGCCGCGCACCTTTGGC |
| | CAGGGCACCAAAGTGGAAATTAAACGCACCGTGGCGGCGCCG |
| | AGCGTGTTTATTTTTCCGCCGAGCGATGAACAGCTGAAAAGCG |
| | GCACCGCGAGCGTGGTGTGCCTGCTGAACAACTTTTATCCGCG |
| | CGAAGCGAAAGTGCAGTGGAAAGTGGATAACGCGCTGCAGAG |
| | CGGCAACAGCCAGGAAAGCGTGACCGAACAGGATAGCAAAGA |
| | TAGCACCTATAGCCTGAGCAGCACCCTGACCCTGAGCAAAGCG |
| | GATTATGAAAAACATAAAGTGTATGCGTGCGAAGTGACCCATC |
| | AGGGCCTGAGCAGCCCGGTGACCAAAAGCTTTAACCGCGGCGA |
| | ATGCCAGGTGCAGCTGGTGGAAAGCGGCGGCGGCGTGGTGCA |
| | GCCGGGCCGCAGCCTGCGCCTGGATTGCAAAGCGAGCGGCATT |
| | ACCTTTAGCAACAGCGGCATGCATTGGGTGCGCCAGGCGCCGG |
| | GCAAAGGCCTGGAATGGGTGGCGGTGATTTGGTATGATGGCAG |
| | CAAACGCTATTATGCGGATAGCGTGAAAGGCCGCTTTACCATT |
| | AGCCGCGATAACAGCAAAAACACCCTGTTTCTGCAGATGAACA |
| | GCCTGCGCGCGGAAGATACCGCGGTGTATTATTGCGCGACCAA |
| | CGATGATTATTGGGGCCAGGGCACCCTGGTGACCGTGAGCAGC |
| | GCGAGCACCAAAGGCCCGAGCGTGTTTCCGCTGGCGCCGTGCA |
| | GCCGCAGCACCAGCGAAAGCACCGCGGCGCTGGGCTGCCTGGT |
| | GAAAGATTATTTTCCGGAACCGGTGACCGTGAGCTGGAACAGC |
| | GGCGCGCTGACCAGCGGCGTGCATACCTTTCCGGCGGTGCTGC |
| | AGAGCAGCGGCCTGTATAGCCTGAGCAGCGTGGTGACCGTGCC |
| | GAGCAGCAGCCTGGGCACCAAAACCTATACCTGCAACGTGGAT |
| | CATAAACCGAGCAACACCAAAGTGGATAAACGCGTGGAAAGC |
| | AAATATGGCCCGCCGTGCCCGCCGTGCCCGGCGCCGGAATTTC |
| | TGGGCGGCCCGAGCGTGTTTCTGTTTCCGCCGAAACCGAAAGA |
| | TACCCTGATGATTAGCCGCACCCCGGAAGTGACCTGCGTGGTG |
| | GTGGATGTGAGCCAGGAAGATCCGGAAGTGCAGTTTAACTGGT |
| | ATGTGGATGGCGTGGAAGTGCATAACGCGAAAACCAAACCGC |
| | GCGAAGAACAGTTTAACAGCACCTATCGCGTGGTGAGCGTGCT |
| | GACCGTGCTGCATCAGGATTGGCTGAACGGCAAAGAATATAAA |
| | TGCAAAGTGAGCAACAAAGGCCTGCCGAGCAGCATTGAAAAA |
| | ACCATTAGCAAAGCGAAAGGCCAGCCGCGCGAACCGCAGGTG |
| | TATACCCTGCCGCCGAGCCAGGAAGAAATGACCAAAAACCAG |
| | GTGAGCCTGACCTGCCTGGTGAAAGGCTTTTATCCGAGCGATA |
| | TTGCGGTGGAATGGGAAAGCAACGGCCAGCCGGAAAACAACT |
| | ATAAAACCACCCCGCCGGTGCTGGATAGCGATGGCAGCTTTTT |
| | TCTGTATAGCCGCCTGACCGTGGATAAAAGCCGCTGGCAGGAA |
| | GGCAACGTGTTTAGCTGCAGCGTGATGCATGAAGCGCTGCATA |
| | ACCATTATACCCAGAAAAGCCTGAGCCTGAGCCTGGGCAAA |
| SEQ ID NO:6 | GCCAACATGGTGGAGCACGACACTCTCGTCTACTCCAAGAATA |
| Nucleic acid | TCAAAGATACAGTCTCAGAAGACCAAAGGGCTATTGAGACTTT |
| construct | TCAACAAAGGGTAATATCGGGAAACCTCCTCGGATTCCATTGC |
| CaMV35S- | CCAGCTATCTGTCACTTCATCAAAAGGACAGTAGAAAAGGAAG |
| Nivolumab | GTGGCACCTACAAATGCCATCATTGCGATAAAGGAAAGGCTAT |
| (Optimized for | CGTTCAAGATGCCTCTGCCGACAGTGGTCCCAAAGATGGACCC |
| Arabidopsis | CCACCCACGAGGAGCATCGTGGAAAAAGAAGACGTTCCAACC |
| Thaliana but | ACGTCTTCAAAGCAAGTGGATTGATGTGAACATGGTGGAGCAC |
| works in | GACACTCTCGTCTACTCCAAGAATATCAAAGATACAGTCTCAG |
| cabbage) | AAGACCAAAGGGCTATTGAGACTTTTCAACAAAGGGTAATATC |
| +promoter, | GGGAAACCTCCTCGGATTCCATTGCCCAGCTATCTGTCACTTCA |
| +tags | TCAAAAGGACAGTAGAAAAGGAAGGTGGCACCTACAAATGCC |
| | ATCATTGCGATAAAGGAAAGGCTATCGTTCAAGATGCCTCTGC |
| | CGACAGTGGTCCCAAAGATGGACCCCCACCCACGAGGAGCATC |
| | GTGGAAAAAGAAGACGTTCCAACCACGTCTTCAAAGCAAGTGG |
| | ATTGATGTGATATCTCCACTGACGTAAGGGATGACGCACAATC |
| | CCACTATCCTTCGCAAGACCCTTCCTCTATATAAGGAAGTTCAT |
| | TTCATTTGGAGAGGACACGCTGAATGGAAATCGTTCTTACACA |
| | GTCACCTGCTACTCTTTCTCTTTCACCAGGTGAGAGAGCTACTC |
| | TTTCATGTAGAGCTAGCCAATCTGTTTCTTCTTATTTGGCTTGGT |
| | ATCAGCAAAAACCTGGTCAAGCTCCTAGATTGCTTATCTACGA |
| | TGCAAGTAACCGGGCTACAGGAATTCCTGCTAGATTTTCAGGA |
| | TCTGGATCCGGTACTGATTTCACTCTCACAATTTCCTCTTTGGA |
| | ACCTGAAGATTTCGCTGTTTATTACTGTCAACAATCTAGTAATT |
| | GGCCAAGAACTTTTGGACAGGGAACTAAGGTTGAGATTAAAAG |
| | GACTGTTGCTGCTCCTAGTGTGTTTATCTTTCCTCCATCTGATGA |
| | ACAATTGAAGTCTGGTACAGCTTCAGTGGTTTGTCTTCTTAACA |
| | ATTTTTACCCTAGAGAGGCTAAAGTTCAGTGGAAAGTGGATAA |
| | CGCTTTGCAATCTGGTAACTCTCAAGAATCTGTTACTGAGCAAG |
| | ATTCCAAAGATAGTACCTACTCTCTTAGTTCAACACTTACATTG |
| | TCCAAGGCAGATTATGAGAAACATAAAGTGTATGCTTGCGAGG |
| | TGACTCATCAGGGACTCTCTTCTCCTGTTACTAAGTCTTTTAAC |
| | AGAGGAGAGTGTCATCACCATCATCATCATCAAGTTCAGTTGG |
| | TGGAAAGCGGAGGAGGAGTTGTTCAGCCTGGTAGGTCTCTCAG |
| | ACTTGATTGCAAGGCTTCTGGTATTACTTTTAGTAACTCTGGAA |
| | TGCATTGGGTTAGACAAGCTCCTGGAAAAGGTCTTGAATGGGT |
| | TGCTGTTATTTGGTATGATGGATCTAAGAGATACTACGCTGATT |
| | CAGTTAAGGGAAGATTCACAATTTCTAGAGATAACTCTAAAAA |
| | TACTCTTTTCCTTCAAATGAATTCATTAAGAGCAGAGGATACTG |
| | CAGTGTATTACTGCGCTACCAATGATGATTATTGGGGTCAGGG |
| | AACACTTGTTACTGTTTCTTCTGCTTCTACTAAAGGACCATCTG |
| | TTTTTCCACTGGCTCCTTGTTCGAGATCTACATCTGAATCAACT |
| | GCAGCTCTTGGATGTCTTGTTAAAGATTATTTCCCAGAGCCTGT |
| | TACTGTTTCTTGGAATTCTGGTGCTCTTACATCTGGTGTTCATAC |
| | GTTCCCTGCTGTTCTTCAATCTTCTGGATTGTACTCACTCTCATC |
| | TGTTGTTACAGTTCCTTCATCTAGTCTTGGTACTAAAACATATA |
| | CATGTAATGTTGATCATAAGCCTAGTAATACTAAGGTTGATAA |
| | AAGAGTTGAGTCCAAGTATGGTCCACCTTGTCCACCTTGTCCTG |
| | CTCCGGAGTTCCTTGGTGGACCTTCTGTTTTTCTTTTCCCTCCAA |
| | AGCCTAAGGACACTCTTATGATTTCGAGAACTCCTGAGGTTACT |
| | TGTGTTGTGGTGGATGTGTCACAAGAGGATCCTGAAGTTCAGT |
| | TTAACTGGTATGTTGATGGAGTTGAGGTTCATAATGCTAAGACT |
| | AAGCCTAGAGAAGAACAATTCAACTCTACTTACAGAGTTGTTT |
| | CAGTGTTAACTGTTCTTCATCAGGATTGGCTTAATGGTAAGGAG |
| | TATAAGTGCAAGGTTTCTAACAAGGGACTTCCATCATCAATAG |
| | AGAAGACTATTTCTAAGGCTAAGGGTCAGCCAAGAGAACCTCA |
| | GGTTTACACTCTTCCACCTTCTCAGGAAGAAATGACTAAGAAC |
| | CAAGTTTCATTAACATGTTTAGTCAAAGGTTTCTACCCTTCAGA |
| | TATTGCTGTTGAGTGGGAATCTAATGGACAGCCTGAGAACAAC |
| | TATAAGACTACACCACCTGTTTTGGATTCTGATGGATCATTTTT |
| | CCTTTACTCTAGATTGACAGTGGATAAATCTAGGTGGCAAGAA |
| | GGAAATGTTTTTTCTTGTAGTGTGATGCATGAGGCGTTGCATAA |
| | CCATTACACACAAAAAAGTTTGTCTCTTTCACTTGGTAAGTATC |
| | CATACGATGTTCCTGACTATGCTTATCCTTATGATGTTCCAGAT |
| | TATGCTTGA |
| SEQ ID NO:7 | GCCAACATGGTGGAGCACGACACTCTCGTCTACTCCAAGAATA |
| Nucleic acid | TCAAAGATACAGTCTCAGAAGACCAAAGGGCTATTGAGACTTT |
| construct | TCAACAAAGGGTAATATCGGGAAACCTCCTCGGATTCCATTGC |
| CaMV35S- | CCAGCTATCTGTCACTTCATCAAAAGGACAGTAGAAAAGGAAG |
| Nivolumab | GTGGCACCTACAAATGCCATCATTGCGATAAAGGAAAGGCTAT |
| (original) | CGTTCAAGATGCCTCTGCCGACAGTGGTCCCAAAGATGGACCC |
| (with promoter | CCACCCACGAGGAGCATCGTGGAAAAAGAAGACGTTCCAACC |
| tags) | ACGTCTTCAAAGCAAGTGGATTGATGTGAACATGGTGGAGCAC |
| | GACACTCTCGTCTACTCCAAGAATATCAAAGATACAGTCTCAG |
| | AAGACCAAAGGGCTATTGAGACTTTTCAACAAAGGGTAATATC |
| | GGGAAACCTCCTCGGATTCCATTGCCCAGCTATCTGTCACTTCA |
| | TCAAAAGGACAGTAGAAAAGGAAGGTGGCACCTACAAATGCC |
| | ATCATTGCGATAAAGGAAAGGCTATCGTTCAAGATGCCTCTGC |
| | CGACAGTGGTCCCAAAGATGGACCCCCACCCACGAGGAGCATC |
| | GTGGAAAAAGAAGACGTTCCAACCACGTCTTCAAAGCAAGTGG |
| | ATTGATGTGATATCTCCACTGACGTAAGGGATGACGCACAATC |
| | CCACTATCCTTCGCAAGACCCTTCCTCTATATAAGGAAGTTCAT |
| | TTCATTTGGAGAGGACACGCTGAATGGAAATTGTGCTGACCCA |
| | GAGCCCGGCGACCCTGAGCCTGAGCCCGGGCGAACGCGCGAC |
| | CCTGAGCTGCCGCGCGAGCCAGAGCGTGAGCAGCTATCTGGCG |
| | TGGTATCAGCAGAAACCGGGCCAGGCGCCGCGCCTGCTGATTT |
| | ATGATGCGAGCAACCGCGCGACCGGCATTCCGGCGCGCTTTAG |
| | CGGCAGCGGCAGCGGCACCGATTTTACCCTGACCATTAGCAGC |
| | CTGGAACCGGAAGATTTTGCGGTGTATTATTGCCAGCAGAGCA |
| | GCAACTGGCCGCGCACCTTTGGCCAGGGCACCAAAGTGGAAAT |
| | TAAACGCACCGTGGCGGCGCCGAGCGTGTTTATTTTTCCGCCG |
| | AGCGATGAACAGCTGAAAAGCGGCACCGCGAGCGTGGTGTGC |
| | CTGCTGAACAACTTTTATCCGCGCGAAGCGAAAGTGCAGTGGA |
| | AAGTGGATAACGCGCTGCAGAGCGGCAACAGCCAGGAAAGCG |
| | TGACCGAACAGGATAGCAAAGATAGCACCTATAGCCTGAGCA |
| | GCACCCTGACCCTGAGCAAAGCGGATTATGAAAAACATAAAGT |
| | GTATGCGTGCGAAGTGACCCATCAGGGCCTGAGCAGCCCGGTG |
| | ACCAAAAGCTTTAACCGCGGCGAATGCCACCACCACCACCACC |
| | ACCAGGTGCAGCTGGTGGAAAGCGGCGGCGGCGTGGTGCAGC |
| | CGGGCCGCAGCCTGCGCCTGGATTGCAAAGCGAGCGGCATTAC |
| | CTTTAGCAACAGCGGCATGCATTGGGTGCGCCAGGCGCCGGGC |
| | AAAGGCCTGGAATGGGTGGCGGTGATTTGGTATGATGGCAGCA |
| | AACGCTATTATGCGGATAGCGTGAAAGGCCGCTTTACCATTAG |
| | CCGCGATAACAGCAAAAACACCCTGTTTCTGCAGATGAACAGC |
| | CTGCGCGCGGAAGATACCGCGGTGTATTATTGCGCGACCAACG |
| | ATGATTATTGGGGCCAGGGCACCCTGGTGACCGTGAGCAGCGC |
| | GAGCACCAAAGGCCCGAGCGTGTTTCCGCTGGCGCCGTGCAGC |
| | CGCAGCACCAGCGAAAGCACCGCGGCGCTGGGCTGCCTGGTGA |
| | AAGATTATTTTCCGGAACCGGTGACCGTGAGCTGGAACAGCGG |
| | CGCGCTGACCAGCGGCGTGCATACCTTTCCGGCGGTGCTGCAG |
| | AGCAGCGGCCTGTATAGCCTGAGCAGCGTGGTGACCGTGCCGA |
| | GCAGCAGCCTGGGCACCAAAACCTATACCTGCAACGTGGATCA |
| | TAAACCGAGCAACACCAAAGTGGATAAACGCGTGGAAAGCAA |
| | ATATGGCCCGCCGTGCCCGCCGTGCCCGGCGCCGGAATTTCTG |
| | GGCGGCCCGAGCGTGTTTCTGTTTCCGCCGAAACCGAAAGATA |
| | CCCTGATGATTAGCCGCACCCCGGAAGTGACCTGCGTGGTGGT |
| | GGATGTGAGCCAGGAAGATCCGGAAGTGCAGTTTAACTGGTAT |
| | GTGGATGGCGTGGAAGTGCATAACGCGAAAACCAAACCGCGC |
| | GAAGAACAGTTTAACAGCACCTATCGCGTGGTGAGCGTGCTGA |
| | CCGTGCTGCATCAGGATTGGCTGAACGGCAAAGAATATAAATG |
| | CAAAGTGAGCAACAAAGGCCTGCCGAGCAGCATTGAAAAAAC |
| | CATTAGCAAAGCGAAAGGCCAGCCGCGCGAACCGCAGGTGTA |
| | TACCCTGCCGCCGAGCCAGGAAGAAATGACCAAAAACCAGGT |
| | GAGCCTGACCTGCCTGGTGAAAGGCTTTTATCCGAGCGATATT |
| | GCGGTGGAATGGGAAAGCAACGGCCAGCCGGAAAACAACTAT |
| | AAAACCACCCCGCCGGTGCTGGATAGCGATGGCAGCTTTTTTC |
| | TGTATAGCCGCCTGACCGTGGATAAAAGCCGCTGGCAGGAAGG |
| | CAACGTGTTTAGCTGCAGCGTGATGCATGAAGCGCTGCATAAC |
| | CATTATACCCAGAAAAGCCTGAGCCTGAGCCTGGGCAAATACC |
| | CATACGATGTTCCAGATTACGCTTATCCATATGATGTTCCAGAT |
| | TATGCTTAA |
| SEQ ID NO:8 | GATCATGAGCGGAGAATTAAGGGAGTCACGTTATGACCCCCGC |
| Nucleic acid | CGATGACGCGGGACAAGCCGTTTTACGTTTGGAACTGACAGAA |
| construct (Nos- | CCGCAACGTTGAAGGAGCCACTCAGCCGCGGGTTTCTGGAGTT |
| nivolumab) | TAATGAGCTAAGCACATACGTCAGAAACCATTATTGCGCGTTC |
| with promoter | AAAAGTCGCCTAAGGTCACTATCAGCTAGCAAATATTTCTTGTC |
| and tags | AAAAATGCTCCACTGACGTTCCATAAATTCCCCTCGGTATCCAA |
| | TTAGAGTCTCATATTCACTCTCAATCCAAATAATCTGCAATGGA |
| | AATTGTGCTGACCCAGAGCCCGGCGACCCTGAGCCTGAGCCCG |
| | GGCGAACGCGCGACCCTGAGCTGCCGCGCGAGCCAGAGCGTG |
| | AGCAGCTATCTGGCGTGGTATCAGCAGAAACCGGGCCAGGCGC |
| | CGCGCCTGCTGATTTATGATGCGAGCAACCGCGCGACCGGCAT |
| | TCCGGCGCGCTTTAGCGGCAGCGGCAGCGGCACCGATTTTACC |
| | CTGACCATTAGCAGCCTGGAACCGGAAGATTTTGCGGTGTATT |
| | ATTGCCAGCAGAGCAGCAACTGGCCGCGCACCTTTGGCCAGGG |
| | CACCAAAGTGGAAATTAAACGCACCGTGGCGGCGCCGAGCGT |
| | GTTTATTTTTCCGCCGAGCGATGAACAGCTGAAAAGCGGCACC |
| | GCGAGCGTGGTGTGCCTGCTGAACAACTTTTATCCGCGCGAAG |
| | CGAAAGTGCAGTGGAAAGTGGATAACGCGCTGCAGAGCGGCA |
| | ACAGCCAGGAAAGCGTGACCGAACAGGATAGCAAAGATAGCA |
| | CCTATAGCCTGAGCAGCACCCTGACCCTGAGCAAAGCGGATTA |
| | TGAAAAACATAAAGTGTATGCGTGCGAAGTGACCCATCAGGGC |
| | CTGAGCAGCCCGGTGACCAAAAGCTTTAACCGCGGCGAATGCC |
| | ACCACCACCACCACCACCAGGTGCAGCTGGTGGAAAGCGGCG |
| | GCGGCGTGGTGCAGCCGGGCCGCAGCCTGCGCCTGGATTGCAA |
| | AGCGAGCGGCATTACCTTTAGCAACAGCGGCATGCATTGGGTG |
| | CGCCAGGCGCCGGGCAAAGGCCTGGAATGGGTGGCGGTGATTT |
| | GGTATGATGGCAGCAAACGCTATTATGCGGATAGCGTGAAAGG |
| | CCGCTTTACCATTAGCCGCGATAACAGCAAAAACACCCTGTTT |
| | CTGCAGATGAACAGCCTGCGCGCGGAAGATACCGCGGTGTATT |
| | ATTGCGCGACCAACGATGATTATTGGGGCCAGGGCACCCTGGT |
| | GACCGTGAGCAGCGCGAGCACCAAAGGCCCGAGCGTGTTTCCG |
| | CTGGCGCCGTGCAGCCGCAGCACCAGCGAAAGCACCGCGGCG |
| | CTGGGCTGCCTGGTGAAAGATTATTTTCCGGAACCGGTGACCG |
| | TGAGCTGGAACAGCGGCGCGCTGACCAGCGGCGTGCATACCTT |
| | TCCGGCGGTGCTGCAGAGCAGCGGCCTGTATAGCCTGAGCAGC |
| | GTGGTGACCGTGCCGAGCAGCAGCCTGGGCACCAAAACCTATA |
| | CCTGCAACGTGGATCATAAACCGAGCAACACCAAAGTGGATAA |
| | ACGCGTGGAAAGCAAATATGGCCCGCCGTGCCCGCCGTGCCCG |
| | GCGCCGGAATTTCTGGGCGGCCCGAGCGTGTTTCTGTTTCCGCC |
| | GAAACCGAAAGATACCCTGATGATTAGCCGCACCCCGGAAGTG |
| | ACCTGCGTGGTGGTGGATGTGAGCCAGGAAGATCCGGAAGTGC |
| | AGTTTAACTGGTATGTGGATGGCGTGGAAGTGCATAACGCGAA |
| | AACCAAACCGCGCGAAGAACAGTTTAACAGCACCTATCGCGTG |
| | GTGAGCGTGCTGACCGTGCTGCATCAGGATTGGCTGAACGGCA |
| | AAGAATATAAATGCAAAGTGAGCAACAAAGGCCTGCCGAGCA |
| | GCATTGAAAAAACCATTAGCAAAGCGAAAGGCCAGCCGCGCG |
| | AACCGCAGGTGTATACCCTGCCGCCGAGCCAGGAAGAAATGAC |
| | CAAAAACCAGGTGAGCCTGACCTGCCTGGTGAAAGGCTTTTAT |
| | CCGAGCGATATTGCGGTGGAATGGGAAAGCAACGGCCAGCCG |
| | GAAAACAACTATAAAACCACCCCGCCGGTGCTGGATAGCGATG |
| | GCAGCTTTTTTCTGTATAGCCGCCTGACCGTGGATAAAAGCCGC |
| | TGGCAGGAAGGCAACGTGTTTAGCTGCAGCGTGATGCATGAAG |
| | CGCTGCATAACCATTATACCCAGAAAAGCCTGAGCCTGAGCCT |
| | GGGCAAATACCCATACGATGTTCCAGATTACGCTTATCCATAT |
| | GATGTTCCAGATTATGCTTAA |
| 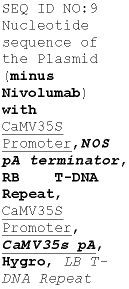 | GCCAACATGGTGGAGCACGACACTCTCGTCTACTCCAAGAATA TCAAAGATACAGTCTCAGAAGACCAAAGGGCTATTGAGACTTT TCAACAAAGGGTAATATCGGGAAACCTCCTCGGATTCCATTGC CCAGCTATCTGTCACTTCATCAAAAGGACAGTAGAAAAGGAAG GTGGCACCTACAAATGCCATCATTGCGATAAAGGAAAGGCTAT CGTTCAAGATGCCTCTGCCGACAGTGGTCCCAAAGATGGACCC CCACCCACGAGGAGCATCGTGGAAAAAGAAGACGTTCCAACC ACGTCTTCAAAGCAAGTGGATTGATGTGAACATGGTGGAGCAC GACACTCTCGTCTACTCCAAGAATATCAAAGATACAGTCTCAG AAGACCAAAGGGCTATTGAGACTTTTCAACAAAGGGTAATATC GGGAAACCTCCTCGGATTCCATTGCCCAGCTATCTGTCACTTCA TCAAAAGGACAGTAGAAAAGGAAGGTGGCACCTACAAATGCC ATCATTGCGATAAAGGAAAGGCTATCGTTCAAGATGCCTCTGC CGACAGTGGTCCCAAAGATGGACCCCCACCCACGAGGAGCATC GTGGAAAAAGAAGACGTTCCAACCACGTCTTCAAAGCAAGTGG ATTGATGTGATATCTCCACTGACGTAAGGGATGACGCACAATC CCACTATCCTTCGCAAGACCCTTCCTCTATATAAGGAAGTTCAT TTCATTTGGAGAGGACACGCTGA[NivolumabSequence]ACC |
| | CAGCTTTCTTGTACAAAGTGGTAGCTCGAATTTCCCCGATCGTTC |
| | AAACATTTGGCAATAAAGTTTCTTAAGATTGAATCCTGTTGCCGG |
| | TCTTGCGATGATTATCATATAATTTCTGTTGAATTACGTTAAGCA |
| | TGTAATAATTAACATGTAATGCATGACGTTATTTATGAGATGGGT |
| | TTTTATGATTAGAGTCCCGCAATTATACATTTAATACGCGATAGA |
| | AAACAAAATATAGCGCGCAAACTAGGATAAATTATCGCGCGCGGT |
| | GTCATCTATGTTACTAGATCGGGAAAGCTTTTAAACTATCAGTGT |
| | 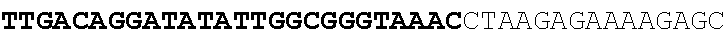 |
| | GTTTATTAGAATAATCGGATATTTAAAAGGGCGTGAAAAGGTT |
| | TATCCGTTCGTCCATTTGTATGTGCATGCCAACCACAGGGTTCC |
| | CCTCGGGATCAAAGTACTTTGATCCAACCCCTCCGCTGCTATAG |
| | TGCAGTCGGCTTCTGACGTTCAGTGCAGCCGTCTTCTGAAAACG |
| | ACATGTCGCACAAGTCCTAAGTTACGCGACAGGCTGCCGCCCT |
| | GCCCTTTTCCTGGCGTTTTCTTGTCGCGTGTTTTAGTCGCATAAA |
| | GTAGAATACTTGCGACTAGAACCGGAGACATTACGCCATGAAC |
| | AAGAGCGCCGCCGCTGGCCTGCTGGGCTATGCCCGCGTCAGCA |
| | CCGACGACCAGGACTTGACCAACCAACGGGCCGAACTGCACGC |
| | GGCCGGCTGCACCAAGCTGTTTTCCGAGAAGATCACCGGCACC |
| | AGGCGCGACCGCCCGGAGCTGGCCAGGATGCTTGACCACCTAC |
| | GCCCTGGCGACGTTGTGACAGTGACCAGGCTAGACCGCCTGGC |
| | CCGCAGCACCCGCGACCTACTGGACATTGCCGAGCGCATCCAG |
| | GAGGCCGGCGCGGGCCTGCGTAGCCTGGCAGAGCCGTGGGCC |
| | GACACCACCACGCCGGCCGGCCGCATGGTGTTGACCGTGTTCG |
| | CCGGCATTGCCGAGTTCGAGCGTTCCCTAATCATCGACCGCAC |
| | CCGGAGCGGGCGCGAGGCCGCCAAGGCCCGAGGCGTGAAGTT |
| | TGGCCCCCGCCCTACCCTCACCCCGGCACAGATCGCGCACGCC |
| | CGCGAGCTGATCGACCAGGAAGGCCGCACCGTGAAAGAGGCG |
| | GCTGCACTGCTTGGCGTGCATCGCTCGACCCTGTACCGCGCACT |
| | TGAGCGCAGCGAGGAAGTGACGCCCACCGAGGCCAGGCGGCG |
| | CGGTGCCTTCCGTGAGGACGCATTGACCGAGGCCGACGCCCTG |
| | GCGGCCGCCGAGAATGAACGCCAAGAGGAACAAGCATGAAAC |
| | CGCACCAGGACGGCCAGGACGAACCGTTTTTCATTACCGAAGA |
| | GATCGAGGCGGAGATGATCGCGGCCGGGTACGTGTTCGAGCCG |
| | CCCGCGCACGTCTCAACCGTGCGGCTGCATGAAATCCTGGCCG |
| | GTTTGTCTGATGCCAAGCTGGCGGCCTGGCCGGCCAGCTTGGC |
| | CGCTGAAGAAACCGAGCGCCGCCGTCTAAAAAGGTGATGTGTA |
| | TTTGAGTAAAACAGCTTGCGTCATGCGGTCGCTGCGTATATGAT |
| | GCGATGAGTAAATAAACAAATACGCAAGGGGAACGCATGAAG |
| | GTTATCGCTGTACTTAACCAGAAAGGCGGGTCAGGCAAGACGA |
| | CCATCGCAACCCATCTAGCCCGCGCCCTGCAACTCGCCGGGGC |
| | CGATGTTCTGTTAGTCGATTCCGATCCCCAGGGCAGTGCCCGCG |
| | ATTGGGCGGCCGTGCGGGAAGATCAACCGCTAACCGTTGTCGG |
| | CATCGACCGCCCGACGATTGACCGCGACGTGAAGGCCATCGGC |
| | CGGCGCGACTTCGTAGTGATCGACGGAGCGCCCCAGGCGGCGG |
| | ACTTGGCTGTGTCCGCGATCAAGGCAGCCGACTTCGTGCTGATT |
| | CCGGTGCAGCCAAGCCCTTACGACATATGGGCCACCGCCGACC |
| | TGGTGGAGCTGGTTAAGCAGCGCATTGAGGTCACGGATGGAAG |
| | GCTACAAGCGGCCTTTGTCGTGTCGCGGGCGATCAAAGGCACG |
| | CGCATCGGCGGTGAGGTTGCCGAGGCGCTGGCCGGGTACGAGC |
| | TGCCCATTCTTGAGTCCCGTATCACGCAGCGCGTGAGCTACCCA |
| | GGCACTGCCGCCGCCGGCACAACCGTTCTTGAATCAGAACCCG |
| | AGGGCGACGCTGCCCGCGAGGTCCAGGCGCTGGCCGCTGAAAT |
| | TAAATCAAAACTCATTTGAGTTAATGAGGTAAAGAGAAAATGA |
| | GCAAAAGCACAAACACGCTAAGTGCCGGCCGTCCGAGCGCAC |
| | GCAGCAGCAAGGCTGCAACGTTGGCCAGCCTGGCAGACACGCC |
| | AGCCATGAAGCGGGTCAACTTTCAGTTGCCGGCGGAGGATCAC |
| | ACCAAGCTGAAGATGTACGCGGTACGCCAAGGCAAGACCATTA |
| | CCGAGCTGCTATCTGAATACATCGCGCAGCTACCAGAGTAAAT |
| | GAGCAAATGAATAAATGAGTAGATGAATTTTAGCGGCTAAAGG |
| | AGGCGGCATGGAAAATCAAGAACAACCAGGCACCGACGCCGT |
| | GGAATGCCCCATGTGTGGAGGAACGGGCGGTTGGCCAGGCGTA |
| | AGCGGCTGGGTTGCCTGCCGGCCCTGCAATGGCACTGGAACCC |
| | CCAAGCCCGAGGAATCGGCGTGAGCGGTCGCAAACCATCCGGC |
| | CCGGTACAAATCGGCGCGGCGCTGGGTGATGACCTGGTGGAGA |
| | AGTTGAAGGCCGCGCAGGCCGCCCAGCGGCAACGCATCGAGG |
| | CAGAAGCACGCCCCGGTGAATCGTGGCAAGCGGCCGCTGATCG |
| | AATCCGCAAAGAATCCCGGCAACCGCCGGCAGCCGGTGCGCCG |
| | TCGATTAGGAAGCCGCCCAAGGGCGACGAGCAACCAGATTTTT |
| | TCGTTCCGATGCTCTATGACGTGGGCACCCGCGATAGTCGCAG |
| | CATCATGGACGTGGCCGTTTTCCGTCTGTCGAAGCGTGACCGA |
| | CGAGCTGGCGAGGTGATCCGCTACGAGCTTCCAGACGGGCACG |
| | TAGAGGTTTCCGCAGGGCCGGCCGGCATGGCCAGTGTGTGGGA |
| | TTACGACCTGGTACTGATGGCGGTTTCCCATCTAACCGAATCCA |
| | TGAACCGATACCGGGAAGGGAAGGGAGACAAGCCCGGCCGCG |
| | TGTTCCGTCCACACGTTGCGGACGTACTCAAGTTCTGCCGGCGA |
| | GCCGATGGCGGAAAGCAGAAAGACGACCTGGTAGAAACCTGC |
| | ATTCGGTTAAACACCACGCACGTTGCCATGCAGCGTACGAAGA |
| | AGGCCAAGAACGGCCGCCTGGTGACGGTATCCGAGGGTGAAG |
| | CCTTGATTAGCCGCTACAAGATCGTAAAGAGCGAAACCGGGCG |
| | GCCGGAGTACATCGAGATCGAGCTAGCTGATTGGATGTACCGC |
| | GAGATCACAGAAGGCAAGAACCCGGACGTGCTGACGGTTCAC |
| | CCCGATTACTTTTTGATCGATCCCGGCATCGGCCGTTTTCTCTA |
| | CCGCCTGGCACGCCGCGCCGCAGGCAAGGCAGAAGCCAGATG |
| | GTTGTTCAAGACGATCTACGAACGCAGTGGCAGCGCCGGAGAG |
| | TTCAAGAAGTTCTGTTTCACCGTGCGCAAGCTGATCGGGTCAA |
| | ATGACCTGCCGGAGTACGATTTGAAGGAGGAGGCGGGGCAGG |
| | CTGGCCCGATCCTAGTCATGCGCTACCGCAACCTGATCGAGGG |
| | CGAAGCATCCGCCGGTTCCTAATGTACGGAGCAGATGCTAGGG |
| | CAAATTGCCCTAGCAGGGGAAAAAGGTCGAAAAGGTCTCTTTC |
| | CTGTGGATAGCACGTACATTGGGAACCCAAAGCCGTACATTGG |
| | GAACCGGAACCCGTACATTGGGAACCCAAAGCCGTACATTGGG |
| | AACCGGTCACACATGTAAGTGACTGATATAAAAGAGAAAAAA |
| | GGCGATTTTTCCGCCTAAAACTCTTTAAAACTTATTAAAACTCT |
| | TAAAACCCGCCTGGCCTGTGCATAACTGTCTGGCCAGCGCACA |
| | GCCGAAGAGCTGCAAAAAGCGCCTACCCTTCGGTCGCTGCGCT |
| | CCCTACGCCCCGCCGCTTCGCGTCGGCCTATCGCGGCCGCTGGC |
| | CGCTCAAAAATGGCTGGCCTACGGCCAGGCAATCTACCAGGGC |
| | GCGGACAAGCCGCGCCGTCGCCACTCGACCGCCGGCGCCCACA |
| | TCAAGGCACCCTGCCTCGCGCGTTTCGGTGATGACGGTGAAAA |
| | CCTCTGACACATGCAGCTCCCGGAGACGGTCACAGCTTGTCTG |
| | TAAGCGGATGCCGGGAGCAGACAAGCCCGTCAGGGCGCGTCA |
| | GCGGGTGTTGGCGGGTGTCGGGGCGCAGCCATGACCCAGTCAC |
| | GTAGCGATAGCGGAGTGTATACTGGCTTAACTATGCGGCATCA |
| | GAGCAGATTGTACTGAGAGTGCACCATATGCGGTGTGAAATAC |
| | CGCACAGATGCGTAAGGAGAAAATACCGCATCAGGCGCTCTTC |
| | CGCTTCCTCGCTCACTGACTCGCTGCGCTCGGTCGTTCGGCTGC |
| | GGCGAGCGGTATCAGCTCACTCAAAGGCGGTAATACGGTTATC |
| | CACAGAATCAGGGGATAACGCAGGAAAGAACATGTGAGCAAA |
| | AGGCCAGCAAAAGGCCAGGAACCGTAAAAAGGCCGCGTTGCT |
| | GGCGTTTTTCCATAGGCTCCGCCCCCCTGACGAGCATCACAAA |
| | AATCGACGCTCAAGTCAGAGGTGGCGAAACCCGACAGGACTAT |
| | AAAGATACCAGGCGTTTCCCCCTGGAAGCTCCCTCGTGCGCTCT |
| | CCTGTTCCGACCCTGCCGCTTACCGGATACCTGTCCGCCTTTCT |
| | CCCTTCGGGAAGCGTGGCGCTTTCTCATAGCTCACGCTGTAGGT |
| | ATCTCAGTTCGGTGTAGGTCGTTCGCTCCAAGCTGGGCTGTGTG |
| | CACGAACCCCCCGTTCAGCCCGACCGCTGCGCCTTATCCGGTA |
| | ACTATCGTCTTGAGTCCAACCCGGTAAGACACGACTTATCGCC |
| | ACTGGCAGCAGCCACTGGTAACAGGATTAGCAGAGCGAGGTAT |
| | GTAGGCGGTGCTACAGAGTTCTTGAAGTGGTGGCCTAACTACG |
| | GCTACACTAGAAGGACAGTATTTGGTATCTGCGCTCTGCTGAA |
| | GCCAGTTACCTTCGGAAAAAGAGTTGGTAGCTCTTGATCCGGC |
| | AAACAAACCACCGCTGGTAGCGGTGGTTTTTTTGTTTGCAAGC |
| | AGCAGATTACGCGCAGAAAAAAAGGATCTCAAGAAGATCCTTT |
| | GATCTTTTCTACGGGGTCTGACGCTCAGTGGAACGAAAACTCA |
| | CGTTAAGGGATTTTGGTCATGCATTCTAGGTACTAAAACAATTC |
| | ATCCAGTAAAATATAATATTTTATTTTCTCCCAATCAGGCTTGA |
| | TCCCCAGTAAGTCAAAAAATAGCTCGACATACTGTTCTTCCCCG |
| | ATATCCTCCCTGATCGACCGGACGCAGAAGGCAATGTCATACC |
| | ACTTGTCCGCCCTGCCGCTTCTCCCAAGATCAATAAAGCCACTT |
| | ACTTTGCCATCTTTCACAAAGATGTTGCTGTCTCCCAGGTCGCC |
| | GTGGGAAAAGACAAGTTCCTCTTCGGGCTTTTCCGTCTTTAAAA |
| | AATCATACAGCTCGCGCGGATCTTTAAATGGAGTGTCTTCTTCC |
| | CAGTTTTCGCAATCCACATCGGCCAGATCGTTATTCAGTAAGTA |
| | ATCCAATTCGGCTAAGCGGCTGTCTAAGCTATTCGTATAGGGA |
| | CAATCCGATATGTCGATGGAGTGAAAGAGCCTGATGCACTCCG |
| | CATACAGCTCGATAATCTTTTCAGGGCTTTGTTCATCTTCATAC |
| | TCTTCCGAGCAAAGGACGCCATCGGCCTCACTCATGAGCAGAT |
| | TGCTCCAGCCATCATGCCGTTCAAAGTGCAGGACCTTTGGAAC |
| | AGGCAGCTTTCCTTCCAGCCATAGCATCATGTCCTTTTCCCGTT |
| | CCACATCATAGGTGGTCCCTTTATACCGGCTGTCCGTCATTTTT |
| | AAATATAGGTTTTCATTTTCTCCCACCAGCTTATATACCTTAGC |
| | AGGAGACATTCCTTCCGTATCTTTTACGCAGCGGTATTTTTCGA |
| | TCAGTTTTTTCAATTCCGGTGATATTCTCATTTTAGCCATTTATT |
| | ATTTCCTTCCTCTTTTCTACAGTATTTAAAGATACCCCAAGAAG |
| | CTAATTATAACAAGACGAACTCCAATTCACTGTTCCTTGCATTC |
| | TAAAACCTTAAATACCAGAAAACAGCTTTTTCAAAGTTGTTTTC |
| | AAAGTTGGCGTATAACATAGTATCGACGGAGCCGATTTTGAAA |
| | CCGCGGTGATCACAGGCAGCAACGCTCTGTCATCGTTACAATC |
| | AACATGCTACCCTCCGCGAGATCATCCGTGTTTCAAACCCGGC |
| | AGCTTAGTTGCCGTTCTTCCGAATAGCATCGGTAACATGAGCA |
| | AAGTCTGCCGCCTTACAACGGCTCTCCCGCTGACGCCGTCCCG |
| | GACTGATGGGCTGCCTGTATCGAGTGGTGATTTTGTGCCGAGCT |
| | 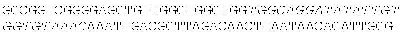 |
| | GACGTTTTTAATGTACTGAATTAACGCCGAATTGAATTCGAGCT |
| | CGGTACCGGGGGATCTGGATTTTAGTACTGGATTTTGGTTTTAG |
| | GAATTAGAAATTTTATTGATAGAAGTATTTTACAAATACAAATAC |
| | ATACTAAGGGTTTCTTATATGCTCAACACATGAGCGAAACCCTAT |
| | AGGAACCCTAATTCCCTTATCTGGGAACTACTCACACATTATTAT |
| | GGAGAAACTCGAGCTTGTCGATCGACAGATCCCGGTCGGCATC |
| | TACTCTATTTCTTTGCCCTCGGACGAGTGCTGGGGCGTCGGTTT |
| | CCACTATCGGCGAGTACTICTACACAGCCATCGGTCCAGACGG |
| | 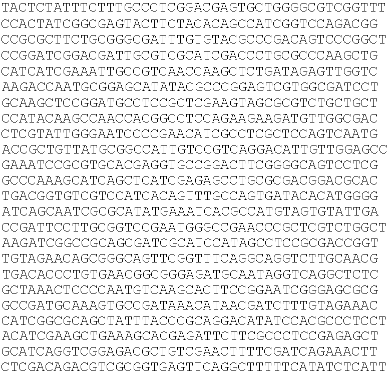 |
| | GCCCCCCGGATCTGCGAAAGCTCGAGAGAGATAGATTTGTAGA |
| | GAGAGACTGGTGATTTCAGCGTGTCCTCTCCAAATGAAATGAA |
| | CTTCCTTATATAGAGGAAGGGTCTTGCGAAGGATAGTGGGATT |
| | GTGCGTCATCCCTTACGTCAGTGGAGATATCACATCAATCCACT |
| | TGCTTTGAAGACGTGGTTGGAACGTCTTCTTTTTCCACGATGCT |
| | CCTCGTGGGTGGGGGTCCATCTTTGGGACCACTGTCGGCAGAG |
| | GCATCTTGAACGATAGCCTTTCCTTTATCGCAATGATGGCATTT |
| | GTAGGTGCCACCTTCCTTTTCTACTGTCCTTTTGATGAAGTGAC |
| | AGATAGCTGGGCAATGGAATCCGAGGAGGTTTCCCGATATTAC |
| | CCTTTGTTGAAAAGTCTCAATAGCCCTTTGGTCTTCTGAGACTG |
| | TATCTTTGATATTCTTGGAGTAGACGAGAGTGTCGTGCTCCACC |
| | ATGTTCACATCAATCCACTTGCTTTGAAGACGTGGTTGGAACGT |
| | CTTCTTTTTCCACGATGCTCCTCGTGGGTGGGGGTCCATCTTTG |
| | GGACCACTGTCGGCAGAGGCATCTTGAACGATAGCCTTTCCTTT |
| | ATCGCAATGATGGCATTTGTAGGTGCCACCTTCCTTTTCTACTG |
| | TCCTTTTGATGAAGTGACAGATAGCTGGGCAATGGAATCCGAG |
| | GAGGTTTCCCGATATTACCCTTTGTTGAAAAGTCTCAATAGCCC |
| | TTTGGTCTTCTGAGACTGTATCTTTGATATTCTTGGAGTAGACG |
| | AGAGTGTCGTGCTCCACCATGTTGGCGTACCCGGGGATCCTCT |
| | AGAGTCGACCTGCAGGCATGCAAGCT |
| 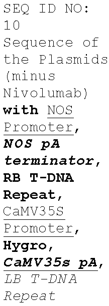 | GATCATGAGCGGAGAATTAAGGGAGTCACGTTATGACCCCCGC CGATGACGCGGGACAAGCCGTTTTACGTTTGGAACTGACAGAA CCGCAACGTTGAAGGAGCCACTCAGCCGCGGGTTTCTGGAGTT TAATGAGCTAAGCACATACGTCAGAAACCATTATTGCGCGTTC AAAAGTCGCCTAAGGTCACTATCAGCTAGCAAATATTTCTTGTC AAAAATGCTCCACTGACGTTCCATAAATTCCCCTCGGTATCCAA TTAGAGTCTCATATTCACTCTCAATCCAAATAATCTGCA[Nivolu mabSequence]ACCCAGCTTTCTTGTACAAAGTGGTAGCTCGAAT TTCCCCGATCGTTCAAACATTTGGCAATAAAGTTTCTTAAGATTG AATCCTGTTGCCGGTCTTGCGATGATTATCATATAATTTCTGTTG AATTACGTTAAGCATGTAATAATTAACATGTAATGCATGACGTTA TTTATGAGATGGGTTTTTATGATTAGAGTCCCGCAATTATACATT TAATACGCGATAGAAAACAAAATATAGCGCGCAAACTAGGATAA ATTATCGCGCGCGGTGTCATCTATGTTACTAGATCGGGAAAGCT 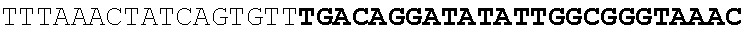 CTAAGAGAAAAGAGCGTTTATTAGAATAATCGGATATTTAAAA GGGCGTGAAAAGGTTTATCCGTTCGTCCATTTGTATGTGCATGC CAACCACAGGGTTCCCCTCGGGATCAAAGTACTTTGATCCAAC CCCTCCGCTGCTATAGTGCAGTCGGCTTCTGACGTTCAGTGCAG |
| | CCGTCTTCTGAAAACGACATGTCGCACAAGTCCTAAGTTACGC |
| | GACAGGCTGCCGCCCTGCCCTTTTCCTGGCGTTTTCTTGTCGCG |
| | TGTTTTAGTCGCATAAAGTAGAATACTTGCGACTAGAACCGGA |
| | GACATTACGCCATGAACAAGAGCGCCGCCGCTGGCCTGCTGGG |
| | CTATGCCCGCGTCAGCACCGACGACCAGGACTTGACCAACCAA |
| | CGGGCCGAACTGCACGCGGCCGGCTGCACCAAGCTGTTTTCCG |
| | AGAAGATCACCGGCACCAGGCGCGACCGCCCGGAGCTGGCCA |
| | GGATGCTTGACCACCTACGCCCTGGCGACGTTGTGACAGTGAC |
| | CAGGCTAGACCGCCTGGCCCGCAGCACCCGCGACCTACTGGAC |
| | ATTGCCGAGCGCATCCAGGAGGCCGGCGCGGGCCTGCGTAGCC |
| | TGGCAGAGCCGTGGGCCGACACCACCACGCCGGCCGGCCGCAT |
| | GGTGTTGACCGTGTTCGCCGGCATTGCCGAGTTCGAGCGTTCCC |
| | TAATCATCGACCGCACCCGGAGCGGGCGCGAGGCCGCCAAGG |
| | CCCGAGGCGTGAAGTTTGGCCCCCGCCCTACCCTCACCCCGGC |
| | ACAGATCGCGCACGCCCGCGAGCTGATCGACCAGGAAGGCCG |
| | CACCGTGAAAGAGGCGGCTGCACTGCTTGGCGTGCATCGCTCG |
| | ACCCTGTACCGCGCACTTGAGCGCAGCGAGGAAGTGACGCCCA |
| | CCGAGGCCAGGCGGCGCGGTGCCTTCCGTGAGGACGCATTGAC |
| | CGAGGCCGACGCCCTGGCGGCCGCCGAGAATGAACGCCAAGA |
| | GGAACAAGCATGAAACCGCACCAGGACGGCCAGGACGAACCG |
| | TTTTTCATTACCGAAGAGATCGAGGCGGAGATGATCGCGGCCG |
| | GGTACGTGTTCGAGCCGCCCGCGCACGTCTCAACCGTGCGGCT |
| | GCATGAAATCCTGGCCGGTTTGTCTGATGCCAAGCTGGCGGCC |
| | TGGCCGGCCAGCTTGGCCGCTGAAGAAACCGAGCGCCGCCGTC |
| | TAAAAAGGTGATGTGTATTTGAGTAAAACAGCTTGCGTCATGC |
| | GGTCGCTGCGTATATGATGCGATGAGTAAATAAACAAATACGC |
| | AAGGGGAACGCATGAAGGTTATCGCTGTACTTAACCAGAAAGG |
| | CGGGTCAGGCAAGACGACCATCGCAACCCATCTAGCCCGCGCC |
| | CTGCAACTCGCCGGGGCCGATGTTCTGTTAGTCGATTCCGATCC |
| | CCAGGGCAGTGCCCGCGATTGGGCGGCCGTGCGGGAAGATCA |
| | ACCGCTAACCGTTGTCGGCATCGACCGCCCGACGATTGACCGC |
| | GACGTGAAGGCCATCGGCCGGCGCGACTTCGTAGTGATCGACG |
| | GAGCGCCCCAGGCGGCGGACTTGGCTGTGTCCGCGATCAAGGC |
| | AGCCGACTTCGTGCTGATTCCGGTGCAGCCAAGCCCTTACGAC |
| | ATATGGGCCACCGCCGACCTGGTGGAGCTGGTTAAGCAGCGCA |
| | TTGAGGTCACGGATGGAAGGCTACAAGCGGCCTTTGTCGTGTC |
| | GCGGGCGATCAAAGGCACGCGCATCGGCGGTGAGGTTGCCGA |
| | GGCGCTGGCCGGGTACGAGCTGCCCATTCTTGAGTCCCGTATC |
| | ACGCAGCGCGTGAGCTACCCAGGCACTGCCGCCGCCGGCACAA |
| | CCGTTCTTGAATCAGAACCCGAGGGCGACGCTGCCCGCGAGGT |
| | CCAGGCGCTGGCCGCTGAAATTAAATCAAAACTCATTTGAGTT |
| | AATGAGGTAAAGAGAAAATGAGCAAAAGCACAAACACGCTAA |
| | GTGCCGGCCGTCCGAGCGCACGCAGCAGCAAGGCTGCAACGTT |
| | GGCCAGCCTGGCAGACACGCCAGCCATGAAGCGGGTCAACTTT |
| | CAGTTGCCGGCGGAGGATCACACCAAGCTGAAGATGTACGCGG |
| | TACGCCAAGGCAAGACCATTACCGAGCTGCTATCTGAATACAT |
| | CGCGCAGCTACCAGAGTAAATGAGCAAATGAATAAATGAGTA |
| | GATGAATTTTAGCGGCTAAAGGAGGCGGCATGGAAAATCAAG |
| | AACAACCAGGCACCGACGCCGTGGAATGCCCCATGTGTGGAGG |
| | AACGGGCGGTTGGCCAGGCGTAAGCGGCTGGGTTGCCTGCCGG |
| | CCCTGCAATGGCACTGGAACCCCCAAGCCCGAGGAATCGGCGT |
| | GAGCGGTCGCAAACCATCCGGCCCGGTACAAATCGGCGCGGCG |
| | CTGGGTGATGACCTGGTGGAGAAGTTGAAGGCCGCGCAGGCCG |
| | CCCAGCGGCAACGCATCGAGGCAGAAGCACGCCCCGGTGAAT |
| | CGTGGCAAGCGGCCGCTGATCGAATCCGCAAAGAATCCCGGCA |
| | ACCGCCGGCAGCCGGTGCGCCGTCGATTAGGAAGCCGCCCAAG |
| | GGCGACGAGCAACCAGATTTTTTCGTTCCGATGCTCTATGACGT |
| | GGGCACCCGCGATAGTCGCAGCATCATGGACGTGGCCGTTTTC |
| | CGTCTGTCGAAGCGTGACCGACGAGCTGGCGAGGTGATCCGCT |
| | ACGAGCTTCCAGACGGGCACGTAGAGGTTTCCGCAGGGCCGGC |
| | CGGCATGGCCAGTGTGTGGGATTACGACCTGGTACTGATGGCG |
| | GTTTCCCATCTAACCGAATCCATGAACCGATACCGGGAAGGGA |
| | AGGGAGACAAGCCCGGCCGCGTGTTCCGTCCACACGTTGCGGA |
| | CGTACTCAAGTTCTGCCGGCGAGCCGATGGCGGAAAGCAGAAA |
| | GACGACCTGGTAGAAACCTGCATTCGGTTAAACACCACGCACG |
| | TTGCCATGCAGCGTACGAAGAAGGCCAAGAACGGCCGCCTGGT |
| | GACGGTATCCGAGGGTGAAGCCTTGATTAGCCGCTACAAGATC |
| | GTAAAGAGCGAAACCGGGCGGCCGGAGTACATCGAGATCGAG |
| | CTAGCTGATTGGATGTACCGCGAGATCACAGAAGGCAAGAACC |
| | CGGACGTGCTGACGGTTCACCCCGATTACTTTTTGATCGATCCC |
| | GGCATCGGCCGTTTTCTCTACCGCCTGGCACGCCGCGCCGCAG |
| | GCAAGGCAGAAGCCAGATGGTTGTTCAAGACGATCTACGAACG |
| | CAGTGGCAGCGCCGGAGAGTTCAAGAAGTTCTGTTTCACCGTG |
| | CGCAAGCTGATCGGGTCAAATGACCTGCCGGAGTACGATTTGA |
| | AGGAGGAGGCGGGGCAGGCTGGCCCGATCCTAGTCATGCGCTA |
| | CCGCAACCTGATCGAGGGCGAAGCATCCGCCGGTTCCTAATGT |
| | ACGGAGCAGATGCTAGGGCAAATTGCCCTAGCAGGGGAAAAA |
| | GGTCGAAAAGGTCTCTTTCCTGTGGATAGCACGTACATTGGGA |
| | ACCCAAAGCCGTACATTGGGAACCGGAACCCGTACATTGGGAA |
| | CCCAAAGCCGTACATTGGGAACCGGTCACACATGTAAGTGACT |
| | GATATAAAAGAGAAAAAAGGCGATTTTTCCGCCTAAAACTCTT |
| | TAAAACTTATTAAAACTCTTAAAACCCGCCTGGCCTGTGCATA |
| | ACTGTCTGGCCAGCGCACAGCCGAAGAGCTGCAAAAAGCGCCT |
| | ACCCTTCGGTCGCTGCGCTCCCTACGCCCCGCCGCTTCGCGTCG |
| | GCCTATCGCGGCCGCTGGCCGCTCAAAAATGGCTGGCCTACGG |
| | CCAGGCAATCTACCAGGGCGCGGACAAGCCGCGCCGTCGCCAC |
| | TCGACCGCCGGCGCCCACATCAAGGCACCCTGCCTCGCGCGTT |
| | TCGGTGATGACGGTGAAAACCTCTGACACATGCAGCTCCCGGA |
| | GACGGTCACAGCTTGTCTGTAAGCGGATGCCGGGAGCAGACAA |
| | GCCCGTCAGGGCGCGTCAGCGGGTGTTGGCGGGTGTCGGGGCG |
| | CAGCCATGACCCAGTCACGTAGCGATAGCGGAGTGTATACTGG |
| | CTTAACTATGCGGCATCAGAGCAGATTGTACTGAGAGTGCACC |
| | ATATGCGGTGTGAAATACCGCACAGATGCGTAAGGAGAAAAT |
| | ACCGCATCAGGCGCTCTTCCGCTTCCTCGCTCACTGACTCGCTG |
| | CGCTCGGTCGTTCGGCTGCGGCGAGCGGTATCAGCTCACTCAA |
| | AGGCGGTAATACGGTTATCCACAGAATCAGGGGATAACGCAG |
| | GAAAGAACATGTGAGCAAAAGGCCAGCAAAAGGCCAGGAACC |
| | GTAAAAAGGCCGCGTTGCTGGCGTTTTTCCATAGGCTCCGCCCC |
| | CCTGACGAGCATCACAAAAATCGACGCTCAAGTCAGAGGTGGC |
| | GAAACCCGACAGGACTATAAAGATACCAGGCGTTTCCCCCTGG |
| | AAGCTCCCTCGTGCGCTCTCCTGTTCCGACCCTGCCGCTTACCG |
| | GATACCTGTCCGCCTTTCTCCCTTCGGGAAGCGTGGCGCTTTCT |
| | CATAGCTCACGCTGTAGGTATCTCAGTTCGGTGTAGGTCGTTCG |
| | CTCCAAGCTGGGCTGTGTGCACGAACCCCCCGTTCAGCCCGAC |
| | CGCTGCGCCTTATCCGGTAACTATCGTCTTGAGTCCAACCCGGT |
| | AAGACACGACTTATCGCCACTGGCAGCAGCCACTGGTAACAGG |
| | ATTAGCAGAGCGAGGTATGTAGGCGGTGCTACAGAGTTCTTGA |
| | AGTGGTGGCCTAACTACGGCTACACTAGAAGGACAGTATTTGG |
| | TATCTGCGCTCTGCTGAAGCCAGTTACCTTCGGAAAAAGAGTT |
| | GGTAGCTCTTGATCCGGCAAACAAACCACCGCTGGTAGCGGTG |
| | GTTTTTTTGTTTGCAAGCAGCAGATTACGCGCAGAAAAAAAGG |
| | ATCTCAAGAAGATCCTTTGATCTTTTCTACGGGGTCTGACGCTC |
| | AGTGGAACGAAAACTCACGTTAAGGGATTTTGGTCATGCATTC |
| | TAGGTACTAAAACAATTCATCCAGTAAAATATAATATTTTATTT |
| | TCTCCCAATCAGGCTTGATCCCCAGTAAGTCAAAAAATAGCTC |
| | GACATACTGTTCTTCCCCGATATCCTCCCTGATCGACCGGACGC |
| | AGAAGGCAATGTCATACCACTTGTCCGCCCTGCCGCTTCTCCCA |
| | AGATCAATAAAGCCACTTACTTTGCCATCTTTCACAAAGATGTT |
| | GCTGTCTCCCAGGTCGCCGTGGGAAAAGACAAGTTCCTCTTCG |
| | GGCTTTTCCGTCTTTAAAAAATCATACAGCTCGCGCGGATCTTT |
| | AAATGGAGTGTCTTCTTCCCAGTTTTCGCAATCCACATCGGCCA |
| | GATCGTTATTCAGTAAGTAATCCAATTCGGCTAAGCGGCTGTCT |
| | AAGCTATTCGTATAGGGACAATCCGATATGTCGATGGAGTGAA |
| | AGAGCCTGATGCACTCCGCATACAGCTCGATAATCTTTTCAGG |
| | GCTTTGTTCATCTTCATACTCTTCCGAGCAAAGGACGCCATCGG |
| | CCTCACTCATGAGCAGATTGCTCCAGCCATCATGCCGTTCAAA |
| | GTGCAGGACCTTTGGAACAGGCAGCTTTCCTTCCAGCCATAGC |
| | ATCATGTCCTTTTCCCGTTCCACATCATAGGTGGTCCCTTTATA |
| | CCGGCTGTCCGTCATTTTTAAATATAGGTTTTCATTTTCTCCCAC |
| | CAGCTTATATACCTTAGCAGGAGACATTCCTTCCGTATCTTTTA |
| | CGCAGCGGTATTTTTCGATCAGTTTTTTCAATTCCGGTGATATT |
| | CTCATTTTAGCCATTTATTATTTCCTTCCTCTTTTCTACAGTATT |
| | TAAAGATACCCCAAGAAGCTAATTATAACAAGACGAACTCCAA |
| | TTCACTGTTCCTTGCATTCTAAAACCTTAAATACCAGAAAACAG |
| | CTTTTTCAAAGTTGTTTTCAAAGTTGGCGTATAACATAGTATCG |
| | ACGGAGCCGATTTTGAAACCGCGGTGATCACAGGCAGCAACGC |
| | TCTGTCATCGTTACAATCAACATGCTACCCTCCGCGAGATCATC |
| | CGTGTTTCAAACCCGGCAGCTTAGTTGCCGTTCTTCCGAATAGC |
| | ATCGGTAACATGAGCAAAGTCTGCCGCCTTACAACGGCTCTCC |
| | CGCTGACGCCGTCCCGGACTGATGGGCTGCCTGTATCGAGTGG |
| | TGATTTTGTGCCGAGCTGCCGGTCGGGGAGCTGTTGGCTGGCT |
| | GGTGGCAGGATATATTGTGGTGTAAACAAATTGACGCTTAGACAA |
| | CTTAATAACACATTGCGGACGTTTTTAATGTACTGAATTAACGC |
| | CGAATTGAATTCGAGCTCGGTACCGGGGGATCTGGATTTTAGTA |
| | CTGGATTTTGGTTTTAGGAATTAGAAATTTTATTGATAGAAGTAT |
| | TTTACAAATACAAATACATACTAAGGGTTTCTTATATGCTCAACA |
| | CATGAGCGAAACCCTATAGGAACCCTAATTCCCTTATCTGGGAA |
| | CTACTCACACATTATTATGGAGAAACTCGAGCTTGTCGATCGAC |
| | 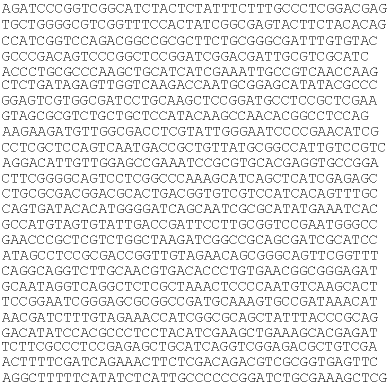 |
| | AGAGAGATAGATTTGTAGAGAGAGACTGGTGATTTCAGCGTGT |
| | CCTCTCCAAATGAAATGAACTTCCTTATATAGAGGAAGGGTCT |
| | TGCGAAGGATAGTGGGATTGTGCGTCATCCCTTACGTCAGTGG |
| | AGATATCACATCAATCCACTTGCTTTGAAGACGTGGTTGGAAC |
| | GTCTTCTTTTTCCACGATGCTCCTCGTGGGTGGGGGTCCATCTT |
| | TGGGACCACTGTCGGCAGAGGCATCTTGAACGATAGCCTTTCC |
| | TTTATCGCAATGATGGCATTTGTAGGTGCCACCTTCCTTTTCTA |
| | CTGTCCTTTTGATGAAGTGACAGATAGCTGGGCAATGGAATCC |
| | GAGGAGGTTTCCCGATATTACCCTTTGTTGAAAAGTCTCAATAG |
| | CCCTTTGGTCTTCTGAGACTGTATCTTTGATATTCTTGGAGTAG |
| | ACGAGAGTGTCGTGCTCCACCATGTTCACATCAATCCACTTGCT |
| | TTGAAGACGTGGTTGGAACGTCTTCTTTTTCCACGATGCTCCTC |
| | GTGGGTGGGGGTCCATCTTTGGGACCACTGTCGGCAGAGGCAT |
| | CTTGAACGATAGCCTTTCCTTTATCGCAATGATGGCATTTGTAG |
| | GTGCCACCTTCCTTTTCTACTGTCCTTTTGATGAAGTGACAGAT |
| | AGCTGGGCAATGGAATCCGAGGAGGTTTCCCGATATTACCCTT |
| | TGTTGAAAAGTCTCAATAGCCCTTTGGTCTTCTGAGACTGTATC |
| | TTTGATATTCTTGGAGTAGACGAGAGTGTCGTGCTCCACCATGT |
| | TGGCGTACCCGGGGATCCTCTAGAGTCGACCTGCAGGCATGCA |
| | AGCT |

1. A method for producing a genetically modified plant, plant cell, protoplast, or plant tissue expressing a recombinant protein of interest, comprising a step of:
a) introducing a plant nucleic acid construct that provides for stable expression of the protein of interest into the plant, plant cell, protoplast, or plant tissue;
wherein:
the plant is selected from the Spinacia, Lactuca, and Brassica genus, and
the nucleic acid construct is a single synthetic construct comprising a regulatory sequence active in a plant for the expression of an operably linked DNA, an operably linked protein-coding DNA molecule, and a 3′ untranslated region.

2. The method according to claim 1 further comprising a step of:
b) obtaining a transgenic plant comprising the DNA construct that stably expresses the protein of interest by regenerating the transgenic plant from the plant, plant cell, protoplast, or plant tissue that received the nucleic acid construct.

3. The method according to claim 1, wherein the nucleic acid construct further comprises one or more of the following sequences: a 5′ untranslated sequence, a signal sequence, an enhancer sequence, a cis-acting element, an intron sequence, a transcriptional Terminator Sequence (TTS), and one or more selectable marker coding sequences.

4. The method according to claim 1, wherein the protein of interest is a multimeric protein.

5. The method according to claim 4, wherein the protein of interest is an antibody and the protein-coding DNA molecule is a single synthetic molecule comprising a nucleic acid sequence coding for an antibody light chain or the antigen-binding fragment thereof and a nucleic acid sequence coding for an antibody heavy chain or the antigen-binding fragment thereof.

6. The method according to claim 4 wherein the protein of interest is a multimeric protein and the multimeric protein-coding nucleic acid synthetic molecule comprises 2 tag sequences located at the 3′ end of each monomer coding sequence.

7. The method according to claim 1 wherein the nucleic acid construct is introduced into the plant or plant cells using
(i) a direct DNA uptake method, or
(ii) Agrobacterium-mediated plant transformation, typically wherein the nucleic acid construct is inserted between the DNA border repeats of an Agrobacterium-mediated plant transformation binary vector (T/DNA binary vector).

8. The method according to claim 1, further comprising the steps of
a1) preparing a transformant by introducing a nucleic acid construct into a bacterial strain; and
a2) transforming the plant, plant cell, protoplast, or plant tissue using the transformant.

9. The method according to claim 8, wherein the strain is an Agrobacterium strain.

10. The method according to claim 9, wherein at step a1) the bacterial strain is obtained using a binary vector system, wherein the bacterial strain is co-transfected with a T/-DNA binary vector and a vic helper plasmid, or wherein a T DNA disarmed A. tumefaciens strain is transfected with a T/DNA binary vector.

11. A method for obtaining a plant produced protein comprising:
producing a genetically modified plant, plant cell, protoplast, or plant tissue expressing a recombinant protein of interest, according to the method of claim 1,
isolating and optionally purifying said plant produced protein from said genetically modified plant, plant cell, protoplast, or plant tissue.

12. (canceled)

13. A bacterial strain comprising a nucleic acid construct-, wherein the nucleic construct is a single synthetic construct comprising a regulatory sequence active in a plant for the expression of an operably linked DNA, an operably linked protein-coding DNA molecule, and a 3′ untranslated region.

14. A genetically modified plant, plant cell, protoplast, or plant tissue from the Brassica genus expressing a nucleic acid construct, wherein the nucleic construct is a single synthetic construct comprising a regulatory sequence active in a plant for the expression of an operably linked DNA, an operably linked protein-coding DNA molecule, and a 3′ untranslated region.

15. A method of treatment comprising administering a plant-produced protein or polypeptide obtained according to a method according to claim 11 to a subject in need thereof.

16. The method for producing a genetically modified plant according to claim 1, wherein the plant is an edible plant from the Brassica genus.

17. The method according to claim 5, wherein the protein of interest is an antibody and the multimeric protein-coding nucleic acid is an antibody-coding DNA synthetic molecule comprising a Tag sequence at the 3′ end of the light chain coding sequence and a Tag sequence at the 3′ end of heavy chain coding sequence.

18. The method according to claim 5, wherein the protein of interest is an antibody and the multimeric protein-coding nucleic acid codes for a protein having at least 90% identity with a sequence of SEQ ID NO: 3 or has at least 60% identity with the nucleic acid sequence of SEQ ID NO: 4 or 5.

19. The method according to claim 7, wherein the nucleic acid construct is inserted between the DNA border repeats of an Agrobacterium-mediated plant transformation binary vector (T/DNA binary vector).

20. A genetically modified plant, plant cell, protoplast, or plant tissue from the Brassica genus transformed with a bacterial strain of claim 13.

21. A genetically modified plant, plant cell, protoplast, or plant tissue from the Brassica genus obtained according to the method of claim 1.


SEQUENCE LISTING (10 sequences)

SEQ ID NO: 1 - protein, 440 aa, synthetic construct
  REGION 1..440: note = VH nivolumab
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, synthetic construct
  REGION 1..214: note = VL Nivolumab
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 678 aa, synthetic construct
  REGION 1..678: note = synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHHQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKYPYDVPDYAYPYDVPDYA

SEQ ID NO: 4 - DNA, 2040 bp, synthetic construct
  misc_feature 1..2040: note = nucleic acid construct
  source 1..2040: mol_type = other DNA
atggaaatcgttcttacacagtcacctgctactctttctctttcaccaggtgagagagctactctttcatgtagagctagccaatctgtttcttcttatttggcttggtatcagcaaaaacctggtcaagctcctagattgcttatctacgatgcaagtaaccgggctacaggaattcctgctagattttcaggatctggatccggtactgatttcactctcacaatttcctctttggaacctgaagatttcgctgtttattactgtcaacaatctagtaattggccaagaacttttggacagggaactaaggttgagattaaaaggactgttgctgctcctagtgtgtttatctttcctccatctgatgaacaattgaagtctggtacagcttcagtggtttgtcttcttaacaatttttaccctagagaggctaaagttcagtggaaagtggataacgctttgcaatctggtaactctcaagaatctgttactgagcaagattccaaagatagtacctactctcttagttcaacacttacattgtccaaggcagattatgagaaacataaagtgtatgcttgcgaggtgactcatcagggactctcttctcctgttactaagtcttttaacagaggagagtgtcatcaccatcatcatcatcaagttcagttggtggaaagcggaggaggagttgttcagcctggtaggtctctcagacttgattgcaaggcttctggtattacttttagtaactctggaatgcattgggttagacaagctcctggaaaaggtcttgaatgggttgctgttatttggtatgatggatctaagagatactacgctgattcagttaagggaagattcacaatttctagagataactctaaaaatactcttttccttcaaatgaattcattaagagcagaggatactgcagtgtattactgcgctaccaatgatgattattggggtcagggaacacttgttactgtttcttctgcttctactaaaggaccatctgtttttccactggctccttgttcgagatctacatctgaatcaactgcagctcttggatgtcttgttaaagattatttcccagagcctgttactgtttcttggaattctggtgctcttacatctggtgttcatacgttccctgctgttcttcaatcttctggattgtactcactctcatctgttgttacagttccttcatctagtcttggtactaaaacatatacatgtaatgttgatcataagcctagtaatactaaggttgataaaagagttgagtccaagtatggtccaccttgtccaccttgtcctgctccggagttccttggtggaccttctgtttttcttttccctccaaagcctaaggacactcttatgatttcgagaactcctgaggttacttgtgttgtggtggatgtgtcacaagaggatcctgaagttcagtttaactggtatgttgatggagttgaggttcataatgctaagactaagcctagagaagaacaattcaactctacttacagagttgtttcagtgttaactgttcttcatcaggattggcttaatggtaaggagtataagtgcaaggtttctaacaagggacttccatcatcaatagagaagactatttctaaggctaagggtcagccaagagaacctcaggtttacactcttccaccttctcaggaagaaatgactaagaaccaagtttcattaacatgtttagtcaaaggtttctacccttcagatattgctgttgagtgggaatctaatggacagcctgagaacaactataagactacaccacctgttttggattctgatggatcatttttcctttactctagattgacagtggataaatctaggtggcaagaaggaaatgttttttcttgtagtgtgatgcatgaggcgttgcataaccattacacacaaaaaagtttgtctctttcacttggtaagtatccatacgatgttcctgactatgcttatccttatgatgttccagattatgcttga

SEQ ID NO: 5 - DNA, 1965 bp, synthetic construct
  misc_feature 1..1965: note = nucleic acid construct
  source 1..1965: mol_type = other DNA
atggaaattgtgctgacccagagcccggcgaccctgagcctgagcccgggcgaacgcgcgaccctgagctgccgcgcgagccagagcgtgagcagctatctggcgtggtatcagcagaaaccgggccaggcgccgcgcctgctgatttatgatgcgagcaaccgcgcgaccggcattccggcgcgctttagcggcagcggcagcggcaccgattttaccctgaccattagcagcctggaaccggaagattttgcggtgtattattgccagcagagcagcaactggccgcgcacctttggccagggcaccaaagtggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgccaggtgcagctggtggaaagcggcggcggcgtggtgcagccgggccgcagcctgcgcctggattgcaaagcgagcggcattacctttagcaacagcggcatgcattgggtgcgccaggcgccgggcaaaggcctggaatgggtggcggtgatttggtatgatggcagcaaacgctattatgcggatagcgtgaaaggccgctttaccattagccgcgataacagcaaaaacaccctgtttctgcagatgaacagcctgcgcgcggaagataccgcggtgtattattgcgcgaccaacgatgattattggggccagggcaccctggtgaccgtgagcagcgcgagcaccaaaggcccgagcgtgtttccgctggcgccgtgcagccgcagcaccagcgaaagcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcaccaaaacctatacctgcaacgtggatcataaaccgagcaacaccaaagtggataaacgcgtggaaagcaaatatggcccgccgtgcccgccgtgcccggcgccggaatttctgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttaacagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaa

SEQ ID NO: 6 - DNA, 2794 bp, synthetic construct
  misc_feature 1..2794: note = nucleic acid construct
  source 1..2794: mol_type = other DNA
gccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaatggaaatcgttcttacacagtcacctgctactctttctctttcaccaggtgagagagctactctttcatgtagagctagccaatctgtttcttcttatttggcttggtatcagcaaaaacctggtcaagctcctagattgcttatctacgatgcaagtaaccgggctacaggaattcctgctagattttcaggatctggatccggtactgatttcactctcacaatttcctctttggaacctgaagatttcgctgtttattactgtcaacaatctagtaattggccaagaacttttggacagggaactaaggttgagattaaaaggactgttgctgctcctagtgtgtttatctttcctccatctgatgaacaattgaagtctggtacagcttcagtggtttgtcttcttaacaatttttaccctagagaggctaaagttcagtggaaagtggataacgctttgcaatctggtaactctcaagaatctgttactgagcaagattccaaagatagtacctactctcttagttcaacacttacattgtccaaggcagattatgagaaacataaagtgtatgcttgcgaggtgactcatcagggactctcttctcctgttactaagtcttttaacagaggagagtgtcatcaccatcatcatcatcaagttcagttggtggaaagcggaggaggagttgttcagcctggtaggtctctcagacttgattgcaaggcttctggtattacttttagtaactctggaatgcattgggttagacaagctcctggaaaaggtcttgaatgggttgctgttatttggtatgatggatctaagagatactacgctgattcagttaagggaagattcacaatttctagagataactctaaaaatactcttttccttcaaatgaattcattaagagcagaggatactgcagtgtattactgcgctaccaatgatgattattggggtcagggaacacttgttactgtttcttctgcttctactaaaggaccatctgtttttccactggctccttgttcgagatctacatctgaatcaactgcagctcttggatgtcttgttaaagattatttcccagagcctgttactgtttcttggaattctggtgctcttacatctggtgttcatacgttccctgctgttcttcaatcttctggattgtactcactctcatctgttgttacagttccttcatctagtcttggtactaaaacatatacatgtaatgttgatcataagcctagtaatactaaggttgataaaagagttgagtccaagtatggtccaccttgtccaccttgtcctgctccggagttccttggtggaccttctgtttttcttttccctccaaagcctaaggacactcttatgatttcgagaactcctgaggttacttgtgttgtggtggatgtgtcacaagaggatcctgaagttcagtttaactggtatgttgatggagttgaggttcataatgctaagactaagcctagagaagaacaattcaactctacttacagagttgtttcagtgttaactgttcttcatcaggattggcttaatggtaaggagtataagtgcaaggtttctaacaagggacttccatcatcaatagagaagactatttctaaggctaagggtcagccaagagaacctcaggtttacactcttccaccttctcaggaagaaatgactaagaaccaagtttcattaacatgtttagtcaaaggtttctacccttcagatattgctgttgagtgggaatctaatggacagcctgagaacaactataagactacaccacctgttttggattctgatggatcatttttcctttactctagattgacagtggataaatctaggtggcaagaaggaaatgttttttcttgtagtgtgatgcatgaggcgttgcataaccattacacacaaaaaagtttgtctctttcacttggtaagtatccatacgatgttcctgactatgcttatccttatgatgttccagattatgcttga

SEQ ID NO: 7 - DNA, 2794 bp, synthetic construct
  misc_feature 1..2794: note = nucleic acid construct
  source 1..2794: mol_type = other DNA
gccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaatggaaattgtgctgacccagagcccggcgaccctgagcctgagcccgggcgaacgcgcgaccctgagctgccgcgcgagccagagcgtgagcagctatctggcgtggtatcagcagaaaccgggccaggcgccgcgcctgctgatttatgatgcgagcaaccgcgcgaccggcattccggcgcgctttagcggcagcggcagcggcaccgattttaccctgaccattagcagcctggaaccggaagattttgcggtgtattattgccagcagagcagcaactggccgcgcacctttggccagggcaccaaagtggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgccaccaccaccaccaccaccaggtgcagctggtggaaagcggcggcggcgtggtgcagccgggccgcagcctgcgcctggattgcaaagcgagcggcattacctttagcaacagcggcatgcattgggtgcgccaggcgccgggcaaaggcctggaatgggtggcggtgatttggtatgatggcagcaaacgctattatgcggatagcgtgaaaggccgctttaccattagccgcgataacagcaaaaacaccctgtttctgcagatgaacagcctgcgcgcggaagataccgcggtgtattattgcgcgaccaacgatgattattggggccagggcaccctggtgaccgtgagcagcgcgagcaccaaaggcccgagcgtgtttccgctggcgccgtgcagccgcagcaccagcgaaagcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcaccaaaacctatacctgcaacgtggatcataaaccgagcaacaccaaagtggataaacgcgtggaaagcaaatatggcccgccgtgcccgccgtgcccggcgccggaatttctgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttaacagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaatacccatacgatgttccagattacgcttatccatatgatgttccagattatgcttaa

SEQ ID NO: 8 - DNA, 2339 bp, synthetic construct
  misc_feature 1..2339: note = nucleic acid construct
  source 1..2339: mol_type = other DNA
gatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatggaaattgtgctgacccagagcccggcgaccctgagcctgagcccgggcgaacgcgcgaccctgagctgccgcgcgagccagagcgtgagcagctatctggcgtggtatcagcagaaaccgggccaggcgccgcgcctgctgatttatgatgcgagcaaccgcgcgaccggcattccggcgcgctttagcggcagcggcagcggcaccgattttaccctgaccattagcagcctggaaccggaagattttgcggtgtattattgccagcagagcagcaactggccgcgcacctttggccagggcaccaaagtggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgccaccaccaccaccaccaccaggtgcagctggtggaaagcggcggcggcgtggtgcagccgggccgcagcctgcgcctggattgcaaagcgagcggcattacctttagcaacagcggcatgcattgggtgcgccaggcgccgggcaaaggcctggaatgggtggcggtgatttggtatgatggcagcaaacgctattatgcggatagcgtgaaaggccgctttaccattagccgcgataacagcaaaaacaccctgtttctgcagatgaacagcctgcgcgcggaagataccgcggtgtattattgcgcgaccaacgatgattattggggccagggcaccctggtgaccgtgagcagcgcgagcaccaaaggcccgagcgtgtttccgctggcgccgtgcagccgcagcaccagcgaaagcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcaccaaaacctatacctgcaacgtggatcataaaccgagcaacaccaaagtggataaacgcgtggaaagcaaatatggcccgccgtgcccgccgtgcccggcgccggaatttctgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttaacagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaatacccatacgatgttccagattacgcttatccatatgatgttccagattatgcttaa

SEQ ID NO: 9 - DNA, 9572 bp, synthetic construct
  misc_feature 1..9572: note = nucleic acid construct
  source 1..9572: mol_type = other DNA
gccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaacccagctttcttgtacaaagtggtagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaaagcttttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcgagctcggtaccgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatcccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgaacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccggatctgcgaaagctcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcgtacccggggatcctctagagtcgacctgcaggcatgcaagct

SEQ ID NO: 10 - DNA, 9117 bp, synthetic construct
  misc_feature 1..9117: note = nucleic acid construct
  source 1..9117: mol_type = other DNA
gatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaacccagctttcttgtacaaagtggtagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaaagcttttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcgagctcggtaccgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatcccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgaacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccggatctgcgaaagctcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcgtacccggggatcctctagagtcgacctgcaggcatgcaagct